(19) United States
(12) Patent Application Publication
(10) Pub. No.: US 2005/0112562 A1
(43) Pub. Date: May 26, 2005

(54) Inhibition of retroviral replication through modulation of the host cell ubiquitylation

(75) Inventors: Donald G. Payan, Hillsborough, CA (US); Yonchu Jenkins, Oakland, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Rigel Pharmaceuticals, Inc.

(21) Appl. No.: 10/985,804

(22) Filed: Nov. 10, 2004

Related U.S. Application Data
(60) Provisional application No. 60/518,863, filed on Nov. 10, 2003.

(51) Int. Cl. C12Q 1/70; A61K 38/17

(52) U.S. Cl. 435/5; 514/2

(57) ABSTRACT

The present invention relates to methods of inhibiting replication of a retrovirus, such as human immunodeficiency virus (HIV), by inhibiting modulation of ubiquitylation of a host cell substrate protein, such as CEM15 of CD4, where ubiquitylation modulation is mediated by a retroviral protein, such as HIV Vif or Vpu. The invention also relates to screening methods for identifying agents that inhibit viral replication by inhibiting retroviral protein-mediated modulation of host cell ubiquitylation.

### FIELD OF THE INVENTION

The present invention relates generally to the field of anti-HIV agents, particularly agents that act by modulating the host cell ubiquitylation pathway in the presence of HIV viral proteins.

### BACKGROUND OF THE INVENTION

Ubiquitin is a highly conserved 76 amino acid protein expressed in all eukaryotic cells. The levels of many intracellular proteins are regulated by a ubiquitin-mediated proteolytic process. This process involves the covalent ligation of ubiquitin to a target protein, resulting in a poly-ubiquitylated target protein which is rapidly detected and degraded by the 26S proteasome.

The ubiquitylation of these target proteins is known to be mediated by the enzymatic activity of three ubiquitin agents. Ubiquitin is first activated in an ATP-dependent manner by a ubiquitin activating agent, for example, an E1. The C-terminus of a ubiquitin forms a high energy thiolester bond with the ubiquitin activating agent.

The ubiquitin is then transferred from the ubiquitin activating agent (E1) to a ubiquitin conjugating agent, e.g., an E2 (also called ubiquitin moiety carrier protein). The ubiquitin is linked to the ubiquitin conjugating agent via a thiolester bond.

The ubiquitin is finally transferred to a target protein (e.g. substrate protein) to form a terminal isopeptide bond with the target protein. The transfer of the ubiquitin to the target protein is mediated by a ubiquitin ligating agent, for example, an E3. Target proteins can be modified by one or more rounds of this process to provide for monomers or oligomers of ubiquitin attached to the target protein. Each ubiquitin of the target protein is covalently ligated to the next ubiquitin through the activity of a ubiquitin ligating agent to form polymers of ubiquitin.

The enzymatic components of the ubiquitylation pathway have received considerable attention (for a review, see, e.g., Wong et al. Drug Discovery Today 8:746-754 (2003); Weissman, Nature Reviews 2:169-178 (2001)). The members of the E1 ubiquitin activating agents and E2 ubiquitin conjugating agents are structurally related and well characterized proteins. There are numerous species of E2 ubiquitin conjugating agents, some of which act in preferred pairs with specific E3 ubiquitin ligating agents to confer specificity for different target proteins. While the nomenclature for the E2 ubiquitin conjugating agents is not standardized across species, investigators in the field have addressed this issue and the skilled artisan can readily identify various E2 ubiquitin conjugating agents, as well as species homologues (See Haas and Siepmann, FASEB J. 11:1257-1268 (1997)).

Ubiquitin agents, such as the ubiquitin activating agents, ubiquitin conjugating agents, and ubiquitin ligating agents, are key determinants of the ubiquitin-mediated proteolytic pathway that results in the degradation of targeted proteins and regulation of cellular processes. Consequently, agents that modulate the activity of such ubiquitin agents can upregulate or downregulate activity of specific host cell proteins.

Recently, there have been reports that human immunodeficiency virus (HIV) proteins modulate degradation of proteins in the infected host cell. For example, the HIV-1 accessory/regulatory protein Vpu modulates CD4 degradation by recruiting the SCF^{βTrCp} ubiquitin E3 ligase to host cell CD4, decreasing availability of CD4 in the cell and enhancing trafficking of HIV envelope protein to the cell surface (Margottin et al. Molec. Cell 1:565-574 (1998)). Although the cellular mechanisms involved have not been elucidated, a recent report indicates that the HIV virion infectivity factor (Vif) induces proteosome-mediated degradation of the host cell protein CEM15, a deaminase which mediates conversion of deoxycytidine residues in retroviral nucleic acid to uracil residues (Sheehy et al. Nature Medicine 2003 9:1404-7).

There is a need for anti-HIV agents that counteract the counteract the co-modulation of host cell processes by viral proteins. The present invention addresses this need.

Literature

Gu et al. “Good to CU” Nature 424:21-22 (2003); Simon et al. “Evidence for a newly discovered cellular anti-HIV-1 phenotype,” Nature Medicine 4:1397-1400 (1998); Sheehy et la. “Isolation of a human gene that inhibits HIV-1 infection and is suppressed by the viral Vif protein,” Nature 418:646-650 (2003); Mangeat et al. “Broad antiretroviral defence by human APOBEC3G through lethal editing of nascent reverse transcripts,” Nature 424:99-103 (2003); Harris et al “DNA deamination mediates innate immunity to retroviral infection,” Cell 113:803-809 (2003); Mariani et al. “Speces-specific exclusion of APOBEC3G from HIV-1 virions by Vif,” Cell 114:21-31 (2003); Pham et al. “Processive AID-catalysed cytosine deamination on single-stranded DNA simulates somatic hypermutation,” Nature 424:103-107 (2003); Zhang et al. “The cytidine deaminse CEM15 induces hypermutationin newly synthesized HIV-1 DNA” Nature 424:94-925 (2003); Shindo et al. “The enzymatic activity of CEM15/Apobec-3G Is essential for the regulation of the infectivity of HIV-1 Virion, but not a sole determinant of Its antiviral activity,” J. Biol. Chem. 2003 Sep. 11 [Epub ahead of print]; Lake et al. “The role of Vif during HIV-1 infection: interaction with novel host cellular factors,” J Clin Virol. 26(2):143-52. (2003); Sova et al. “Efficiency of viral DNA synthesis during infection of permissive and nonpermissive cells with Vif-negative human immunodeficiency virus type 1,” J Virol 67:6322-6366 (1993); Zhang et al. “Human immunodeficiency virus type 1 Vif protein is an integral component of an mRNP complex of viral RNA and could be involved in the viral RNA folding and packaging process,” J Virol 74:8252-8261 (2000); and Henzler et al. J Gen Virol 82:561-573 (2001); Henzler et al. “Fully functional, naturally occurring and C-terminally truncated variant human immunodeficiency virus (HIV) Vif does not bind to HIV Gag but influences intermediate filament structure,” J Gen Virol 82:561-573 (2001); Crowe et al. “The contribution of monocyte infection and trafficking to viral persistence, and maintenance of the viral reservoir in HIV infection,” J. Leukoc Biol August 21 [Epub ahead of print] (2003); Harris et al. “DNA deamination mediates innate immunity to retroviral infection,” Cell 113:803-809 (2003); Harris et al. “DNA deamination: not just a trigger for antibody diversification but also a mechanism for defense against retroviruses,” Nat. Immunol. 4:641-643 (2003); Yu et al. “Induction of APOBEC3G Ubiquitination and Degradation by an HIV-1 Vif-Cu15-SCF Complex,” Sciencexpress 16 Oct. 2003, Epub; Stopak et al. “HIV-1 Vif Blocks the Antiviral Activity of APOBEC3G by Impairing Both Its Translation and Intracellular Stability,” 2003 Molecular Cell 12:591-601; Marin et al. “HIV Vif Protein Binds the Editing Enzyme APOBEC3G and Induces its Degradation,” Nat Med. 2003 9:1398-403.

### SUMMARY OF THE INVENTION

The present invention relates to methods of inhibiting replication of a retrovirus, such as human immunodeficiency virus (HIV), by inhibiting modulation of ubiquitylation of a host cell substrate protein, such as CEM15, where ubiquitylation modulation is mediated by a retroviral protein, such as HIV Vif or Vpu. The invention also relates to screening methods for identifying agents that inhibit viral replication by inhibiting retroviral protein-mediated modulation of host cell ubiquitylation.

These and other advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic showing the consensus sequence of the retroviral Vif protein, as well as exemplary Vif amino acid sequences. From top to bottom, the sequences shown in FIG. 1 are present in the sequence listing as SEQ ID NOS:1-10, respectively.

FIG. 2 is a schematic showing the consensus sequence of the retroviral Vpu protein, as well as exemplary Vpu amino acid sequences. From top to bottom, the sequences shown in FIG. 2 are present in the sequence listing as SEQ ID NOS:11-20, respectively.

FIGS. 3A and 3B are compilation figures showing that vif is active in both Phoenix and HeLa cells containing an apobec3G (A3G)-luciferase reporter fusion protein.

FIG. 4 is a compilation figure showing increased activity of an apobec3G-luciferase fusion with MG132 in the presence of apobec3G/vif in HeLa cells.

FIGS. 5A-5E are compilation figures showing that different vectors may be employed to express vif in a cell of a subject assay.

FIG. 6 is a compilation figure showing that stable HeLa clone FD3 has high A3G-luciferase reporter activity and protein levels.

FIG. 7 is a schematic representation of a cell-free biochemical assay for detecting vif-mediated apobec3G ubiquitination.

FIG. 8 is a schematic representation of the components of the cell-free assay shown in FIG. 7 and an exemplary expression system used for their expression.

FIG. 9 is an image of a gel showing expression and purification of apobec3G-His6 from E. coli.

FIG. 10 is two panels of gels showing expression, purification, and cleavage of GST-elongin B from E. coli.

FIG. 11 is two panels of gels showing expression, purification, and cleavage of GST-elongin B from SF9 cells.

FIG. 12 is two panels of gels showing expression, purification, and cleavage of GST-elongin C from SF9 cells.

FIG. 13 is two panels of gels showing expression and purification of GST-vif from E. coli and SF9 cells.

Before the present invention is described in more detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary and preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a candidate agent” includes a plurality of such candidate agents and reference to “the host cell” includes reference to one or more host cell and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

“CEM15”, also known as APOBEC3G, apolipoprotein B mRNA editing enzyme, and catalytic polypeptide-like 3G, refers to a mammalian host cell protein, particularly a human host cell protein, which is a cytidine deaminse that induces hypermutation in newly synthesized HIV-1 DNA. CEM15 acts as a deaminase to convert C's to U's in the DNA minus strand produced from retroviral RNA during retroviral replication. When the plus strand of the CEM15-modified virus strand is produced, the plus stand contains G-to-A hypermutation.

“Ubiquitylated” or “ubiquitylation” in reference to a protein is meant to encompass proteins modified by conjugation to a ubiquitin (Ub) or a ubiquitin-like modifier (Ubl).

By “ubiquitin agents” is meant a molecule involved in ubiquitination, most frequently enzymes. Ubiquitin agents can include ubiquitin activating agents, ubiquitin ligating agents and ubiquitin conjugating agents. In addition, ubiquitin agents can include ubiquitin moieties as described below. In addition, de-ubiquitylation agents (e.g. proteases that degrade or cleave ubiquitin or polyubiquitin chains) find use in the invention.

“Vif”, also known as “virion infectivity factor”, as used herein refers to a viral protein of a retrovirus, particularly a lentivirus, e.g., a human immunodeficiency virus (HIV), simian immunodeficiency virus, or feline leukemia virus (FLV). Vif of HIV, and more particularly of HIV type 1 (HIV-1), are of particular interest. Vif enhances retroviral replication in a host cell by interfering with host cell antiviral activity mediated by CEM15.

“Vpu”, also known as “virus protein U”, refers to a retroviral protein, particularly a lentivirus, e.g., a human immunodeficiency virus (HIV), simian immunodeficiency virus, or feline leukemia virus (FLV). Vpu of HIV, and more particularly of HIV type 1 (HIV-1), are of particular interest. Vpu protein stimulates virus production by enhancing the release of viral particles from infected cells. Vpu protein binds specifically to CD4. Vpu also targets CD4 for proteasomal degradation by recruiting the SCF^{βTrCp} ubiquitin E3 ligase complex to the cytoplasmic tail of CD4.

“TRAC-1” as used herein refers to an E3 ubiquitin ligase as described in PCT Publication No. WO 02/081730, published Oct. 17, 2002, which publication is incorporated herein in its entirety.

“USP-25” as used herein refers to a de-ubiquitylating agent or de-ubiquitylating enzyme (or “DUB”) as described in U.S. Patent Application Publication Nos. US 2003/0036107 (published Feb. 23, 2003) and US 2003/0092605 (published May 15, 2003), each of which publications are incorporated herein in their entireties.

“Assay components” as used herein generally comprise, in one embodiment, at least a ubiquitin moiety, a ubiquitin activating agent, a ubiquitin conjugating agent, a ubiquitin ligating agent, a ubiquitin substrate protein, and a retroviral ubiquitylation modulator protein, and, optionally a de-ubiquitylation agent. In another embodiment, the assay components generally comprise, a ubiquitin moiety, a ubiquitylated substrate protein, and a retroviral ubiquitylation modulator protein. In the methods of the invention, the assay components are combined with a candidate agent to assess the effect of the candidate agent upon ubiquitylation and/or de-ubiquitylation activity. In some embodiments, the assay components may be present in a cell.

“Isolated” means that the recited material is unaccompanied by at least some of the material with which it is normally associated in its natural state, preferably constituting at least about 0.5%, more preferably at least about 5% by weight of the total protein in a given sample. “Purified” means that the recited material comprises at least about 75% by weight of the total protein, with at least about 80% being preferred, and at least about 90% being particularly preferred.

The terms “polypeptide” and “protein” are used interchangeably throughout the application and mean at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. Normally, the amino acids are in the (S) or L-configuration. If non-naturally occurring side chains are used, non-amino acid substituents may be used, for example to prevent or retard in vivo degradation. Naturally occurring amino acids are normally used and the protein is a cellular protein that is either endogenous or expressed recombinantly.

A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes, but is not limited to, the production of a protein from one organism in a different organism or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed below.

By “nucleic acid” herein is meant either DNA or RNA, or molecules which contain both deoxy- and ribonucleotides. The nucleic acids include genomic DNA, cDNA and oligonucleotides including sense and anti-sense nucleic acids. Also siRNA are included. Such nucleic acids may also contain modifications in the ribose-phosphate backbone to increase stability and half life of such molecules in physiological environments.

The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Other definitions of terms appear throughout the specification.

### DETAILED DESCRIPTION OF THE INVENTION

The invention focuses upon identification of antiviral agents that inhibit retroviral replication by modulating ubiquitylation of a host cell protein having antiviral activity (e.g., a host cell protein that can decrease the permissiveness of a cell to viral replication). In particular, the invention features methods for identifying agents that enhance a level of un-ubiquitylated CEM15 in retroviral virions released from an infected host cell; agents that enhance a level of un-ubiquitylated CEM15 in a host cell infected with a retrovirus, such as HIV, particularly HIV-1. The invention also particularly features methods for identifying agents that enhance a level of un-ubiquitylated CD4 on a host cell membrane in a host cell infected with a retrovirus, such as HIV, particularly HIV-1. The agents so identified counteract in some manner the activity of retroviral ubiquitylation modulator proteins, such as Vif and Vpu of HIV, in modulation of ubiquitylation of host cell substrate proteins, e.g., CEM15, CD4.

The invention further features methods of inhibiting retroviral replication by administration of such antiviral agents, and particularly in inhibiting ubiquitylation of CEM15 facilitated by E1 or by TRAC-1 or other E3s, or in enhancing de-ubiquitylation of CEM15 by USP-25 or other deubiquitylation factors.

CEM15 is a host cell protein which exhibits deaminase to convert C's to U's in the DNA minus strand produced from retroviral RNA during retroviral replication. When the plus strand of the CEM15-modified virus strand is produced, the plus stand contains G-to-A hypermutation, which can hamper replication of the virus (e.g., due to insertion of stop codons, and the like). See, e.g., Harris et al. Nat. Immunol. 4:641-3.

Screening Methods of the Invention

In one aspect, the invention features screening methods for, for example, identification of agents that modulate ubiquitylation of a host cell substrate protein, such as CEM15, in the presence of a retroviral protein, such as Vif or Vpu. Components useful in the assay are described below, and then various exemplary assay formats are provided.

Assay Components Useful in the Methods of the Invention

The following section describes the various components that can be present in the screening assays of the invention. As noted above, “ubiquitin agents’ as used herein refers to a collection of proteins that facilitates transfer, attachment, or removal of a ubiquitin moiety to or from a target protein. In this case the target protein of interest is the host protein CEM15. In each of the assays described herein (except for control assays), the retroviral ubiquitylation modulator protein can be, for example, at least one of a retroviral Vif or Vpu protein.

Examples of ubiquitin agents include ubiquitin activating agents, ubiquitin conjugating agents, and ubiquitin ligating agents. In particular embodiments, the ubiquitin activating agent is preferably an E1 or a variant thereof; the ubiquitin conjugating agent is preferably an E2 or a variant thereof; and the ubiquitin ligating agent is preferably an E3 or variant thereof.

The present invention provides methods of assaying for agents that, in the presence of a retroviral ubiquitylation modulator protein, modulate the attachment of a ubiquitin moiety to a ubiquitin agent, target protein, or mono- or poly-ubiquitin moiety of a ubiquitin agent or target protein.

Ubiquitin Moieties

By “ubiquitin moiety” herein is meant a polypeptide which is transferred or attached to another polypeptide by a ubiquitin agent. Ubiquitin moiety includes both ubiquitin and ubiquitin-like molecules, also know as “ubiquitin-like modifiers”. In preferred embodiments the ubiquitin moiety comprises a mammalian ubiquitin, and more preferably a human ubiquitin.

By “ubiquitin” or “ubiquitin moiety” is meant a polypeptide which is transferred or attached to another polypeptide by a ubiquitin agent. Ubiquitin as used in the assays below can be from any species of organism, preferably a eukaryotic species. In preferred embodiments the ubiquitin comprises a mammalian ubiquitin, and more preferably a human ubiquitin. In one embodiment the ubiquitin moiety is ubiquitin, which can comprises a 76 amino acid human ubiquitin, such as that in ATCC accession No. P02248, available in GenBank, which sequence is:

1 mqifvktltg ktitleveps dtienvkaki qdkegippdq qrlifagkql edgrtlsdyn
61 iqkestlhlv lrlrgg

In other embodiments, the ubiquitin moiety comprises ubiquitin-like molecules having an amino acid sequence or nucleic acid sequence of a sequence corresponding to one of the GENBANK accession numbers disclosed in Table 1A. Other embodiments utilize variants of ubiquitin, as further described below.

TABLE 1A
UBIQUITIN-LIKE MOLECULES

| | | GenBank | GenBank |
|---|---|---|---|
| | | Nucleotide | Protein |
| Common | | Accession | Accession |
| Name | Alias | Number | Number |
| Ubiquitin | | NM_002954.2 | NP_002945 |
| NEDD8 | RUB1 | NM_006156.1 | NP_006147 |
| ISG-15 | UCRP | NM_005101.1 | NP_005092.1 |
| APG12 | APG12L, | NM_004707.1 | NP_004698.1 |
| | MAP1_LC3 | | |
| APG8 | MAP1_LC3, | NM_022818.2 | NP_073729.1 |
| | MAP1A, 1BLC3 | | |
| Fat10 | Diubiquitin | NM_006398.1 | NP_006389.1 |
| Fau, Fubi | FBR-MuSV-associated | NM_001997.2 | NP_001988.1 |
| | ubiquitously | | |
| | expressed gene, | | |
| | ubiquitin-like | | |
| | protein fubi, | | |
| | 40S ribosomal | | |
| | protein S30, FAU- | | |
| | encoded ubiquitin- | | |
| | like protein | | |
| SUMO-1 | Sentrin1, SMT3C, | NM_003352.2 | NP_003343.1 |
| | GMP1, PIC, SM, | | |
| | SMT3H3 | | |
| SUMO-2 | Sentrin3, | NM_006936.1 | NP_008867.1 |
| | SMT3A, SMT3H1 | | |
| SUMO-3 | Sentrin2, | NM_006937.2 | NP_008868.2 |
| | SMT3B, SMT3H2, | | |
| | HSMT3 | | |

As used herein, “poly-ubiquitin moiety” refers to a chain of ubiquitin moieties comprising more than one ubiquitin moiety. As used herein, “mono-ubiquitin moiety” refers to a single ubiquitin moiety. In the methods of the present invention, an un-ubiquitylated protein, or a mono- or poly-ubiquitylated protein can serve as a substrate molecule for the transfer or attachment of a ubiquitin moiety (which can itself be a mono- or poly-ubiquitin moiety).

In an embodiment of particular interest, when one or more ubiquitin moieties are attached to a target protein, that protein is targeted for degradation by the 26S proteasome. A target protein of interest in the present invention is CEM15.

The invention also contemplates use of variants of a ubiquitin moiety which retain characteristics of the native ubiquitin moiety in being capable of being attached and/or cleaved from a target substrate protein. Such ubiquitin moiety variants generally have an overall amino acid sequence identity of preferably greater than about 75%, more preferably greater than about 80%, even more preferably greater than about 85% and most preferably greater than 90% of the amino acid sequence of ubiquitin provided above. In some embodiments the sequence identity will be as high as about 93 to 95 or 98%. Variants of ubiquitin and other components of the assays of the invention are described below in more detail.

Ubiquitin moieties of the present invention are polypeptides that may be shorter or longer than the amino acid sequence of human ubiquitin depicted above. Thus, included within the definition of ubiquitin moiety are portions or fragments of the amino acid sequence human ubiquitin. In one embodiment herein, fragments of ubiquitin moiety are considered ubiquitin moieties if they are attached to another polypeptide by a ubiquitin agent.

In addition, as is more fully outlined below, ubiquitin moieties of the present invention are polypeptides that can be made longer than the reference amino acid sequence; for example, by the addition of tags, the addition of other fusion sequences, or the elucidation of additional coding and non-coding sequences. As described below, the fusion of a ubiquitin moiety to a fluorescent peptide, such as Green Fluorescent Peptide (GFP), is of particular interest.

In one embodiment, the ubiquitin moiety is an endogenous molecule. That is, where the assay involves the use of cells, the ubiquitin moiety is naturally expressed in the cell to be assayed. However, in an alternative embodiment, the ubiquitin moiety, as well as other proteins of the present invention, are exogenous, e.g., recombinant proteins. A “recombinant protein” is a protein made using recombinant techniques, i.e. through the expression of a recombinant nucleic acid as described below. In an exemplary embodiment, the ubiquitin moiety of the invention is made through the expression of a nucleic acid sequence corresponding to GENBANK accession number M26880 or AB003730, or a fragment thereof, and preferably encodes the human ubiquitin amino acid sequence depicted above.

Ubiquitin Activating Agents

As used herein “ubiquitin activating agent” refers to a ubiquitin agent, preferably a protein (e.g., a ubiquitin activating enzyme), that transfers or attaches a ubiquitin moiety to a ubiquitin conjugating agent. Generally, the ubiquitin activating agent forms a high energy thiolester bond with ubiquitin moiety, thereby “activating” the ubiquitin moiety, and transfers or attaches the ubiquitin moiety to a ubiquitin conjugating agent (e.g., E2).

In a preferred embodiment the ubiquitin activating agent is an E1, which can transfer or attach ubiquitin to an E2, defined below. In a preferred embodiment, E1 binds ubiquitin. In a preferred embodiment, E1 forms a high energy thiolester bond with ubiquitin, thereby “activating” the ubiquitin.

In exemplary embodiments, E1 proteins useful in the invention include those having the amino acid sequence of the polypeptide having ATCC accession numbers AAA61246, P22314, and CAA40296, incorporated herein by reference. Preferably E1 is human E1. E1 is commercially available from Affiniti Research Products (Exeter, U.K.).

In further exemplary embodiments, nucleic acids which may be used for producing E1 proteins for the invention include, but are not limited to, those disclosed by GenBank accession numbers M58028 and X56976, incorporated herein by reference. Variants of the cited E1 proteins, also included in the term “E1”, can be made as described herein.

Further exemplary ubiquitin activating agents include those having the amino acid sequences or encoded by the nucleic acid sequences of a Genbank data base accession number listed in Table 1B below.

TABLE 1B

| | | ACCESSION |
|---|---|---|
| SYMBOL | DESCRIPTION | NO. |
| APPBPI | Amyloid beta precursor protein binding | NM_003905 |
| | protein 1, 59 kD | |
| FLJ23251 | hypothetical protein FLJ23251 | NM_024818 |
| GSA7 | ubiquitin activating enzyme E1-like | NM_006395 |
| | protein | |
| | similar to ubiquitin-activating enzyme | XM_088743 |
| | E1 (A1S9T and BN75 temperature sensi- | |
| | tivity complementing) (H. sapiens) | |
| | similar to SUMO-1 activating enzyme | XM_090110 |
| | subunit 1; SUMO-1 activating enzyme E1 N | |
| | subunit; sentrin/SUMO-activating protein | |
| | AOS1; ubiquitin-like protein SUMO-1 | |
| | activating enzyme | |
| SAE1 | SUMO-1 activating enzyme subunit 1 | NM_005500 |
| | | and |
| | | XM_009036 |
| UBA2 | SUMO-1 activating enzyme subunit 2 | NM_005499 |
| UBE1 | ubiquitin-activating enzyme E1 (A1S9T | NM_003334 |
| | and BN75 temperature sensitivity | and |
| | complementing) | XM_033895 |
| UBE1C | ubiquitin-activating enzyme E1C (UBA3 | NM_003968 |
| | homolog, yeast) | |
| UBE1L | Ubiquitin-activating enzyme E1-like | NM_003335 |

Further exemplary E1 proteins for use in the invention are disclosed in PCT Publication No. WO 01/75145. Variants of the cited E1 proteins, also included in the term “E1”, can be made as described herein.

The invention also contemplates use of variants of a ubiquitin activating agents which retain a characteristic of a native ubiquitin activating agent in being capable of facilitating activation of a ubiquitin conjugating agent. Such ubiquitin activating agent variants generally have an overall amino acid sequence identity of preferably greater than about 75%, more preferably greater than about 80%, even more preferably greater than about 85% and most preferably greater than 90% of the amino acid sequence of a ubiquitin provided above. In some embodiments the sequence identity will be as high as activating agent about 93 to 95 or 98%. Variants of ubiquitin activating agents and other components of the assays of the invention are described below in more detail.

Ubiquitin Conjugating Agents

As used herein “ubiquitin conjugating agent” refers to a ubiquitin agent, preferably a protein (e.g., a ubiquitin conjugating enzyme), capable of facilitating transfer or attaching a ubiquitin moiety to a substrate protein through interaction with a ubiquitin ligating agent. In some cases, the ubiquitin conjugating agent is capable of directly transferring or attaching ubiquitin moiety to lysine residues in a target substrate protein. The ubiquitin conjugating agent can be one capable of facilitating transfer or attachment of a ubiquitin moiety to a mono- or poly-ubiquitin moiety, which in turn can be attached to a ubiquitin agent or target protein.

Preferably, the ubiquitin conjugating agent is an E2, where the ubiquitin moiety is transferred from E1 to E2, in which the transfer results in a thiolester bond formed between E2 and ubiquitin moiety. In a preferred embodiment, E2 facilitates transfer or attachment of a ubiquitin moiety to a substrate protein through interaction with an E3 ubiquitin ligating agent, which is defined below.

In the methods and compositions of the present invention, the ubiquitin activating agent can comprise an amino acid sequence or a nucleic acid sequence corresponding to a sequence of an Genbank data base accession number listed in Table 2 below and incorporated herein by reference. Ubiquitin conjugating agents of human cells (indicated by “Hs”) are of particular interest.

TABLE 2

| | | Accession No. | Accession No. |
|---|---|---|---|
| | | (nucleic acid | (amino acid |
| Name | ALIAS | sequences) | sequences) |
| UBE2D1 Hs UBC4/5 | UBE2D1, UBCH5A, UBC4/5 | NM_003338.1 | NP_003329.1 |
| homolog | homolog | | |
| UBC9 Gallus gallus | UBC9, SUMO-conjugating enzyme | AB069964.1 | BAB68210.1 |
| UBC9 Mus musculus | mUB69 | U76416.1 | AAB18790.1 |
| UBC9/UBE21 Hs | UBE21 | U45328.1 | AAA86662.1 |
| UBC9 | MGC: 3994, IMAGE: 2819732, | BC004437.1 | AAH04437.1 |
| isoform/MGC: 3994 Hs | UBC9 isoform | NM_003345.1 | NP_003336.1 |
| UBC9 Hs | UBC9, UBE21 | | |
| FTS homolog Hs+ 1aa | fused toes homolog, FLJ13258 | NM_022476.1 | NP_071921.1 |
| FLJ13988 Hs | FLJ13988, clone Y79AA1002027, | AK024050.1 | BAB14800.1 |
| MGC: 13396 Hs | sim to E2-18 | BC010900.1 | AAH10900.1 |
| UBE2V2 Hs | MGC: 13396, IMAGE: 4081461 | NM_003350.2 | NP_003341.1 |
| MGC: 10481 Hs | UBE2V2, EDAF-1, MMS2, UEV2, | BC004862.1 | AAH04862.1 |
| XM_054332.1 Hs | DDVIT1, ED | XM_054332.1 | XP_054332.1 |
| FLJ13855 Hs | MGC: 10481, IMAGE: 3838157 | XM_030444.3 | XP_030444.1 |
| E2-230K homolog Hs | FLJ13855 | NM_022066.1 | NP_071349.1 |
| UBE2V2 Hs | E2-230K ortholog, FLJ12878, | NM_003339.1 | NO_003330.1 |
| UBE2D3 Hs 1 SNP | KIAA1734 | NM_003340.1 | NP_003331.1 |
| Non-canon Ub-conj Enz | UBE2D2, UBCH5B, UBC4, | NM_016336.2 | NP_057420.2 |
| (NCUBE1) | UBC4/5 homolog | NM_014176.1 | NP_054895.1 |
| HSPC150 Hs | UBE2D3, UBCH5C, UBC4/5 | NM_016252.1 | NP_057336.1 |
| Brain 1AP repeat | homolog | | |
| contain 6 (BIRC6) | NCUBE1, HSU93243, HSPC153, | | |
| | CGI-76 | | |
| | BIRC6, KIAA1289, apollon | | |
| UBC8 Mus | E2-20K, UBE2H | NM_009459.1 | NP_033485.1 |
| UBC8 Hs | UBE2H, UBCH, UBCH2, UBC8 | NM_003344.1 | NP_003335.1 |
| UBC8 Hs 6SNP | homolog | NM-003344.1 | NP-003335.1 |
| UBC8 Hs no 5′ | UBE2H, UBCH, UBCH2, UBC8 | | |
| | homolog | | |
| RAD6 homolog Hs | UBE2B, RAD6B, HHR6B, UBC2, | NM_003337.1 | NP_003328.1 |
| | RAD6 homolog | | |
| UBE2V1 var 3 Hs | UBE2V1, CIR1, UEV1, UEV1A, | NM_022442.2 | NP_071887.1 |
| UBE2V1 var 1 Hs early | CROC-1, CRO | NM_021988.2 | NP_068823.1 |
| stop, 56aa | UBE2V1, CIR1, UEV1, UEV1A, | NM_003349.3 | NP_003340.1 |
| UBE2V1 var 2 Hs | CROC-1, CRO | | |
| | UBE2V1, CIR1, UEV1, UEV1A, | | |
| | CROC-1, CRO | | |
| UBE2L6 Hs | UBE2L6, UBCH8, RIG-B | NM_004223.1 | NP_004214.1 |
| UBE2L3 Hs 2 SNP | UBE2L3, UBCH7 | NM_003347.1 | NP_003338.1 |
| UBE2E1 Hs | UBE2E1, UBCH6, UBC4/5 | NM_003341.1 | NP_003332.1 |
| RAD6/UBE2A Hs | homolog | NM_003336.1 | NP_003327.1 |
| UBE2E3 Hs | UBE2A, RAD6A, HHR6A, UBC2, | NM_006357.1 | NP_006348.1 |
| UBC12/UBE2M Hs | RAD6 homolog | NM_003969.1 | NP_003960.1 |
| UBC7/UBE2G1 Hs | UBE2E3, UBCH9, UBC4/5 | NM_003342.1 | NP_003333.1 |
| | homolog | | |
| | UBE2M, HUBC12, UBC12 | | |
| | homolog | | |
| | UBE2G1, UBC7 homolog | | |
| Huntingtin interact prot | HIP2, LIG, E2-25K | NM_005339.2 | NP_005330.1 |
| 2 (HIP2) Hs | LIG, HIP2 alternative splicing form | ABO22436.1 | BAA78556.1 |
| LIG/HIP2 variant Hs | | | |
| UBC6p Hs | UBC6p, UBC6 | NM_058167.1 | NP_477515.1 |
| UBC6 Hs | UBC6 | AF296658.1 | AAK52609.1 |
| HBUCE1/UBE2D2 var | HBUCE1, LOC51619 | NM_015983.1 | NP_057067.1 |
| Hs | UBE2G2, UBC7 homolog | XM_036087.1 | XP_036087.1 |
| UBE2G2/UBC7 | NCE2 | NM_080678.1 | NP_542409.1 |
| homolog Hs | CDC34, E2-CDC34, E2-32 | NM_004359.1 | NP_004350.1 |
| NEDD8-conj enzyme 2 | complementing | BC000848.1 | AAH00848.1 |
| (NCE2) Hs | IMAGE: 3458173 | | |
| CDC34 Hs | | | |
| IMAGE: 3458173/NICE- | | | |
| 5 var | | | |
| UBE2C Hs | UBE2C, UBCH10 | NM_007019.1 | NP_008950.1 |
| UBE2C possible short | UBE2C, UBCH10 | NM_007019.1 | NP_008950.1 |
| form Hs | | | |
| UBC3/UBE2N Hs | UBE2N, UBCH-BEN, UBC13 | NM_003348.1 | NP_003339.1 |
| FLJ25157 Hs | hom., sim to bend | AK057886.1 | BAB71605.1 |
| TSG101 Hs 1 SNP | FLJ25157, highly similar to E2-23 | NM_006292.1 | NP_006283.1 |
| MGC: 21212/NICE-5 | Tumor susceptibility gene 101 | BC017708.1 | AAH17708.1 |
| var Hs | MCG: 21212, IMAGE: 3907760, | | |
| | sim to NICE-5 | | |

Hs = Homo sapiens;
Mm = Mus musculus;

Sequences encoding a ubiquitin conjugating agent may also be used to make variants thereof that are suitable for use in the methods and compositions of the present invention. The ubiquitin conjugating agents and variants suitable for use in the methods and compositions of the present invention may be made as described herein.

In exemplary embodiments, the E2 used in the methods and compositions of the present invention comprises an amino acid sequence or nucleic acid sequence of a sequence corresponding to an Genbank data base accession number in the following list: AC37534, P49427, CAA82525, AAA58466, AAC41750, P51669, AAA91460, AAA91461, CAA63538, AAC50633, P27924, AAB36017, Q16763, AAB86433, AAC26141, CAA04156, BAA11675, Q16781, NP_{—}003333, BAB18652, AAH00468, CAC16955, CAB76865, CAB76864, NP_{—}05536, 000762, XP_{—}009804, XP_{—}009488, XP_{—}006823, XP_{—}006343, XP_{—}005934, XP_{—}002869, XP_{—}003400XP_{—}009365, XP_{—}010361, XP_{—}004699, XP_{—}004019, 014933, P27924, P50550, P52485, P51668, P51669, P49459, P37286, P23567, P56554, and CAB45853, each of which is incorporated herein by reference. Exemplary sequences of interest are those corresponding to Genbank data base accession numbers NP003331, NP003330, NP003329, P49427, AAB53362, NP008950, XP009488 and AAC41750, also incorporated by reference.

In further exemplary embodiments, E2 is one of Ubc5 (Ubch5, e.g., Ubch5c), Ubc3 (Ubch3), Ubc4 (Ubch4) and UbcX (Ubc10, Ubch10). In an exemplary embodiment, E2 is Ubc5c. In an exemplary embodiment, nucleic acids which may be used to make E2 include, but are not limited to, those nucleic acids having sequences disclosed in ATCC accession numbers L2205,229328, M92670, L40146, U393 17, U393 18, X92962, U58522, S81003, AF03 1141, AF075599, AJ000519, XM009488, NM007019, U73379, L40146 and D83004, each of which is incorporated herein by reference. As described above, variants of these and other E2 encoding nucleic acids may also be used to make variant E2 proteins.

The skilled artisan will appreciate that many different E2 proteins and isozymes are known in the field and may be used in the present invention, provided that the E2 has ubiquitin conjugating activity. Also specifically included within the term “E2” are variants of E2, which can be made as described herein.

The skilled artisan will appreciate that many different E2 proteins and isozymes are known in the field and may be used in the present invention, provided that the E2 has ubiquitin conjugating activity. Further exemplary E2 proteins for use in the invention are disclosed in PCT Publication No. WO 01/75145. Also specifically included within the term “E2” are variants of E2, which can be made as described herein.

The invention contemplates use of variants of a ubiquitin conjugating agents which retain a characteristic of a native ubiquitin conjugating agent in being capable of being activated by a ubiquitin activating agent and/or facilitating ubiquitylation of a target substrate protein in connection with a ubiquitin ligating agent. Such ubiquitin conjugating agent variants generally have an overall amino acid sequence identity of preferably greater than about 75%, more preferably greater than about 80%, even more preferably greater than about 85% and most preferably greater than 90% of the amino acid sequence of a ubiquitin conjugating agent provided above. In some embodiments the sequence identity will be as high as about 93 to 95 or 98%. Variants of ubiquitin conjugating agents and other components of the assays of the invention are described below in more detail.

In some embodiments, E2 has a tag, as defined herein, with the complex being referred to herein as “tag-E2”. Exemplary E2 tags include, but are not limited to, labels, partners of binding pairs and substrate binding elements. In one embodiment of particular interest, the tag is a His-tag or GST-tag.

Ubiquitin Ligating Agents

In some embodiments, the methods of the present invention comprise the use of a ubiquitin ligating agent. As used herein “ubiquitin ligating agent” refers to a ubiquitin agent, preferably a protein (e.g., a ubiquitin ligating enzyme), capable of facilitating transfer or attachment of a ubiquitin moiety from a ubiquitin conjugating agent to a target substrate molecule. In a preferred embodiment, the ubiquitin ligating agent is an E3.

As used herein “E3” refers to a ubiquitin ligating agent comprising one or more subunits, preferably polypeptides, associated with the activity of E3 as a ubiquitin ligating agent (i.e., associated with mediating the ligation or attachment of ubiquitin moiety to a target substrate protein).

In one embodiment of particular interest, the E3 is TRAC-1. TRAC-1 is descried in PCT Publication No. WO 02/081730, which is specifically incorporated herein by reference in its entirety.

In exemplary embodiments, E3 is a member of the HECT domain E3 ligating agents. In further exemplary embodiments, E3 is a member of the RING finger domain E3 ligating agents. In further exemplary embodiments, E3 comprises a ring finger subunit and a Cullin subunit. Examples of RING finger polypeptides suitable for use in the methods and compositions of the present invention include, but are not limited to, ROC1, ROC2 and APC11. Examples of Cullin polypeptides suitable for use in the methods and compositions of the present invention include, but are not limited to, CUL1, CUL2, CUL3, CUL4A, CUL4B, CUL5 and APC2.

In further exemplary embodiments of the present invention, the ubiquitin ligating agent comprises an amino acid sequence or a nucleic acid sequence of a sequence corresponding to an accession number in the Genbank data base, European Molecular Biology Laboratories (EMBL) data base, or ENSEMBL data base (a joint project of the European Molecular Biology Laboratories and the Sanger Institute) in Table 3 below and incorporated herein by reference. The accession numbers from the Genbank database can be found as stated above. The accession numbers from the EMBL data base and from the ENSEMBL database are found on the world wide web at the sites supported by those organizations.

TABLE 3

| | Accession | Accession | Accession | Accession | Accession | Accession | Accession | Accession |
|---|---|---|---|---|---|---|---|---|
| Accession No. | No. | No. | No. | No. | No. | No. | No. | No. |
| AAD15547 | AAH22038 | O75485 | Q96BD4 | Q96K03 | Q96T88 | Q9BYV6 | Q9H073 | Q9H920 |
| AAF42995 | AAH22403 | O75592 | Q96BD | Q96K19 | Q99496 | Q9BZX6 | Q9H083 | Q9H9B0 |
| AAF91315 | AAH22510 | O75598 | 5Q96BE6 | Q96K21 | Q99579 | Q9BZX7 | Q9H0A6 | Q9H9B5 |
| AAF97687 | AAL30771 | O75615 | Q96BH1 | Q96KD9 | Q99675 | Q9BZX8 | Q9H0M8 | Q9H9P5 |
| AAG50176 | AAL31641 | O75866 | Q96BL1 | Q96KL0 | Q99942 | Q9BZX9 | Q9H0V6 | Q9H9T2 |
| AAG50180 | AAL36460 | O76050 | Q96BM5 | Q96KM9 | Q9BPW2 | Q9BZY0 | Q9H0X6 | Q9H9V4 |
| AAG53500 | AAL40179 | O76064 | Q96BQ3 | Q96LD4 | Q9BQ47 | Q9BZY1 | Q9H270 | Q9H9Y7 |
| AAG53509 | AAL40180 | O94896 | Q96BS3 | Q96M70 | Q9BQV0 | Q9BZY2 | Q9H2A8 | Q9HA51 |
| AAH00832 | AAL76101 | O94941 | Q96BX2 | Q96MJ7 | Q9BRZ2 | Q9BZY3 | Q9H2S3 | Q9HAC1 |
| AAH02922 | CAC81706 | O94972 | Q96C24 | Q96MT1 | Q9BS04 | Q9BZY4 | Q9H2S4 | Q9HAM2 |
| AAH04978 | CAC85986 | O95159 | Q96CA5 | Q96MX5 | Q9BSE9 | Q9BZY5 | Q9H2S5 | Q9HAP7 |
| AAH05375 | CAD19102 | O95247 | Q96CC2 | Q96MZ7 | Q9BSL8 | Q9BZY6 | Q9H348 | Q9HBD2 |
| AAH13580 | O00237 | O95277 | Q96D24 | Q96NI4 | Q9BSM1 | Q9BZY8 | Q9H463 | Q9HCL8 |
| AAH15738 | O00463 | O95604 | Q96D38 | Q96NS4 | Q9BSV9 | Q9BZY9 | Q9H4C2 | Q9HCR0 |
| AAH16174 | O00635 | O95627 | Q96D59 | Q96NT2 | KIAA066 | Q9C017 | Q9H4C3 | Q9HCR1 |
| AAH16924 | O14616 | O95628 | Q96DB4 | Q96P09 | Q9BTC5 | Q9C018 | Q9H4C4 | Q9HCR2 |
| AAH17370 | O14686 | O96028 | Q96DV2 | Q96PF7 | Q9BTD9 | Q9C019 | Q9H4C5 | Q9HCS6 |
| AAH17585 | O15057 | Q14527 | Q96DV3 | Q96PH3 | Q9BU73 | Q9C021 | Q9H4J2 | Q9NPN4 |
| AAH17592 | O15262 | Q14536 | Q96DX4 | Q96PK3 | Q9BUW4 | Q9C025 | Q9H5E4 | Q9NPP8 |
| AAH17707 | O15344 | Q14848 | Q96DY5 | Q96PM5 | Q9BUZ4 | Q9C026 | Q9H5F1 | Q9NPQ1 |
| AAH18104 | O43164 | Q15156 | Q96EL5 | Q96PR5 | Q9BV68 | Q9C027 | Q9H5K0 | Q9NQ86 |
| AAH18107 | O43255 | Q15290 | Q96EP1 | Q96PU4 | Q9BVG3 | Q9C029 | Q9H5L8 | Q9NQP8 |
| AAH18198 | O43269 | Q15521 | Q96EP8 | Q96PX1 | Q9BW41 | Q9C030 | Q9H5P2 | Q9NR13 |
| AAH18337 | O43270 | Q15959 | Q96EQ8 | Q96QB5 | Q9BW90 | Q9C031 | Q9H5S6 | Q9NRL2 |
| AAH18647 | O43567 | Q16030 | Q96F06 | Q96QB6 | Q9BWF2 | Q9C032 | Q9H647 | Q9NRT4 |
| AAH19283 | O60272 | Q92550 | Q96F37 | Q96QY9 | Q9BWL5 | Q9C033 | Q9H6D9 | Q9NRT6 |
| AAH19355 | O60291 | Q92897 | Q96F67 | Q96RF3 | Q9BWP7 | Q9C034 | Q9H6S6 | Q9NS55 |
| AAH20556 | O60372 | Q969K3 | Q96GF1 | Q96RF8 | Q9BX37 | Q9C035 | Q9H6W8 | Q9NS56 |
| AAH20964 | O60630 | Q969Q1 | Q96GT5 | Q96RW5 | Q9BXI1 | Q9C036 | Q9H6Y7 | Q9NS56 |
| AAH20984 | O75150 | Q969V5 | Q96H69 | Q96SH4 | Q9BY78 | Q9C037 | Q9H748 | Q9NS91 |
| AAH20994 | KIAA0661 | Q96A37 | Q96IB6 | Q96SJ1 | Q9BYE7 | Q9C038 | Q9H874 | Q9NSR1 |
| AAH21258 | O75162 | Q96A61 | Q96ID9 | Q96SL3 | Q9BYV2 | Q9C039 | Q9H890 | Q9NSX7 |
| AAH21570 | O75188 | Q96AK4 | Q96J90 | Q96SR5 | Q9BYV3 | Q9C040 | Q9H8K2 | Q9NTX6 |
| AAH21571 | O75341 | Q96AX9 | Q96JD3 | Q96T06 | Q9BYV4 | Q9C0B0 | Q9H8V9 | Q9NTX7 |
| AAH21925 | O75382 | Q96BD3 | Q96JL5 | Q96T18 | Q9BYV5 | Q9C0G7 | Q9H8W5 | Q9NU68 |

| Accession | Accession | Accession | Accession | Accession | Accession | Accession | Accession |
|---|---|---|---|---|---|---|---|
| No. | No. | No. | No. | No. | No. | No. | No. |
| Q9NUH2 | Q9NZS9 | Q9UIG0 | 9UQPQ7 | O15151 | Q9BXT8 | O94822 | Q13263 |
| Q9NUR4 | Q9NZT8 | Q9UIG1 | Q9UPR2 | O15541 | Q9BYM8 | O95376 | Q13489 |
| Q9NUW5 | Q9P0J9 | Q9UJ97 | Q9UQ11 | O60858 | Q9BZR9 | P15918 | Q13490 |
| Q9NVD5 | Q9P0P0 | Q9UJJ8 | Q9Y225 | O75678 | Q9H000 | P19474 | Q13702 |
| Q9NVP6 | Q9P115 | Q9UJL3 | Q9Y254 | P14373 | Q9NS80 | P22681 | Q14839 |
| Q9NW38 | Q9P1Y6 | Q9UJR9 | Q9Y2E6 | P28328 | Q9NV58 | P29590 | Q15326 |
| Q9NWD2 | Q9P200 | Q9UJV3 | Q9Y2N1 | P35226 | Q9UDY6 | P35227 | Q92785 |
| Q9NWX1 | Q9P2G1 | Q9UKI6 | Q9Y3C5 | P46100 | Q9UHC7 | P36406 | Q99728 |
| Q9NX39 | Q9P2L3 | Q9UKV5 | Q9Y3V1 | P51948 | Q9ULX5 | P38398 | Q9HCM9 |
| Q9NXC0 | Q9P2M3 | Q9ULK6 | Q9Y3V3 | Q12899 | Q9UMT8 | P49754 | Q9NVW2 |
| Q9NXD0 | Q9UBF6 | Q9ULT6 | Q9Y4I0 | Q12933 | Q9Y4X5 | P50876 | Q9NYG5 |
| Q9NXI6 | Q9UDN7 | Q9ULW4 | Q9Y4K3 | Q12986 | Q9Y508 | P53804 | Q9ULV8 |
| Q9NZ15 | Q9UEK4 | Q9UMH1 | Q9Y4L5 | Q13049 | O00623 | P98170 | Q9UPN9 |
| Q9NZB4 | Q9UF32 | Q9UMQ2 | Q9Y577 | Q13054 | O15164 | Q06587 | Q9Y252 |
| Q9NZE3 | Q9UHE7 | Q9UNR9 | Q9Y5M7 | Q13114 | O60683 | Q12873 | |
| Q9NZE9 | Q9UHW2 | Q9UPQ2 | Q9Y6E4 | Q13434 | O75677 | Q13191 | |
| Q9NZN6 | Q9UID0 | Q9UPQ4 | Q9Y6U1 | Q14258 | O75679 | Q13233 | |

TABLE 3

| | | | | |
|---|---|---|---|---|
| Hect domain | Ringfinger domain | | | |
| proteins (Embl data | proteins | | | |
| base) | (GenBank data | | | |
| | base) | | | |
| AAH19105 | AAF50078 | T14346 | BAB23311 | AAL13848 |
| AAH19345 | AAH21525 | NP_008944 | T40821 | XP_004990 |
| AAH21144 | AAH02582 | S66562 | NP_192994 | BAB29387 |
| O00307 | NP_055486 | NP_008945 | AAF57824 | BAA92558 |
| O00308 | BAB13352 | NP_032421 | NP_080106 | AAG45422 |
| O14996 | NP_492389 | AAK33088 | T37964 | AAF36454 |
| O15029 | XP_048020 | AAL39551 | NP_035798 | AAF36455 |
| O15033 | BAB28637 | NP_175982 | BAB14280 | AAK14420 |
| O15036 O43165 | BAA20780 | AAF68076 | XP_084941 | BAA74919 |
| O43584 | T39585 | AAF68077 | AAH15380 | BAB24805 |
| O94970 | NP_060239 | AAH11571 | XP_080159 | BAB30794 |
| O95071 | T39007 | XP_052430 | AAF08298 | NP_004229 |
| O95714 | BAA92539 | AAF68079 | BAA19217 | O08759 |
| Q15386 | CAC42101 | AAH04712 | T01491 | AAH19345 |
| Q15751 | XP_083009 | T38951 | CAB92704 | NP_011374 |
| Q96BP4 | AAF79338 | BAA23711 | CAB09785 | NP_056092 |
| Q96CZ2 | NP_060382 | BAB13451 | NP_177189 | AAH21144 |
| Q96DE7 | AAH00621 | AAF46512 | XP_030186 | NP_056986 |
| Q96F34 | AAH09271 | NP_000453 | AAF61856 | B38919 |
| Q96F66 | AAC62434 | AAL29143 | XP_057408 | T38617 |
| Q96GR7 | AAF51314 | AAL27259 | Q9PUN2 | AAH06848 |
| Q96J02 | T21546 | AAF36539 | CAB99103 | NP_490834 |
| Q96PU5 | NP_188346 | BAA84697 | NP_195908 | NP_010745 |
| Q9BUI0 | AAF49328 | NP_499392 | AAH11391 | CAB95249 |
| Q9BUI6 | XP_082286 | AAF68080 | NP_012570 | |
| Q9BVR2 | NP_035020 | I83196 | AAF52899 | |
| Q9BXZ4 | NP_501120 | NP_057407 | AAF88143 | |
| Q9BY75 | NP_055636 | AAF28950 | AAF68614 | |
| Q9H0M0 | NP_003913 | XP_052223 | BAA20771 | |
| Q9H2G0 | BAB02722 | AAF68082 | BAB13419 | |
| Q9H2W4 | NP_497697 | AAF68083 | NP_011051 | |
| Q9H451 | NP_490865 | T41750 | AAH13645 | |
| Q9H783 | T14761 | AAH11658 | Q9CUN6 | |
| Q9H9E9 | AAC83345 | NP_114087 | XP_046129 | |
| Q9HCC7 | S70642 | Q05086 | A38920 | |
| Q9HCH9 | AAG53076 | T49744 | AAB47756 | |
| Q9NPL3 | CAA03915 | AAC51324 | Q92462 | |
| Q9NPS9 | XP_085770 | BAA92571 | NP_113671 | |
| Q9NT88 | CAC09387 | BAB30733 | CAA57291 | |
| Q9NWS4 | NP_055421 | NP_500283 | XP_087357 | |
| Q9NXC0 | NP_523779 | AAK28419 | AAC41731 | |
| Q9NZS4 | XP_038999 | NP_446441 | BAB69424 | |
| Q9P0A9 | AAD51453 | BAA86445 | T37900 | |
| Q9P2L3 | AAB49301 | NP_190877 | T14317 | |
| Q9P2M6 | T49799 | Q9HCE7 | P51593 | |
| Q9P2P5 | AAG16783 | AAF50332 | AAH04085 | |
| Q9UDU3 | NP_195572 | AAH09527 | BAA21482 | |
| Q9UFZ7 | AAH21470 | NP_490750 | NP_012915 | |
| Q9UII4 | NP_078878 | XP_003492 | AAF48495 | |
| Q9ULT8 | NP_073576 | T37736 | XP_045232 | |
| Q9Y4D8 | XP_028151 | AAF47474 | AAF50913 | |
| Q9HAU4 | P46934 | AAD34642 | T00390 | |
| Q9HCE7 | BAB28001 | | NP_476753 | |
| P46934 | NP_004658 | | T46412 | |
| Q05086 | P46935 | | XP_045095 | |
| Q14669 | NP_524296 | | NP_113584 | |
| Q15034 | | | NP_495842 | |
| | | | AAC04845 | |
| | | | XP_030175 | |
| | | | 1C4Z | |

| | | | |
|---|---|---|---|
| Ringfinger domain | ENSP00000282135 | ENSP00000255977 | ENSP00000265742 |
| proteins (Ensembl | ENSP00000280460 | ENSP00000283460 | ENSP00000269475 |
| data base) | ENSP00000280461 | ENSP00000262370 | ENSP00000265290 |
| ENSP00000259945 | ENSP00000217740 | ENSP00000253024 | ENSP00000222597 |
| ENSP00000254436 | ENSP00000227588 | ENSP00000282369 | ENSP00000292307 |
| ENSP00000066988 | ENSP00000259944 | ENSP00000253571 | ENSP00000265267 |
| ENSP00000275736 | ENSP00000279757 | ENSP00000288913 | ENSP00000263220 |
| ENSP00000275735 | ENSP00000274773 | ENSP00000288918 | ENSP00000216225 |
| ENSP00000203439 | ENSP00000276311 | ENSP00000276573 | ENSP00000293538 |
| ENSP00000013772 | ENSP00000166144 | ENSP00000237308 | ENSP00000229766 |
| ENSP00000225283 | ENSP00000292363 | ENSP00000238203 | ENSP00000242239 |
| ENSP00000246907 | ENSP00000264616 | ENSP00000227451 | ENSP00000274616 |
| ENSP00000225285 | ENSP00000272390 | ENSP00000244360 | ENSP00000286773 |
| ENSP00000225286 | ENSP00000272396 | ENSP00000244359 | ENSP00000273480 |
| ENSP00000230239 | ENSP00000264767 | ENSP00000281105 | ENSP00000217173 |
| ENSP00000286909 | ENSP00000255499 | ENSP00000268907 | ENSP00000290337 |
| ENSP00000286910 | ENSP00000264614 | ENSP00000292962 | ENSP00000281930 |
| ENSP00000280609 | ENSP00000262482 | ENSP00000280804 | ENSP00000257575 |
| ENSP00000263651 | ENSP00000261481 | ENSP00000287546 | ENSP00000287212 |
| ENSP00000261395 | ENSP00000261658 | ENSP00000248980 | ENSP00000290788 |
| ENSP00000277584 | ENSP00000288774 | ENSP00000287559 | ENSP00000282455 |
| ENSP00000224833 | ENSP00000261675 | ENSP00000264926 | ENSP00000254247 |
| ENSP00000254604 | ENSP00000266880 | ENSP00000261737 | ENSP00000290649 |
| ENSP00000240395 | ENSP00000243674 | ENSP00000170447 | ENSP00000274542 |
| ENSP00000240318 | ENSP00000284638 | ENSP00000270944 | ENSP00000224944 |
| ENSP00000286945 | ENSP00000247668 | ENSP00000289726 | ENSP00000281418 |
| ENSP00000281874 | ENSP00000285317 | ENSP00000230099 | ENSP00000289883 |
| ENSP00000240802 | ENSP00000278480 | ENSP00000237455 | ENSP00000255325 |
| ENSP00000267825 | ENSP00000240159 | ENSP00000263550 | ENSP00000255326 |
| ENSP00000254586 | ENSP00000294256 | ENSP00000264198 | ENSP00000292543 |
| ENSP00000293123 | ENSP00000279766 | ENSP00000263464 | ENSP00000277534 |
| ENSP00000285805 | ENSP00000288204 | ENSP00000259604 | ENSP00000260947 |
| ENSP00000257633 | ENSP00000269439 | ENSP00000265673 | ENSP00000278455 |
| ENSP00000266119 | ENSP00000268061 | ENSP00000248983 | ENSP00000278454 |
| ENSP00000233630 | ENSP00000268058 | ENSP00000269391 | ENSP00000274694 |
| ENSP00000264033 | ENSP00000268059 | ENSP00000249007 | ENSP00000217740 |
| ENSP00000275619 | ENSP00000268060 | ENSP00000242719 | ENSP00000262952 |
| ENSP00000275637 | ENSP00000261825 | ENSP00000217169 | ENSP00000268154 |
| ENSP00000280063 | ENSP00000288587 | ENSP00000253642 | ENSP00000265756 |
| ENSP00000276333 | ENSP00000275693 | ENSP00000227758 | ENSP00000277490 |
| ENSP00000263651 | ENSP00000244061 | ENSP00000291190 | ENSP00000266625 |
| ENSP00000278302 | ENSP00000272598 | ENSP00000261537 | ENSP00000266624 |
| ENSP00000264122 | ENSP00000289818 | ENSP00000291733 | ENSP00000258147 |
| ENSP00000284559 | ENSP00000238349 | ENSP00000274782 | ENSP00000258148 |
| ENSP00000266252 | ENSP00000280266 | ENSP00000271287 | ENSP00000258149 |
| ENSP00000278350 | ENSP00000242855 | ENSP00000261445 | ENSP00000264512 |
| ENSP00000259847 | ENSP00000276688 | ENSP00000245836 | ENSP00000261212 |
| ENSP00000274855 | ENSP00000280268 | ENSP00000267291 | ENSP00000262642 |
| ENSP00000259930 | ENSP00000274811 | ENSP00000292195 | ENSP00000264359 |
| ENSP00000217214 | ENSP00000268363 | ENSP00000216420 | ENSP00000217537 |
| ENSP00000283330 | ENSP00000274828 | ENSP00000261464 | ENSP00000264777 |
| ENSP00000263535 | ENSP00000235150 | ENSP00000260076 | ENSP00000287880 |
| ENSP00000291416 | ENSP00000211960 | ENSP00000284244 | ENSP00000272674 |
| ENSP00000291414 | ENSP00000262843 | ENSP00000292545 | ENSP00000272662 |
| ENSP00000253769 | ENSP00000266952 | ENSP00000242669 | ENSP00000293245 |
| ENSP00000274786 | ENSP00000288300 | ENSP00000288848 | ENSP00000283875 |
| ENSP00000289896 | ENSP00000291134 | ENSP00000261809 | ENSP00000262642 |
| ENSP00000289898 | ENSP00000261947 | ENSP00000262952 | ENSP00000259865 |
| ENSP00000265771 | ENSP00000288715 | ENSP00000245937 | ENSP00000217908 |
| ENSP00000229866 | ENSP00000222704 | ENSP00000275970 | ENSP00000255004 |
| ENSP00000286475 | ENSP00000293938 | ENSP00000238647 | ENSP00000275184 |
| ENSP00000256257 | ENSP00000266030 | ENSP00000268850 | ENSP00000275183 |
| ENSP00000253554 | ENSP00000287335 | ENSP00000291963 | ENSP00000200457 |
| ENSP00000259654 | ENSP00000256649 | ENSP00000286349 | ENSP00000261537 |
| ENSP00000280266 | ENSP00000249240 | ENSP00000257600 | ENSP00000257100 |
| ENSP00000259941 | ENSP00000253953 | ENSP00000281843 | ENSP00000286349 |
| ENSP00000259940 | ENSP00000267073 | ENSP00000261245 | ENSP00000252445 |
| ENSP00000270086 | ENSP00000271813 | ENSP00000245888 | ENSP00000294213 |
| ENSP00000289140 | ENSP00000248492 | ENSP00000222704 | ENSP00000259939 |
| ENSP00000225507 | ENSP00000265981 | ENSP00000245419 | ENSP00000236892 |
| ENSP00000261593 | ENSP00000270280 | ENSP00000272023 | ENSP00000238001 |
| ENSP00000257847 | ENSP00000270279 | ENSP00000274068 | ENSP00000274657 |
| ENSP00000262881 | ENSP00000254959 | ENSP00000275233 | ENSP00000274799 |
| ENSP00000222033 | | | |
| ENSP00000290048 | | | |
| ENSP00000274327 | | | |

Sequences encoding a ubiquitin activating agent may also be used to make variants thereof that are suitable for use in the methods and compositions of the present invention. The ubiquitin ligating agents and variants suitable for use in the methods and compositions of the present invention may be made as described herein.

In one embodiment, RING finger subunits include, but are not limited to, polypeptides having an amino acid sequence corresponding to Genbank accession numbers AAD30147, AAD30146, or 6320196, incorporated herein by reference.

In further embodiments, Cullins include, but are not limited to, polypeptides having an amino acid sequence corresponding to Genbank accession number 4503161, AAC50544, AAC36681, 4503163, AAC51190, AAD23581, 4503165, AAC36304, AAC36682, AAD45191, AAC50548, Q13620, 4503167, or AAF05751, each of which is incorporated herein by reference. In addition, in the context of the invention, each of the RING finger proteins and Cullins encompass variants of the known or listed sequences, as described herein.

These E3 ligating agents and variants may be made as described herein. In exemplary embodiments, nucleic acids used to make the RING finger proteins include, but are not limited to, those having the nucleic acid sequences disclosed in Genbank accession numbers AF142059, AF142060 and nucleic acids 433493 to 433990 of NC 001136. In one embodiment, Cullins are made from nucleic acids including, but not limited to, those having nucleic acid sequences disclosed in Genbank accession numbers NM 003592, U58087, AF062536, AF126404, NM 003591, U83410, NM 003590, AB014517, AF062537, AF064087, AF077188, U58091, NM 003478, X81882 and AF 191337, each of which is incorporated herein by reference. As described herein, variants of these sequences are also encompassed by the invention.

In further exemplary embodiments, E3 comprises the RING finger protein/Cullin combination APC11/APC2. In another exemplary embodiment, E3 comprises the RING finger protein/Cullin combination ROC1/CUL1. In a further exemplary embodiment, E3 comprises the RING finger protein/Cullin combination ROC1/CUL2. In still another exemplary embodiment, E3 comprises the RING finger protein/Cullin combination ROC2/CUL5. However, the skilled artisan will appreciate that any combination of E3 components may be produced and used in the invention described herein.

In an alternate embodiment, E3 comprises the ligase E3-alpha, E3A (E6-AP), HERC2, SMURF 1, TRAF6, Mdm2, Cb1, Sina/Siah, Itchy, IAP or NEDD-4. In this embodiment, the ligase has the amino acid sequence of that disclosed in Genbank accession number AAC39845, Q05086, CAA66655, CAA66654, CAA66656, AAD08657, NP_{—}002383, XP_{—}006284, AAC51970, XP_{—}013050, BAB39389, Q00987, AAF08298 or P46934, each of which is incorporated herein by reference. As above, variants are also encompassed by the invention. Nucleic acids for making E3 for this embodiment include, but are not limited to, those having the sequences disclosed in Genbank accession numbers AF061556, XM006284, U76247, XM013050, X898032, X98031, X98033, AF071172, Z12020, AB056663, AF199364 and D42055 and variants thereof.

E3 may also comprise other components, such as SKP1 and F-box proteins. The amino acid and nucleic acid sequences for SKP1 correspond to GENBANK accession numbers AAC50241 and U33760, respectively. Many F-box proteins are known in the art and their amino acid and nucleic acid sequences are readily obtained by the skilled artisan from various published sources.

“E3” further includes variants of E3 that retain an activity of E3. In an exemplary embodiment, the E3 components are produced recombinantly, as described herein. In one embodiment, the E3 components are co-expressed in the same host cell. Co-expression may be achieved by transforming the cell with a vector comprising nucleic acids encoding two or more of the E3 components, or by transforming the host cell with separate vectors, each comprising a single component of the desired E3 protein complex. In one embodiment, the RING finger protein and Cullin are expressed in a single host transfected with two vectors, each comprising nucleic acid encoding one or the other polypeptide.

The invention contemplates use of variants of a ubiquitin ligating agent which retain a characteristic of a native ubiquitin ligating agent in being capable of facilitating transfer or attachment of a ubiquitin moiety to a target substrate protein in connection with a ubiquitin conjugating agent. Such ubiquitin ligating agent variants generally have an overall amino acid sequence identity of preferably greater than about 75%, more preferably greater than about 80%, even more preferably greater than about 85% and most preferably greater than 90% of an amino acid sequence of a ubiquitin ligating agent provided above. In some embodiments the sequence identity will be as high as about 93% to 95% or 98%. Variants of ubiquitin ligating agents and other components of the assays of the invention are described below in more detail.

De-Ubiquitylating Agents

A “de-ubiquitylating agent” refers to a ubiquitin agent, preferably a protein (e.g., a de-ubiquitylating enzyme or “DUB”), capable of removing a ubiquitin moiety from a ubiquitylated substrate protein. Preferably, the de-ubiquitylating agent is a de-ubiquitylating enzyme or “DUB”.

As used herein “DUB” refers to a de-ubiquitylating agent which can comprise one or more subunits, preferably polypeptides, associated with the activity of DUB as a de-ubiquitylating agent (i.e., associated with mediating removal of ubiquitin moieties from a ubiquitylated target substrate protein). Preferably, the de-ubiquitylating agent, e.g., DUB, hydrolyze the isopeptide bond between the ubiquitin moiety

Exemplary DUBs are known in the art, and are within the scope of the present invention. DUBs useful in the invention include naturally occurring alleles and man-made variants of a DUB. In one embodiment, the de-ubiquitylation agent comprises an amino acid sequence or a nucleic acid sequence of a sequence corresponding to an accession number in the Genbank data base or ENSEMBL data base (a joint project of the European Molecular Biology Laboratories and the Sanger Institute) listed in the Table 4 below. The accession numbers from the Genbank data base and the ENSEMBL database can be obtained from the websites supported by these organizations.

TABLE 4

| | |
|---|---|
| nucleic acid | amino acid |

Genbank Acession Nos.

| | |
|---|---|
| XM_086378 | XP_086378 |
| XM_088736 | XP_088736 |
| NM_024292 | NP_077268 |
| M10939 | AAA36788 |
| NM_007278 | NP_009209 |
| XM_086494 | XP_086494 |
| NM_007285 | NP_009216 |
| NM_014235 | NP_055050 |
| BC012472 | AAH12472 |
| AF251700 | AAL99389 |
| XM_063384 | XP_063384 |
| XM_064899 | XP_064899 |
| BC008450 | AAH08450 |
| XM_030786 | XP_030786 |
| BC019910 | AAH19910 |
| BC014367 | AAH14367 |
| NM_032514 | NP_115903 |
| NM_001997 | NP_001988 |
| XM_087907 | XP_087907 |
| AK026593 | BAB15505 |
| XM_092407 | XP_092407 |
| XM_113737 | XP_113737 |
| AF348700 | AAK31162 |
| AF077046 | AAD27779 |
| NM_003333 | NP_003324 |
| XM_089415 | XP_089415 |
| NM_006156 | NP_006147 |
| XM_114058 | XP_114058 |
| XM_168354 | XP_168354 |
| NM_004707 | NP_004698 |
| NM_007106 | NP_009037 |
| NM_007108 | NP_009039 |
| NM_032568 | NP_115957 |
| NM_002954 | NP_002945 |
| NM_003352 | NP_003343 |
| NM_005101 | NP_005092 |
| NM_006936 | NP_008867 |
| XM_009805 | XP_009805 |
| XM_115124 | XP_115124 |
| BC011033 | AAH11033 |
| NM_024571 | NP_078847 |
| XM_093349 | XP_093349 |
| XM_091851 | XP_091851 |
| XM_166749 | XP_166749 |
| NM_022818 | NP_073729 |
| XM_058745 | XP_058745 |
| XM_066029 | XP_066029 |
| NM_006398 | NP_006389 |
| NM_003363 | NP_003354 |
| NM_006313 | NP_006304 |
| AF383173 | AAL78315 |
| AF130096 | AAG35521 |
| AB029020 | BAA83049 |
| BC003130 | AAH03130 |
| XM_113421 | XP_113421 |
| NM_004654 | NP_004645 |
| XM_166244 | XP_166244 |
| XM_070195 | XP_070195 |
| XM_167111 | XP_167111 |
| NM_003470 | NP_003461 |
| XM_065679 | XP_065679 |
| XM_093206 | XP_093206 |
| AF353989 | AAK49524 |
| AF217979 | AAG17222 |
| NM_014871 | NP_055686 |
| AK001647 | BAA91807 |
| BC016146 | AAH16146 |
| NM_020903 | NP_065954 |
| BC013737 | AAH13737 |
| AB046814 | BAB13420 |
| NM_031907 | NP_114113 |
| NM_006590 | NP_006581 |
| XM_032614 | XP_032614 |
| BC026072 | AAH26072 |
| XM_033922 | XP_033922 |
| BC000263 | AAH00263 |
| AK022574 | BAB14107 |
| AF035620 | AAC24200 |
| XM_038934 | XP_038934 |
| NM_017414 | NP_059110 |
| XM_165948 | XP_165948 |
| XM_033017 | XP_033017 |
| NM_022832 | NP_073743 |
| XM_113381 | XP_113381 |
| NM_015247 | NP_056062 |
| Y13619 | CAA73941 |
| XM_005624 | XP_005624 |
| XM_165946 | XP_165946 |
| XM_003288 | XP_003288 |
| AK022864 | BAB14279 |
| XM_042698 | XP_042698 |
| AB040886 | BAA95977 |
| XM_115909 | XP_115909 |
| AF077040 | AAD27773 |
| AK022759 | BAB14232 |
| NM_004652 | NP_004643 |
| NM_032147 | NP_115523 |
| NM_006044 | NP_006035 |
| NM_020886 | NP_065937 |
| XM_093148 | XP_093148 |
| AB067478 | BAB67784 |
| XM_036729 | XP_036729 |
| XM_030130 | XP_030130 |
| XM_050754 | XP_050754 |
| NM_032582 | NP_115971 |
| NM_021906 | NP_068706 |
| BC009452 | AAH09452 |
| AB037793 | BAA92610 |
| XM_027038 | XP_027038 |
| XM_034123 | XP_034123 |
| XM_007903 | XP_007903 |
| AK024318 | BAB14881 |
| AK027820 | BAB55392 |
| AB020656 | BAA74872 |
| NM_015017 | NP_055832 |
| XM_166526 | XP_166526 |
| XM_093964 | XP_093964 |
| XM_027791 | XP_027791 |
| NM_006768 | NP_006759 |
| NM_006676 | NP_006667 |
| XM_027039 | XP_027039 |
| XM_165973 | XP_165973 |
| XM_068007 | XP_068007 |
| AK055188 | BAB70869 |
| NM_004651 | NP_004642 |
| XM_051386 | XP_051386 |
| AF017306 | AAC27356 |
| BC018113 | AAH18113 |
| XM_058840 | XP_058840 |
| NM_025090 | NP_079366 |
| XM_028405 | XP_028405 |
| AK027362 | BAB55063 |
| XM_046769 | XP_046769 |
| NM_032236 | NP_115612 |
| NM_032663 | NP_116052 |
| AF000986 | AAC51833 |
| NM_016572 | NP_057656 |
| XM_114325 | XP_114325 |
| NM_032557 | NP_115946 |
| NM_005151 | NP_005142 |
| XM_068006 | XP_068006 |
| NM_006537 | NP_006528 |
| BC022094 | AAH22094 |
| AF233442 | AAF61308 |
| AB033029 | BAA86517 |
| D80012 | BAA11507 |
| AK001671 | BAA91825 |
| AF161450 | AAF29010 |
| XM_093962 | XP_093962 |
| NM_012475 | NP_036607 |
| XM_047413 | XP_047413 |
| AF153604 | AAD41086 |
| NM_006447 | NP_006438 |
| NM_005154 | NP_005145 |
| BC000350 | AAH00350 |
| AF174499 | AAF36540 |
| BC011576 | AAH11576 |
| AF155116 | AAD42882 |
| AF113219 | AAG39290 |
| AK026930 | BAB15591 |
| XM_033651 | XP_033651 |
| BC016663 | AAH16663 |
| XM_167944 | XP_167944 |
| BC015930 | AAH15930 |
| AF079564 | AAC28392 |
| NM_003481 | NP_003472 |
| NM_013396 | NP_037528 |
| AB040948 | BAA96039 |
| AB011142 | BAA25496 |
| XM_049683 | XP_049683 |
| BC025317 | AAH25317 |
| AF161542 | AAF29029 |
| AJ012755 | CAA10171 |
| NM_003940 | NP_003931 |
| XM_034147 | XP_034147 |
| AK057992 | BAB71627 |
| AY008763 | AAG33252 |
| AF335474 | AAK69630 |
| NM_015670 | NP_056485 |
| AB051494 | BAB21798 |
| XM_114357 | XP_114357 |
| BC028583 | AAH28583 |
| AB018340 | BAA34517 |
| XM_011455 | XP_011455 |
| NM_014554 | NP_055369 |
| BC008589 | AAH08589 |
| BC030705 | AAH30705 |
| AF308450 | AAL06294 |
| XM_084114 | XP_084114 |
| XM_058689 | XP_058689 |
| XM_113930 | XP_113930 |
| AB037752 | BAA92569 |
| AK027599 | BAB55222 |
| NM_021627 | NP_067640 |
| NM_020654 | NP_065705 |
| AB051514 | BAB21818 |
| AF199458 | AAL25651 |
| AF217504 | AAG09703 |
| NM_015571 | NP_056386 |
| XM_034262 | XP_034262 |

EMSEMBL Accession No.s

| | |
|---|---|
| ENST00000264281 | ENSP00000264281 |
| ENST00000281393 | ENSP00000281393 |
| ENST00000279003 | ENSP00000279003 |
| ENST00000296943 | ENSP00000296943 |
| ENST00000253105 | ENSP00000253105 |
| ENST00000241470 | ENSP00000241470 |
| ENST00000262306 | ENSP00000262306 |
| ENST00000285285 | ENSP00000285285 |
| ENST00000299678 | ENSP00000299678 |
| ENST00000250495 | ENSP00000250495 |
| ENST00000300630 | ENSP00000300630 |
| ENST00000294574 | ENSP00000294574 |
| ENST00000275108 | ENSP00000275108 |
| ENST00000259937 | ENSP00000259937 |
| ENST00000218299 | ENSP00000218299 |
| ENST00000286669 | ENSP00000286669 |
| ENST00000247526 | ENSP00000247526 |
| ENST00000291615 | ENSP00000291615 |
| ENST00000294270 | ENSP00000294270 |
| ENST00000274459 | ENSP00000274459 |
| ENST00000218154 | ENSP00000218154 |
| ENST00000258728 | ENSP00000258728 |
| ENST00000229699 | ENSP00000229699 |
| ENST00000003302 | ENSP00000003302 |
| ENST00000209500 | ENSP00000209500 |
| ENST00000215794 | ENSP00000215794 |
| ENST00000218348 | ENSP00000218348 |
| ENST00000219473 | ENSP00000219473 |
| ENST00000219689 | ENSP00000219689 |
| ENST00000226440 | ENSP00000226440 |
| ENST00000229268 | ENSP00000229268 |
| ENST00000232487 | ENSP00000232487 |
| ENST00000250066 | ENSP00000250066 |
| ENST00000251722 | ENSP00000251722 |
| ENST00000251784 | ENSP00000251784 |
| ENST00000252403 | ENSP00000252403 |
| ENST00000254181 | ENSP00000254181 |
| ENST00000257011 | ENSP00000257011 |
| ENST00000257548 | ENSP00000257548 |
| ENST00000258123 | ENSP00000258123 |
| ENST00000258399 | ENSP00000258399 |
| ENST00000258499 | ENSP00000258499 |
| ENST00000259103 | ENSP00000259103 |
| ENST00000259404 | ENSP00000259404 |
| ENST00000260187 | ENSP00000260187 |
| ENST00000260188 | ENSP00000260188 |
| ENST00000260419 | ENSP00000260419 |
| ENST00000261497 | ENSP00000261497 |
| ENST00000261601 | ENSP00000261601 |
| ENST00000261737 | ENSP00000261737 |
| ENST00000261843 | ENSP00000261843 |
| ENST00000262773 | ENSP00000262773 |
| ENST00000263184 | ENSP00000263184 |
| ENST00000263311 | ENSP00000263311 |
| ENST00000263858 | ENSP00000263858 |
| ENST00000263966 | ENSP00000263966 |
| ENST00000264208 | ENSP00000264208 |
| ENST00000265452 | ENSP00000265452 |
| ENST00000265560 | ENSP00000265560 |
| ENST00000265831 | ENSP00000265831 |
| ENST00000268049 | ENSP00000268049 |
| ENST00000269134 | ENSP00000269134 |
| ENST00000271487 | ENSP00000271487 |
| ENST00000276019 | ENSP00000276019 |
| ENST00000276060 | ENSP00000276060 |
| ENST00000280377 | ENSP00000280377 |
| ENST00000280395 | ENSP00000280395 |
| ENST00000282088 | ENSP00000282088 |
| ENST00000282344 | ENSP00000282344 |
| ENST00000284174 | ENSP00000284174 |
| ENST00000285199 | ENSP00000285199 |
| ENST00000285679 | ENSP00000285679 |
| ENST00000285681 | ENSP00000285681 |
| ENST00000286782 | ENSP00000286782 |
| ENST00000289865 | ENSP00000289865 |
| ENST00000292729 | ENSP00000292729 |
| ENST00000294383 | ENSP00000294383 |
| ENST00000294617 | ENSP00000294617 |
| ENST00000295040 | ENSP00000295040 |
| ENST00000295041 | ENSP00000295041 |
| ENST00000296572 | ENSP00000296572 |
| ENST00000297228 | ENSP00000297228 |
| ENST00000297229 | ENSP00000297229 |
| ENST00000298462 | ENSP00000298462 |
| ENST00000299574 | ENSP00000299574 |
| ENST00000300924 | ENSP00000300924 |

In one embodiment of particular interest, the DUB is USP-25, which is described in U.S. Patent Application Publications US 2003/0036107 and US 2003/0092605, each of which is incorporated herein by reference in its entirety.

The skilled artisan will appreciate that many different DUB proteins and isozymes are known in the field and may be used in the present invention, provided that the DUB has the de-ubiquitylating activity. Also specifically included within the term “DUB” are variants of DUB, which can be made as described herein.

De-ubiquitylating agent variants contemplated by the invention include de-ubiquitylating agent variants that retain a characteristic of a native ubiquitin de-ubiquitylating agent in being capable of facilitating removal of a ubiquitin moiety from a ubiquitylated target substrate protein. Such de-ubiquitylating agent variants generally have an overall amino acid sequence identity of preferably greater than about 75%, more preferably greater than about 80%, even more preferably greater than about 85% and most preferably greater than 90% of an amino acid sequence of a de-ubiquitylating agent provided above. In some embodiments the sequence identity will be as high as about 93% to 95% or 98%. Variants of de-ubiquitylating agents and other components of the assays of the invention are described below in more detail.

Target Proteins

By “target protein” or “substrate protein” or “ubiquitin ligase substrate” herein is meant a protein other than a ubiquitin moiety to which a ubiquitin moiety is bound or attached through the activity of a ubiquitin agent or by the process of ubiquitylation, and/or a protein other than a ubiquitin moiety from which a ubiquitin moiety is removed, e.g., through action of a de-ubiquitylating agent. In general the target protein is a mammalian protein, and more preferably a human protein. That is, as used herein, “substrate molecule” or “target substrate” and grammatical equivalents thereof means a molecule, preferably a protein, to which a ubiquitin moiety is bound or attached through the activity of a ubiquitin agent or by the process of ubiquitylation.

As used herein with reference to the activity of ubiquitin agents, “attachment” refers to the transfer, binding, ligation, and/or ubiquitylation of a mono- or poly-ubiquitin ubiquitin moiety to a substrate molecule. Thus, “ubiquitylation” and grammatical equivalents thereof means the attachment, or transfer, binding, and/or ligation of ubiquitin moiety to a substrate molecule; and “ubiquitylation reaction” and grammatical equivalents thereof refer to the combining of components under conditions that permit ubiquitylation (i.e., the attachment or transfer, binding, and/or ligation of ubiquitin moiety to a substrate molecule).

The host cell substrate protein of interest is one that has activity against a retroviral infection of the cell. The substrate protein can be one that renders the host cell completely or partially non-permissive to retroviral infection, particularly to retroviral replication. Ubiquitylation of the host cell protein results in a decrease in availability of the protein to exhibit its antiviral action, e.g., due to proteosome-mediated degradation.

In one embodiment, the substrate protein is a host cell cytosine deaminase, particularly CEM15. CEM15 is also known as APOBEC3G, apolipoprotein B mRNA editing enzyme, and catalytic polypeptide-like 3G. CEM15 is a host cell cytidine deaminse that induces hypermutation in newly synthesized HIV-1 DNA. CEM15 acts as a deaminase to convert C's to U's in the DNA minus strand produced from retroviral RNA during retroviral replication. When the plus strand of the CEM15-modified virus strand is produced, the plus stand contains G-to-A hypermutation. An exemplary nucleotide and amino acid sequence of human CEM15 is found in GenBank Accession No. NM_{—}021822. Proteins related to CEM15 have been described. For a review, see, e.g., Wedekind et al., Trends Genet. 19(4):207-16 (2003).

In another embodiment, the substrate protein is CD4. CD4 is a T-cell antigen, which is also known as T4/leu3. expressed not only in T lymphocytes, but also in B cells, macrophages, and granulocytes. It is also expressed in a developmentally regulated manner in specific regions of the brain. CD4, which binds to relatively invariant sites on class II major histocompatibility complex (MHC) molecules outside the peptide-binding groove, which interacts with the T-cell receptor (TCR), enhances T-cell sensitivity to antigen. CD4, acting together with a chemokine receptor such as CCR5 ro CXCR5, especially CCR5, mediates HIV binding and internalization in the initial stages of infection. An exemplary nucleotide and amino acid sequence of human CD4 is found in GenBank Accession No. P01730.

Sequences encoding host cell substrate proteins may also be used to make variants thereof that are suitable for use in the methods and compositions of the present invention. Such variants suitable for use in the methods and compositions of the present invention may be made as described herein. Substrate protein variants contemplated by the invention include substrate protein variants that retain a characteristic of a native substrate protein variant in being capable of ubiquitylation by attachment of an ubiquitin moiety as facilitated by one or more ubiquitylation cascade proteins. Where the target protein is ubiquitylated, the variant is one that retains a characteristic of the native ubiquitylated protein in being capable of modification by removal of a ubiquitin moiety by a de-ubiquitylating agent. Such target protein variants generally have an overall amino acid sequence identity of preferably greater than about 75%, more preferably greater than about 80%, even more preferably greater than about 85% and most preferably greater than 90% of an amino acid sequence of a native target protein, e.g, CEM15 or CD4. In some embodiments the sequence identity will be as high as about 93% to 95% or 98%. Variants of substrate proteins and other components of the assays of the invention are described below in more detail.

As with other proteins indicated for use in the invention, guidance for production of variants of substrate proteins can be accomplished by examining the motifs of the protein as well as the sequences conserved with related proteins, and making amino acid substitutions, deletions, and/or insertions accordingly.

For an exemplary discussion of CEM15 variants, see, e.g, Shindo et al. J. Biol. Chem. 2003 278:44412-6, which describes extensive mutagenesis analysis on two cytidine deaminase motifs in CEM15 and the effect upon the function of the protein in its affect upon virion infectivity as well as the DNA editing activity. For example, point mutations in the C-terminal active site such as E259Q and C291A almost completely abrogated the antiviral function, while those in the N-terminal active site such as E67Q and C100A retained this activity to a lesser extent as compared with that of the wild type (the nomenclature used here provides the native residue in single letter code, the residue number within CEM15, and the residue substituted at that positioning single letter code). The DNA editing activities of E67Q and E259Q mutants were both retained, but impaired to the same extent. This indicates that the enzymatic activity of CEM15 is essential, but not a sole determinant of the antiviral activity of the protein.

Retroviruses and Retroviral Proteins

The screening methods of the invention can be used to identify agents that modulate ubiquitylation of a host cell substrate protein (e.g., CEM15) in a cell infected with any of a variety of retroviruses or in a cell containing (e.g., by virtue of expression of an exogenous polynucleotide or introduction of a polypeptide into the cell) a retroviral protein that modulates or is suspecting of modulating ubiquitylation of a host cell protein, where the host cell protein confers on the cell an antiviral activity. In general, such retroviral proteins enhance ubiquitylation of such antiviral host cell proteins so as to enhance the permissiveness of the host cell to retroviral replication.

In one embodiment discussed in more detail below, the assay is conducted using a host cell that is infected with a retrovirus of interest. Retroviruses of interest include lentiviruses (e.g., HIV (including subtypes of HIV such as HIV-1 and HIV-2), simian immunodeficiency virus (SIV), equine infectious anemia virus (EAV), caprine arthritis encephalitis virus (CAEV), visna/maedi virus); alpharetroviruses (e.g., avian leucosis virus (ALV); Rous sarcoma virus (RSV)); betaretroviruses (e.g., mouse mammary tumor virus (MMTV), Mason-Pfizer monkey virus (MPMV), Jaagsiekte sheep retrovirus (JSRV)); gammaretroviruses (e.g., murine leukemia viruses (MuLv); feline leukemia viruses (FeLv), gibbon ape leukemia virus (GALV), reticuloendotheliosis virs (RevT)); deltaretroviruses (e.g., human T-lymphotropic viruses (HTLV, including HTLV-1 and HTLV-2), bovine leukemia virus (BLV), simian T-lymphotropic virus (STLV, including STLV-1, -2, and -3)); epsilonretroviruses (e.g., walleye dermal sarcoma virus, walleye epidermal hyperplasia virus 1); and spumavirus (e.g., human foamy virus (HFV), and the like.

In another embodiment, the assay is conducted using a retroviral protein that is a viral ubiquitylation modulator protein, where the assay is conducted either as a cell-free or cell-based assay. A “viral ubiquitylation modulator protein” refers to a viral protein that modulates, usually promotes, ubiquitylation of a host cell substrate protein, particular a host cell substrate protein that has an antiviral activity. In this embodiment, isolated retroviral protein or nucleic acid encoding the isolated retroviral protein are used.

Vif

Vif is a retroviral ubiquitylation modulator protein of particular interest in the screening methods of the invention. Vif is a highly basic protein of HIV which is synthesized at relatively high levels late in the viral replication cycle. In general, native Vif is about 23 KDa and about 192 amino acid residues in length, and is phosphorylated. Vif is highly conserved in HIV-1 and other lentiviruses including simian immunodeficiency virus (SIV) an feline immunodeficiency virus (FIV), and is required for productive infection in vivo. Vif is not necessarily required for replication of HIV-1 in all cells, but is required for HIV-1 replication in primary T lymphocytes and monocytes/macrophages. The differences in requirement for of Vif for replication has led to categorization of primary cells or cell lines as either non-permissive (H9, HYT78, A3.0, primary CD4+ T-cells), semi-permissive (CEM-ss, monocyte derived macrophages (MDM)) or permissive (HeLa, 293T, Cos-7, SupT1) for replication of Vif-defective viruses (see, e.g., Sova et al. J Viol 67:6322-6366 (1993); Zhang et al. J virol 74:8252-8261 (2000)). For a review regarding Vif, see, e.g., Lake et al. J Clin Virol. 26(2):143-52. (2003). Vif of HIV-1 is of particular interest in the present invention.

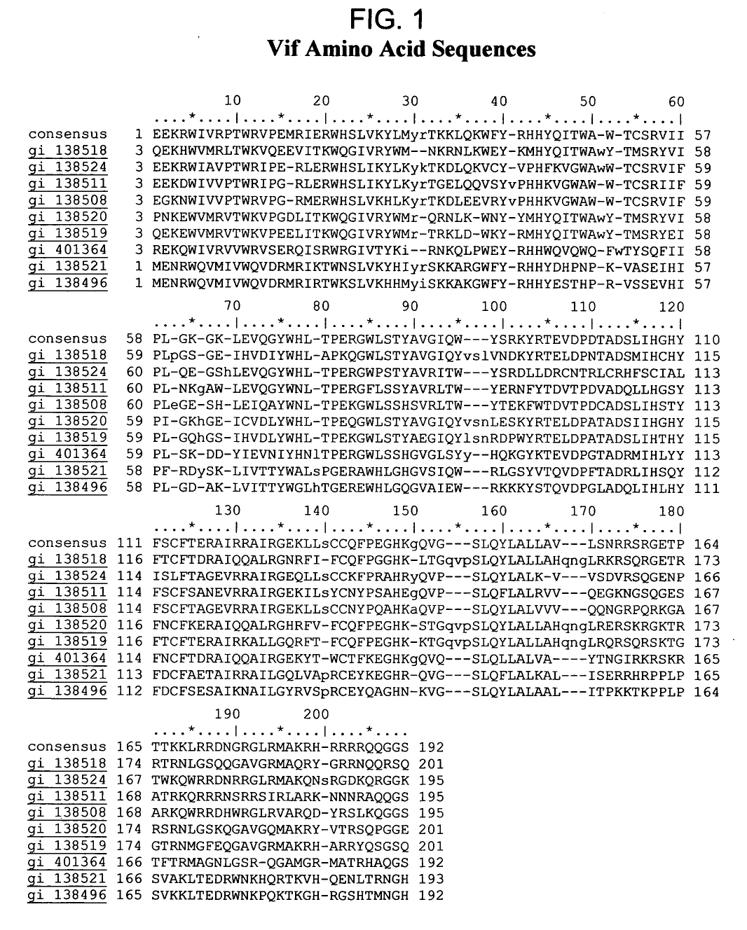

Nucleotide and amino acid sequences for Vif are known in the art. A consensus sequence for HIV-1 Vif is provided in Pfam accession no. pfam00559. This consensus sequence, as well as exemplary Vif amino acid sequences, are provided in FIG. 1. Guidance for amino acid changes can be derived from alignments such as those in FIG. 1, as well as alignments of other Vif amino acid sequences of other strains of HIV-1 as well as other retroviruses.

The invention contemplates use of Vif variants for use in the methods and compositions of the present invention which variants may be made as described herein. Vif variants contemplated by the invention include Vif variants that retain a characteristic of a native Vif in being capable of modulating the host cell ubiquitylation cascade so as to effect an increase in ubiquitylation of a host cell substrate protein, particularly CEM15. Such Vif variants generally have an overall amino acid sequence identity of preferably greater than about 75%, more preferably greater than about 80%, even more preferably greater than about 85% and most preferably greater than 90% of an amino acid sequence of a native Vif protein. In some embodiments the sequence identity will be as high as about 93% to 95% or 98%. Vif variants and other components of the assays of the invention are described below in more detail.

Vpu

Vpu is a retroviral ubiquitylation modulator protein of particular interest in the screening methods of the invention. Vpu protein stimulates virus production by enhancing the release of viral particles from infected cells. The Vpu protein binds specifically to CD4. Vpu also targets the CD4 receptor for proteasomal degradation by rectruing the SCF^{βTrCp} ubiquitin E3 ligase complex to the cotyplasmic tail of CD4.

In general, native Vpu is about 81 amino acid residues in length, and is present in HIV (especially HIV-1) and SIV. Vpu is a phosphorylated oligomeric type 1 integral membrane proteins. Vpu performs two main functions during viral life cycle—it enhances release of virions from infected cells, and it mediates the selective degradation of the CD4 receptor in the endoplasmic reticulum. Vpu protein binds specifically to CD4. Vpu also targets the CD4 receptor for proteasomal degradation by recruiting the SCF^{βTrCp} ubiquitin E3 ligase complex to the cytoplasmic tail of CD4. Vpu of HIV, especially, HIV-1 is of particular interest in the present invention.

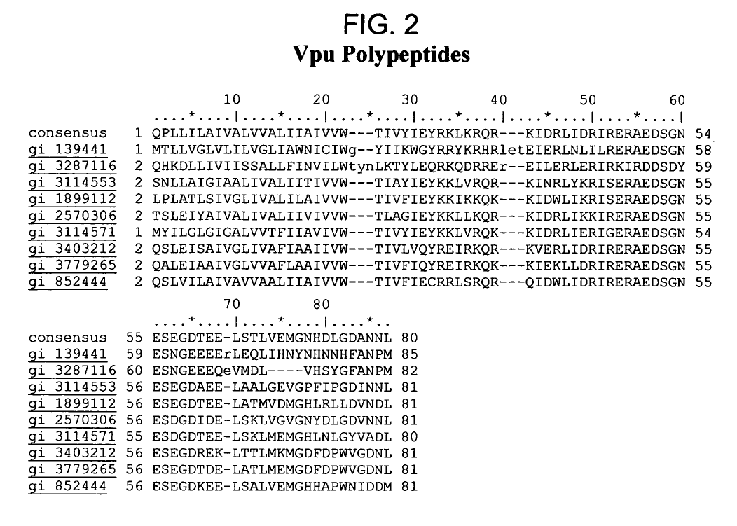

Nucleotide and amino acid sequences for Vpu are known in the art. A consensus sequence for HIV-1 Vpu is provided in Pfam accession no. pfam00558.8. This consensus sequence, as well as exemplary Vpu amino acid sequences, are provided in FIG. 2. Guidance for amino acid changes can be derived from alignments such as those in FIG. 2, as well as alignments of other Vpu amino acid sequences of other strains of HIV-1 as well as other retroviruses.

The invention contemplates use of Vpu variants for use in the methods and compositions of the present invention which variants may be made as described herein. Vpu variants contemplated by the invention include Vpu variants that retain a characteristic of a native Vpu in being capable of modulating the host cell ubiquitylation cascade so as to effect an increase in ubiquitylation of a host cell substrate protein, particularly CD4. Such Vpu variants generally have an overall amino acid sequence identity of preferably greater than about 75%, more preferably greater than about 80%, even more preferably greater than about 85% and most preferably greater than 90% of an amino acid sequence of a native Vpu protein. In some embodiments the sequence identity will be as high as about 93% to 95% or 98%. Vpu variants and other components of the assays of the invention are described below in more detail.

Variant Polypeptides Differing in Amino Acid Sequence and Fragments

As noted above, the assays of the invention described herein can be conducted with variants of the various proteins involved in the ubiquitylation cascade, including variants of ubiquitin, E1, E2, E3, de-ubiquitylating enzymes (DUBs), substrate proteins (e.g., CEM15), and retroviral ubiquitylation modulator proteins (e.g., Vif, Vpu). These variants generally fall into one or more of three classes: substitutional, insertional or deletional variants. Variants are generally described as having a sequence similarity (e.g., sequence identity) relative to that of a “reference” sequence, e.g., the sequence of the naturally-occurring protein. It will also be readily appreciated that proteins that share amino acid sequence similarity are encoded by nucleic acids that share nucleotide sequence similarity.

As is known in the art, a number of different programs can be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25:3389-3402. Gapped BLAST uses BLOSUM-62 substitution scores; threshold T parameter set to 9; the two-hit method to trigger ungapped extensions; charges gap lengths of k a cost of 10+k; Xu set to 16, and Xg set to 40 for database search stage and to 67 for the output stage of the algorithms. Gapped alignments are triggered by a score corresponding to ˜22 bits.

A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The alignment may include the introduction of gaps in the sequences to be aligned. In addition, for sequences which contain either more or fewer amino acids than the reference amino acid sequence, it is understood that in one embodiment, the percentage of sequence identity will be determined based on the number of identical amino acids in relation to the total number of amino acids. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Variants of interest can ordinarily be prepared by site specific mutagenesis of nucleotides in the DNA encoding a protein of the present compositions, using cassette or PCR mutagenesis or other techniques well known in the art, to produce DNA encoding the variant, and thereafter expressing the DNA in recombinant cell culture as outlined above. However, variant protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed variants screened for the optimal desired activity. Techniques for making substitution mutations at predetermined sites in DNA having a known sequence are well known, for example, M13 primer mutagenesis and PCR mutagenesis. Rapid production of many variants may be done using techniques such as the method of gene shuffling, whereby fragments of similar variants of a nucleotide sequence are allowed to recombine to produce new variant combinations. Examples of such techniques are found in U.S. Pat. Nos. 5,605,703; 5,811,238; 5,873,458; 5,830,696; 5,939,250; 5,763,239; 5,965,408; and 5,945,325, each of which is incorporated by reference herein in its entirety. Screening of the mutants is performed using the activity assays of the present invention.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the protein are desired, substitutions of an original residue are generally made in accordance with exemplary substitutions listed below.

Table of Exemplary Amino Acid Substitutions

| | |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in the above list. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

In one embodiment, the variants typically exhibit the same qualitative biological activity and will elicit the same immune response as the naturally-occurring analogue, although variants also are selected to modify the characteristics of the proteins as needed. Alternatively, the variant may be designed such that the biological activity of the protein is altered. For example, glycosylation sites may be altered or removed.

It will be appreciated that the nucleotide sequences of protein variants can be readily determined, for example based upon the amino acid sequence of the variant and the knowledge of the genetic code. Due to the degeneracy of the genetic code, a nucleotide sequence encoding a protein variant may exhibit a lower sequence identity with the corresponding native nucleotide sequence than the amino acid sequence identity between the variant protein and the native protein. For example, nucleotide sequences share as little as about 66% (i.e., about ⅔) nucleotide sequence identity can encode the same amino acid sequence due to the degeneracy of the genetic code. Thus, nucleic acid encoding a protein variant can have at least 30%, 40%, 50%, 60%, 70%, 80%, 85%, 90%, or 95% sequence identity with a reference nucleic acid, for example, the corresponding nucleic acid encoding the native protein (i.e., the protein prior to modification) from which a variant protein sequence is derived.

The invention also contemplates use of ubiquitin proteins, substrate proteins, and retroviral ubiquitylation modulator proteins which are shorter or longer than the corresponding naturally occurring amino acid sequence. That is, portions or fragments of the proteins described herein can be used in the assays of the invention. The fragments of use in the invention retain a biological activity of the protein from which it was derived or with which it shares amino acid sequence identity. For example, a ubiquitin fragment useful in the invention is one that can be transferred (or removed from) a substrate protein by the corresponding ubiquitin agents. Similarly, a fragment of a ubiquitin activating agent (e.g., a fragment of E1) of interest is one that retains activity in being modified by a ubiquitin moiety and activating a ubiquitin conjugating agent. A fragment of a ubiquitin conjugating agent (e.g., a fragment of E2) of interest is one that retains activity in interacting with an E3 to facilitate transfer of a ubiquitin moiety to a substrate protein. A ubiquitin ligating agent fragment retains activity in interacting with a target protein and an activated E2 to facilitate transfer of a ubiquitin moiety to the target protein. A target protein fragment of interest is one that can be modified by attachment of and/or removal of ubiquitin moieties by the relevant components of the ubiquitin cascade. A retroviral ubiquitylation modulator fragment of interest is one that retains activity in modulating ubiquitylation, e.g., a Vif fragment that retains activity in enhancing ubiquitylation of CEM15; a Vpu fragment that retains activity in enhancing ubiquitylation of CD4.

Production of Polypeptides

Ubiquitylation cascade agents (e.g., ubiquitin moieties and other ubiquitin agents), and target substrate proteins, and viral proteins that modulate ubiquitylation of target substrate proteins for use in the methods and compositions of the present invention can be produced according to methods known in the art. In addition, probe or degenerate polymerase chain reaction (PCR) primer sequences may be used to find other related or variant ubiquitin moieties, ubiquitin agents, and target proteins from humans or other organisms. As will be appreciated by those in the art, particularly useful probe and/or PCR primer sequences include the unique areas of a nucleic acid sequence. As is generally known in the art, PCR primers are generally from about 15 to about 35 nucleotides in length, with from about 20 to about 30 being usual, and may contain inosine as needed. The conditions for the PCR reaction are well known in the art. It is therefore also understood that provided along with the sequences in the sequences cited herein are portions of those sequences, wherein unique portions of 15 nucleotides or more are particularly of interest. The skilled artisan can routinely synthesize or cut a nucleotide sequence to the desired length.

Once isolated from its natural source, e.g., contained within a plasmid or other vector or excised therefrom as a linear nucleic acid segment, the recombinant nucleic acid can be further-used as a probe to identify and isolate other nucleic acids. It can also be used as a “precursor” nucleic acid to make modified or variant nucleic acids and proteins.

In one embodiment, the nucleic acids of the invention are part of an expression vector. Using the nucleic acids of the present invention which encode a protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. As another example, operably linked refers to DNA sequences linked so as to be contiguous, and, in the case of a secretory leader, contiguous and in reading frame. However, enhancers do not have to be contiguous. Linking is accomplished by ligation at convenient restriction sites. If such sites do not exist, the synthetic oligonucleotide adapters or linkers are used in accordance with conventional practice. The transcriptional and translational regulatory nucleic acid will generally be appropriate to the host cell used to express the protein; for example, transcriptional and translational regulatory nucleic acid sequences from Bacillus can be used to express the protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In one embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in one embodiment, the expression vector contains a selectable marker gene to allow the selection of transformed host cells. Selection genes are well known in the art and will vary with the host cell used.

An exemplary expression vector system is a retroviral vector system such as is generally described in PCT/US97/01019 and PCT/US97/01048, both of which are hereby expressly incorporated by reference. Constructs also are described in U.S. Pat. No. 6,153,380, which is expressly incorporated herein by reference.

Proteins of the present invention are produced by culturing a host cell transformed with an expression vector containing nucleic acid encoding the protein, under the appropriate conditions to induce or cause expression of the protein. The conditions appropriate for protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction.

Appropriate host cells include yeast, bacteria, archaebacteria, fungi, and insect and animal cells, including mammalian cells. Of particular interest are Drosophila melanogaster cells, Pichia pastoris and P. methanolica, Saccharomyces cerevisiae and other yeasts, E. coli, Bacillus subtilis, SF9 cells, SF21 cells, C129 cells, Saos-2 cells, Hi-cells, 293 cells, Neurospora, BHK, CHO, COS, and HeLa cells. Of greatest interest are A549, HeLa, HUVEC, Jurkat, BJAB, CHMC, and cell lines derived from T cells or macrophage.

In a one embodiment, the proteins are expressed in mammalian cells, especially human cells. Mammalian expression systems are also known in the art, and include retroviral systems. A mammalian promoter (i.e., a promoter functional in a mammalian cell) is any DNA sequence capable of binding mammalian RNA polymerase and initiating the downstream (3′) transcription of a coding sequence for a protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

Where the host cell is a bacterial cell, a suitable bacterial promoter is any nucleic acid sequence capable of binding bacterial RNA polymerase and initiating the downstream (3′) transcription of the coding sequence of a protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

The protein may also be made as a fusion protein, using techniques well known in the art. Thus, for example, the protein may be made fusion nucleic acid encoding the peptide or may be linked to other nucleic acid for expression purposes. Similarly, proteins of the invention can be linked to tags that are protein labels, such as green fluorescent protein (GFP), red fluorescent protein (RFP), blue fluorescent protein (BFP), yellow fluorescent protein (YFP), luciferase, etc. The fusions may include other constructs as well, including separation sites such as 2a site and internal ribosomal entry sites IRES, which are particularly useful in the construct as IRES-label to provide a method of tracking infected cells.

Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others. In one embodiment, proteins are produced in insect cells. Expression vectors for the transformation of insect cells, and in particular, baculovirus-based expression vectors, are well known in the art. In another embodiment, proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii P. methanolica and P. pastoris, Schizosaccharomyces pombe, and Yarrowia lipolytica. Promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TW1, and ALG7, which confers resistance to tunicamycin; the neomycin phosphotransferase gene, which confers resistance to G4 18; and the CUP 1 gene, which allows yeast to grow in the presence of copper ions.

Proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the ubiquitin protein may be purified using a standard anti-ubiquitin antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the protein. In some instances no purification will be necessary.

Covalently Modified Proteins, Including Detectably Labeled Ubiquitin Agents

In one embodiment, covalent modifications of polypeptides are included within the scope of this invention. Such covalent modifications generally find use in in vitro assays as described in more detail in U.S. Ser. No. 09/800,770, filed Mar. 6, 2001, which is expressly incorporated herein by reference.

Tagged Polypeptides

Agents, particularly ubiquitylation cascade agents (e.g., ubiquitylation cascade proteins), host cell ubiquitylation substrate proteins, and retroviral ubiquitylation modulator proteins (e.g., Vif, Vpu) can be modified so that they comprise a tag. By “tag” is meant an attached molecule or molecules useful for the identification or isolation of the attached molecule(s), which can be substrate binding molecules. For example, a tag can be an attachment tag or a label tag. Components having a tag are referred to as “tag-X”, wherein X is the component. For example, a ubiquitin moiety comprising a tag is referred to herein as “tag-ubiquitin moiety”. Preferably, the tag is covalently bound to the attached component.

When more than one component of a combination has a tag, the tags will be numbered for identification, for example “tag1-ubiquitin moiety”. Components may comprise more than one tag, in which case each tag will be numbered, for example “tag 1,2-ubiquitin moiety”. Exemplary tags include, but are not limited to, a label, a partner of a binding pair, and a surface substrate binding molecule (or attachment tag). As will be evident to the skilled artisan, many molecules may find use as more than one type of tag, depending upon how the tag is used. In one embodiment, the tag or label as described below is incorporated into the polypeptide as a fusion protein.

As will be appreciated by those in the art, tag-components of the invention can be made in various ways, depending largely upon the form of the tag. Components of the invention and tags are preferably attached by a covalent bond. Examples of tags are described below.

Exemplary Tags Useful in the Invention

As noted above, “tags” can be any of a variety of labels, which can be detected either directly or indirectly. Tagged ubiquitylation cascade proteins, tagged substrate proteins, and tagged retroviral ubiquitylation modulator protein find particular use in the screening assays of the invention, described below in more detail.

By “label” or “detectable label” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is performed will depend on the label. Exemplary labels include, but are not limited to, fluorescent labels (e.g. GFP) and label enzymes.

In one embodiment, the tag is a polypeptide which is provided as a portion of a chimeric molecules comprising a first polypeptide fused to another, heterologous polypeptide or amino acid sequence. In one embodiment, such a chimeric molecule comprises a fusion of a first polypeptide (e.g., a ubiquitin moiety, ubiquitin agent, or target protein) with a tag polypeptide. The tag is generally placed at the amino- or carboxyl-terminus of the polypeptide. The tag polypeptide can be, for example, a polypeptide which provides an epitope to which an anti-tag antibody can selectively bind, a polypeptide which serves as a ligand for binding to a receptor (e.g., to facilitate immobilization of the chimeric molecule on a substrate); an enzyme label (e.g., as described further below); or a fluorescent label (e.g., as described further below). Tag polypeptides provide for, for example, detection using an antibody against the tag polypeptide, and/or a ready means of isolating or purifying the tagged polypeptide (e.g., by affinity purification using an anti-tag antibody or another type of receptor-ligand matrix that binds to the tag). In an alternative embodiment, the chimeric molecule may comprise a fusion of a polypeptide disclosed herein with an immunoglobulin or a particular region of an immunoglobulin. For a bivalent form of the chimeric molecule, such a fusion could be to the Fc region of an IgG molecule. Tags for components of the invention are defined and described in detail below.

The production of tag-polypeptides by recombinant means is within the knowledge and skill in the art. Production of FLAG-labeled proteins is well known in the art and kits for such production are commercially available (for example, from Kodak and Sigma). Methods for the production and use of FLAG-labeled proteins are found, for example, in Winston et al., Genes and Devel. 13:270-283 (1999), incorporated herein in its entirety, as well as product handbooks provided with the above-mentioned kits.

Production of proteins having His-tags by recombinant means is well known, and kits for producing such proteins are commercially available. Such a kit and its use is described in the QIAexpress Handbook from Qiagen by Joanne Crowe et al., hereby expressly incorporated by reference.

By “fluorescent label” is meant any molecule that may be detected via its inherent fluorescent properties, which include fluorescence detectable upon excitation. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 2002 Molecular Probes Handbook, 9^{th} Ed., by Richard P. Haugland, hereby expressly incorporated by reference.

Suitable fluorescent labels include, but are not limited to, green fluorescent protein (GFP; Chalfie, et al., Science 263(5148):802-805 (Feb. 11, 1994); and EGFP; Clontech-Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), -galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558), and Ptilosarcus green fluorescent proteins (PGFP) (see WO 99/49019). All of the above-cited references are expressly incorporated herein by reference.

In some instances, multiple fluorescent labels are employed. In one embodiment, at least two fluorescent labels are used which are members of a fluorescence resonance energy transfer (FRET) pair. FRET can be used to detect association/dissociation of, for example, a ubiquitin ligating agent (e.g., an E3) and a target substrate protein; a ubiquitin conjugating agent (e.g., an E2) and a target substrate protein; a ubiquitin ligating agent (e.g., an E3) and a ubiquitin conjugating agent (e.g., an E2); and the like.

FRET is phenomenon known in the art wherein excitation of one fluorescent dye is transferred to another without emission of a photon. A FRET pair consists of a donor fluorophore and an acceptor fluorophore. The fluorescence emission spectrum of the donor and the fluorescence absorption spectrum of the acceptor must overlap, and the two molecules must be in close proximity. The distance between donor and acceptor at which 50% of donors are deactivated (transfer energy to the acceptor) is defined by the Forster radius, which is typically 10-100 angstroms. Changes in the fluorescence emission spectrum comprising FRET pairs can be detected, indicating changes in the number of that are in close proximity (i.e., within 100 angstroms of each other). This will typically result from the binding or dissociation of two molecules, one of which is labeled with a FRET donor and the other of which is labeled with a FRET acceptor, wherein such binding brings the FRET pair in close proximity.

Binding of such molecules will result in an increased fluorescence emission of the acceptor and/or quenching of the fluorescence 15 emission of the donor. FRET pairs (donor/acceptor) useful in the invention include, but are not limited to, EDANS/fluorescien, IAEDANS/fluorescein, fluoresceidtetramethylrhodamhe, fluoresceidLC Red 640, fluoresceidcy 5, fluoresceidcy 5.5 and fluoresceidLC Red.

In another aspect of FRET, a fluorescent donor molecule and a nonfluorescent acceptor molecule (“quencher”) may be employed. In this application, fluorescent emission of the donor will increase when quencher is displaced from close proximity to the donor and fluorescent emission will decrease when the quencher is brought into close proximity to the donor. Useful quenchers include, but are not limited to, DABCYL, QSY 7 and QSY 33. Useful fluorescent donodquencher pairs include, but are not limited to EDANS/DABCYL, Texas RedLDABCYL, BODIPYDABCYL, Lucifer yellowDABCYL, coumarin/DABCYL and fluoresceidQSY 7 dye.

The skilled artisan will appreciate that FRET and fluorescence quenching allow for monitoring of binding of labeled molecules over time, providing continuous information regarding the time course of binding reactions. It is important to remember that ubiquitin is ligated to substrate protein by its terminal carboxyl group to a lysine residue, including lysine residues on other ubiquitin. Therefore, attachment of labels or other tags should not interfere with either of these active groups on the ubiquitin. Amino acids may be added to the sequence of protein, through means well known in the art and described herein, for the express purpose of providing a point of attachment for a label. In one embodiment, one or more amino acids are added to the sequence of a component for attaching a tag thereto, with a fluorescent label being of particular interest. In one embodiment, the amino acid to which a fluorescent label is attached is Cysteine.

By “label enzyme” is meant an enzyme which may be reacted in the presence of a label enzyme substrate which produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By “radioisotope” is meant any radioactive molecule. Suitable radioisotopes for use in the invention include, but are not limited to 14C, 3H, 32P, 33P, ³⁵S, 1251, and 131I. The use of radioisotopes as labels is well known in the art.

In addition, labels may be indirectly detected, that is, the tag is a partner of a binding pair. By “partner of a binding pair” is meant one of a first and a second moiety, wherein said first and said second moiety have a specific binding affinity for each other. Suitable binding pairs for use in the invention include, but are not limited to, antigendantibodies (for example, digoxigeninlanti-digoxigenin, dinitrophenyl (DNP)/anti-DNP, dansyl-X-anti-dansyl, Fluoresceidanti-fluorescein, Lucifer yellow/anti-lucifer yellow, and rhodamine anti-rhodamine), biotirdavid (or biotirdstreptavidin) and calmodulin binding protein (CBP)/calmodulin. Other suitable binding pairs include polypeptides such as the FLAG-peptide. (Hopp et al., BioTechnol, 6:1204-1210 (1988)); the KT3 epitope peptide (Martin et al., Science, 255:192-194 (1992)); tubulin epitope peptide (Skinner et al., J. Biol. Chem., 266: 15 163-15 166 (1991)); and the T7 gene 10 protein peptide tag (Lutz-Freyemuth et al., Proc. Natl. Acad. Sci. USA, a:6393-6397 (1990)) and the antibodies each thereto. Generally, in one embodiment, the smaller of the binding pair partners serves as the tag, as steric considerations in ubiquitin ligation may be important. As will be appreciated by those in the art, binding pair partners may be used in applications other than for labeling, such as immobilization of the protein on a substrate and other uses as described below.

As will be appreciated by those in the art, a partner of one binding pair may also be a partner of another binding pair. For example, an antigen (first moiety) may bind to a first antibody (second moiety) which may, in turn, be an antigen for a second antibody (third moiety). It will be further appreciated that such a circumstance allows indirect binding of a first moiety and a third moiety via an intermediary second moiety that is a binding pair partner to each. As will be appreciated by those in the art, a partner of a binding pair may comprise a label, as described above. It will further be appreciated that this allows for a tag to be indirectly labeled upon the binding of a binding partner comprising a label. Attaching a label to a tag which is a partner of a binding pair, as just described, is referred to herein as “indirect labeling”.

In one embodiment, the tag is surface substrate binding molecule. By “surface substrate binding molecule” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate (Field et al., Mol. Cell. Biol., 8:2159-2165 (1988)); the c-myc tag and the 8F9,3C7,6E107 G4, B7 and 9E10 antibody substrates thereto (Evan et al., Molecular and Cellular Biol, 5:3610-3616 (1985)]; and the Herpes Simplex virus glycoprotein D (gD) tag and its antibody substrate (Paborsky et al., Protein Engineering, 3(6):547-553 (1990)). In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Production of antibody-embedded substrates is well known; see Slinkin et al., Bioconj, Chem. 2:342-348 (1991); Torchilin et al., supra; Trubetskoy et al., Bioconi. Chem. 33323-327 (1992); King et al., Cancer Res. 54:6176-6185 (1994); and Wilbur et al., Bioconjugate Chem. 5:220-235 (1994) (all of which are hereby expressly incorporated by reference), and attachment of or production of proteins with antigens is described above. Calmodulin-embedded substrates are commercially available, and production of proteins with CBP is described in Simcox et al., Strategies 8:40-43 (1995), which is hereby incorporated by reference in its entirety.

Where appropriate, functionalization of labels with chemically reactive groups such as thiols, amines, carboxyls, etc. is generally known in the art. In one embodiment, the tag is functionalized to facilitate covalent attachment.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see, e.g., chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known. Methods for labeling of proteins with radioisotopes are known in the art. For example, such methods are found in Ohta et al., Molec. Cell 3:535-541 (1999), which is hereby incorporated by reference in its entirety.

The covalent attachment of the tag may be either direct or via a linker. In one embodiment, the linker is a relatively short coupling moiety, that is used to attach the molecules. A coupling moiety may be synthesized directly onto a component of the invention, ubiquitin for example, and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer.

In one embodiment, the covalent attachment is direct, that is, no linker is used. In this embodiment, the component can contain a functional group such as a carboxylic acid which is used for direct attachment to the functionalized tag. It should be understood that the component and tag may be attached in a variety of ways, including those listed above. What is important is that manner of attachment does not significantly alter the functionality of the component. For example, in tag-ubiquitin, the tag should be attached in such a manner as to allow the ubiquitin to be covalently bound to other ubiquitin to form polyubiquitin chains.

As will be appreciated by those in the art, the above description of covalent attachment of a label and ubiquitin applies equally to the attachment of virtually any two molecules of the present disclosure. In one embodiment, the tag is functionalized to facilitate covalent attachment, as is generally outlined above. Thus, a wide variety of tags are commercially available which contain functional groups, including, but not limited to, isothiocyanate groups, amino groups, haloacetyl groups, maleimides, succinimidyl esters, and sulfonyl halides, all of which may be used to covalently attach the tag to a second molecule, as is described herein. The choice of the functional group of the tag will depend on the site of attachment to either a linker, as outlined above or a component of the invention. Thus, for example, for direct linkage to a carboxylic acid group of a ubiquitin, amino modified or hydrazine modified tags will be used for coupling via carbodimide chemistry, for example using 1-ethyl-3-(3-dimethylaminopropyl)-carbodimide (EDAC) as is known in the art (see Set 9 and Set 11 of the Molecular Probes Catalog, supra; see also the Pierce 1994 Catalog and Handbook, pages T-155 to T-200, both of which are hereby incorporated by reference). In one embodiment, the carbodimide is first attached to the tag, such as is commercially available for many of the tags described herein.

In one embodiment, ubiquitin moiety is in the form of tag-ubiquitin moiety, wherein, tag is a partner of a binding pair. In one example is the tag is FLAG and the binding partner is anti-FLAG. In this embodiment, a label is attached to the FLAG by indirect labeling. In another embodiment, the label is a label enzyme, which can be, for example, horseradish peroxidase, which is reacted with a fluorescent label enzyme substrate. In one embodiment, the label enzyme substrate is Luminol. Alternatively, the label is a fluorescent label.

In another embodiment, the ubiquitin moiety is in the form of tag-ubiquitin moiety, wherein the tag is a fluorescent label. In one embodiment of interest, the ubiquitin moiety is in the form of tag1-ubiquitin and tag2-ubiquitin, wherein tag1 and tag2 are the members of a FRET pair. In an alternate embodiment, the ubiquitin moiety is in the form of tag1-ubiquitin and tag2-ubiquitin, wherein tag1 is a fluorescent label and tag2 is a quencher of the fluorescent label. In a related embodiment, when the tag1-ubiquitin and tag2-ubiquitin moieties are bound through the activity of a ubiquitin ligase, the tag1 and tag2 are within about 100, 70, 50, 40, or 30 or less angstroms of each other.

In another embodiment, ubiquitin is in the form of tag1,2-ubiquitin and tag1,3-ubiquitin, wherein tag1 is a member of a binding pair, e.g., FLAG, tag2 is a fluorescent label and tag3 is either a fluorescent label such that tag2 and tag3 are members of a FRET pair or tag3 is a quencher of tag2. In one embodiment, one or more amino acids are added to the ubiquitin sequence, using recombinant techniques as described herein, to provide an attachment point for a tag, e.g., a fluorescent label or a quencher. In one embodiment, the one or more amino acids are Cys or Ala-Cys. Preferably, the one or more amino acids are attached to the N-terminal of the ubiquitin. In one exemplary embodiment, the one or more amino acids intervenes the sequence of a FLAG tag and the ubiquitin. In an exemplary embodiment, the tag, e.g., a fluorescent label or a quencher, is attached to the added Cysteine.

Glycosylation Variants and Other Variants

Another type of covalent modification of a polypeptide included within the scope of this invention comprises altering the native glycosylation pattern of the polypeptide. “Altering the native glycosylation pattern” is intended for purposes herein to mean deleting one or more carbohydrate moieties found in native sequence polypeptide, and/or adding one or more glycosylation sites that are not present in the native sequence polypeptide.

Addition of glycosylation sites to polypeptides may be accomplished by altering the amino acid sequence thereof. The alteration may be made, for example, by the addition of, or substitution by, one or more serine or threonine residues to the native sequence polypeptide (for O-linked glycosylation sites). The amino acid sequence may optionally be altered through changes at the DNA level, particularly by mutating the DNA encoding the polypeptide at pre-selected bases such that codons are generated that will translate into the desired amino acids.

Alternatively, the variant may be designed such that the biological activity of the protein is altered. For example, glycosylation sites may be altered or removed. Covalent modifications of polypeptides are included within the scope of this invention. One type of covalent modification includes reacting targeted amino acid residues of a polypeptide with an organic derivatizing agent that is capable of reacting with selected side chains or the N- or C-terminal residues of a polypeptide. Derivatization with bifunctional agents is useful, for instance, for crosslinking a protein to a water-insoluble support matrix or surface for use in the method for screening assays, as is more fully described below. Commonly used crosslinking agents include, e.g., 1,1-bis(diazoacetyl)-2-phenylethane, glutaraldehyde, -hydroxy-succinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1, % octane and agents such as methyl-3-[(p-azidophenyl)dithio]propioimidate. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the ″-amino groups of lysine, arginine, and histidine side chains (Creighton, Proteins: Structure and Molecular Properties, W.H. Freeman & Co., San Francisco, pp. 79-86 (1983)), acetylation of the N-terminal amine, and amidation of any C-terminal carboxyl group.

Further means of increasing the number of carbohydrate moieties on a polypeptide is by chemical or enzymatic coupling of glycosides to the polypeptide. Such methods are described in the art, e.g., in WO 87/05330, and in Aplin and Wriston, CRC Crit. Rev. Biochem., pp. 259-306 (1981). 25 Removal of carbohydrate moieties present on the polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 25952 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzynol., 138:350 (1987). Another type of covalent modification of a protein comprises linking the polypeptide to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192 or 4,179,337.

Candidate Agents

The assays of the invention are designed to identify candidate agents that act as modulators of ubiquitylation of a host cell substrate protein in the present of a viral ubiquitylation modulator protein. By “modulator” is meant a compound which can facilitate an increase or decrease ubiquitylation, with viral proteins that facilitate an increase in ubiquitylated host cell antiviral proteins being of particular interest. The skilled artisan will appreciate that modulators of ubiquitylation may affect activity of a ubiquitylation agent, including activity in transfer or removal of a ubiquitin moiety, interaction between ubiquitin and the substrate, or a combination of these and/or other biological activities related to ubiquitylation.

By “candidate”, “candidate agent”, “candidate modulator”, “candidate ubiquitylation modulator” or grammatical equivalents herein, which terms are used interchangeable herein, is meant any molecule, e.g. proteins (which herein includes proteins, polypeptides, and peptides), small organic or inorganic molecules, polysaccharides, polynucleotides, etc. which are to be tested for ubiquitination modulator activity. Candidate agents encompass numerous chemical classes. In one embodiment, the candidate agents are organic molecules, particularly small organic molecules, comprising functional groups necessary for structural interaction with proteins, particularly hydrogen bonding, and typically include at least an amine, carbonyl, hydroxyl or carboxyl group, usually at least two of the functional chemical groups. The candidate agents often comprise cyclical carbon or heterocyclic structures and or aromatic or polyaromatic structures substituted with one or more chemical functional groups.

Candidate modulators are obtained from a wide variety of sources, as will be appreciated by those in the art, including libraries of synthetic or natural compounds. As will be appreciated by those in the art, the present invention provides a rapid and easy method for screening any library of candidate modulators, including the wide variety of known combinatorial chemistry-type libraries.

In one embodiment, candidate modulators are synthetic compounds. Any number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods. As described in WO 94/24314, one of the advantages of the present method is that it is not necessary to characterize the candidate modulator prior to the assay; only candidate modulators that affect ubiquitylation of a target substrate protein of interest need be identified.

In another embodiment, the candidate modulators are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In one embodiment, candidate modulators include proteins, nucleic acids, and chemical moieties. In one embodiment, the candidate modulators are naturally occurring proteins or fragments of naturally occurring proteins. Thus, for example, cellular extracts containing proteins, or random or directed digests of proteinaceous cellular extracts, may be tested, as is more fully described below. In this way libraries of procaryotic and eucaryotic proteins may be made for screening against any number of ubiquitin ligase compositions. Other embodiments include libraries of bacterial, fungal, viral, and mammalian proteins, with the latter being preferred, and human proteins being especially preferred.

In one embodiment, the candidate modulators are peptides of from about 2 to about 50 amino acids, with from about 5 to about 30 amino acids being usual, and from about 8 to about 20 being particularly of interest. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position.

The synthetic process can be designed to generate randomized proteins or nucleic acids, to allow the formation of all or most of the possible combinations over the length of the sequence, thus forming a library of randomized candidate bioactive proteinaceous agents. A library of all combinations of a peptide 7 to 20 amino acids in length, such as generally proposed herein, has the potential to code for 20⁷ to 20²⁰ different peptides. Thus, with libraries of 10⁷ to 10⁸ different molecules the present methods allow a “working” subset of a theoretically complete interaction library for 7 amino acids, and a subset of shapes for the 20²⁰ peptide library. Thus, in one embodiment, at least 10⁶, 10⁷, or 10⁸. Maximizing library size and diversity is of interest.

In one embodiment, the library is fully randomized, with no sequence preferences or constants at any position. In one embodiment, the library is biased. That is, some positions within the sequence are either held constant, or are selected from a limited number of possibilities. For example, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

A number of molecules or protein domains are suitable as starting points for the generation of biased randomized candidate modulators. A large number of small molecule domains are known, that confer a common function, structure or affinity. In addition, as is appreciated in the art, areas of weak amino acid homology may have strong structural homology. A number of these molecules, domains, and/or corresponding consensus sequences, are known, including, but are not limited to, SH-2 domains, SH-3 domains, Pleckstrin, death domains, protease cleavage/recognition sites, enzyme inhibitors, enzyme substrates, Traf, etc.

As described above generally for proteins, nucleic acid candidate modulator may be naturally occurring nucleic acids, random nucleic acids, or “biased” random nucleic acids. For example, digests of procaryotic or eucaryotic genomes may be used as is outlined above for proteins. Where the ultimate expression product is a nucleic acid, at least 10, at least 12, more usually at least 15, normally at least 21 nucleotide positions need to be randomized, with more preferable if the randomization is less than perfect. Similarly, at least 5, at least 6, more usually at least 7 amino acid positions need to be randomized; again, more are preferable if the randomization is less than perfect. Cyclic polypeptides (see, e.g., Kinsella et al., Biol. Chem. 2002 277:37512-8) are of particular interest as candidate agents (see also U.S. Ser. No. 09/800,770, incorporated by reference herein).

In one embodiment, the candidate modulators are organic moieties. In this embodiment, as is generally described in WO 94/243 14, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

Assay Formats

The invention provides methods for assessing ubiquitylation of a substrate protein in the presence of a retroviral ubiquitylation modulator protein, and further assessing the effect of a candidate agent upon substrate protein ubiquitylation in the presence of the retroviral ubiquitylation modulator protein. In these assays, the influence of candidate agent, the effect of different retroviral ubiquitylation modulator proteins upon different target substrate proteins ubiquitylation enzymes, and/or the susceptibility of different host cell candidate substrate proteins to ubiquitylation modulation in the presence of different retroviral ubiquitylation modulator proteins can be observed and assessed.

In general, the assays of the invention are carried out by bringing into contact (e.g., in a cell or in a suitable cell-free environment) various components of the ubiquitylation cascade so that ubiquitylation of a substrate protein (e.g, CEM15, CD4), and the effect of the candidate agent upon substrate protein ubiquitylation in the presence of a retroviral ubiquitylation modulator protein (e.g., Vif, Vpu), can be assessed.

Identification of Agent that Decrease Ubiquitylation

In one embodiment, the method involves combining (e.g., in a test sample) candidate agent, a substrate protein (e.g., a CEM15 polypeptide, CD4 polypeptide), a ubiquitin activating agent, a ubiquitin conjugating agent, a ubiquitin ligating agent, a ubiquitin moiety and a retroviral ubiquitylation modulating protein (e.g, Vif, Vpu) under conditions suitable for ubiquitylation of the substrate protein. In related embodiments, a de-ubiquitylation agent is also included in the assay. The level of ubiquitylated substrate polypeptide is assessed either qualitatively or quantitatively. A decrease in ubiquitylated substrate polypeptide in the presence of the candidate agent relative to a level in the absence of the candidate agent indicates the agent affects a decrease in ubiquitylation of the substrate protein.

Because the substrate protein is selected to be one that confers antiviral activity upon a host cell, and since ubiquitylation of the substrate protein would otherwise result in its degradation (e.g., via proteosome-mediated degradation), decreasing ubiquitylation of the substrate protein has the effect of enhancing or maintaining the antiviral activity of the substrate protein in a host cell, thus making the environment in a cell less permissive to supporting retroviral replication. Therefore, candidate agents that facilitate a decrease in ubiquitylation of such a substrate protein in essence have activity as antiviral agents against the retrovirus that contains the retroviral ubiquitylation modulator protein.

In related embodiments, the assay uses a tagged ubiquitin moiety (tag-Ub), which can be tagged as discussed above. In another embodiment of particular interest, the substrate protein is CEM15 or CD4. In a further embodiment of particular interest, the retroviral ubiquitylation modulator protein is virion infectivity factor of human immunodeficiency virus (HIV). In related embodiments, the retroviral ubiquitylation modulator protein is Vif or Vpu. Where the retroviral ubiquitin modulator protein is Vif, the cellular substrate is CEM15; where the retroviral ubiquitin modulator protein is Vpu, the cellular substrate is CD4.

In this and further embodiments described below, the ubiquitin activating agent is E1; the ubiquitin conjugating agent is an E2; and/or the ubiquitin ligating agent is an E3. In another exemplary embodiment, the ubiquitin ligating agent is TRAC-1. In another embodiment, where the assay includes a de-ubiquitylation agent, the de-ubiquitylating agent is a USP-25.

Identification of Agent that Increase De-Ubiquitylation

In another assay embodiment, the assay involves identifying candidate agents that enhance activity of a de-ubiquitylation agent so as to provide for enhanced levels of de-ubiquitylated substrate protein. This assay method involves combining (e.g., in a test sample), a candidate agent, a ubiquitylated complex comprising a substrate protein of interest conjugated to a ubiquitin moiety, and a retroviral ubiquitylation modulator protein. De-ubiquitylation of the ubiquitylation complex is then detected. An increase in de-ubiquitylation of the ubiquitylated complex in the presence of the candidate agent relative to de-ubiquitylation of the ubiquitylated complex in the absence of the agent indicates the agent is an antiviral agent for a retrovirus having the retroviral ubiquitylation modulator protein.

In related embodiments, the assay uses a tagged ubiquitin moiety (tag-Ub), which can be tagged as discussed above. Detection of de-ubiquitylation can be accomplished by, for example, detecting released tag-Ub or detecting substrate protein free of the tag of the tag-Ub. In another embodiment of particular interest, the substrate protein is CEM15. In a further embodiment of particular interest, the retroviral ubiquitylation modulator protein is virion infectivity factor of human immunodeficiency virus (HIV). In further related embodiments, the de-ubiquitylating agent is a USP-25 polypeptide.

Deubiquitinating activity, or the modulation of deubiquitinating activity, can be detected and measured using the methods described herein or known in the art. Examples of assays for the detection and measurement of deubiquitinating activity include, but are not limited to, the disappearance of ubiquitinated polypeptides (i.e., ubiquitin complexes), including decrease in the amount of polyubiquitin or ubiquitinated protein or protein remnant or fragment; appearance of intermediate and end products of deubiquitating activity, e.g., the appearance of free ubiquitin monomers or released or cleaved ubiquitin moiety; general or specific protein turnover; binding to ubiquitin moiety; binding to ubiquitinated polypeptides (i.e., ubiquitin complexes); interaction with ATP or cellular components such as trans-acting regulatory factors; and stabilization of specific proteins.

Exemplary assays for detecting agents that enhance de-ubiquitylation of a substrate protein are described in U.S. application Ser. No. 10/232,759, filed Aug. 30, 2002, which application is incorporated herein by reference in its entirety. Further exemplary methods are described in, for example, Sjolander et al. (1991) Anal. chem. 63:2338-2345; Szabo et al. (1995) Curr. Opin. Struct. Biol. 5:699-705; and U.S. Pat. No. 6,329,171 to Kapeller-Libermann et al.; Zhu et al. (1997) Journal of Biological Chemistry 272:51-57, Mitch et al. (1999) American Journal of Physiology 276: C1132-C1138; Liu et al. (1999) Molecular and Cell Biology 19:3029-3038; Ciechanover et al. (1994) The FASEB Journal 8:182-192; Chiechanover (1994) Biol. Chem. Hoppe-Seyler 375:565-581; Hershko et al. (1998) Annual Review of Biochemistry 67:425-479; Swartz (1999) Annual Review of Medicine 50:57-74, Ciechanover (1998) EMBO Journal 17:7151-7160; and D'Andrea et al. (1998) Critical Reviews in Biochemistry; and Molecular Biology 33:337-352).

Agents that Decrease TRAC-1 Activity

In another assay format, the assay involves contacting a candidate with a mammalian cell comprising a TRAC-1 polypeptide and a retroviral ubiquitylation modulator protein under conditions suitable for TRAC-1-mediated ubiquitylation activity, and the effect of the candidate agent upon ubiquitylation activity of TRAC-1 assessed. A decrease in TRAC-1-mediated ubiquitylation in the presence of the candidate agent relative to in the absence of the agent indicates the candidate agent inhibits retroviral-mediated modulation of ubiquitylation.

In related embodiments, the retroviral-mediated ubiquitylation is mediated by human immunodeficiency virus (HIV) virion infectivity factor (Vif). In another related embodiment, retroviral-mediated ubiquitylation is mediated by HIV Vpu. In further embodiments, TRAC-1 activity is detected by, for example, detecting ubiquitylation of a cellular substrate protein (e.g., by detecting incorporated of a tagged ubiquitin moiety) or detecting association of TRAC-1 with a ubiquitylated E2 (which can be ubiquitylated with a tag-Ub). In further related embodiments, the cellular substrate protein is CEM15. In another related embodiment, the cellular substrate protein is CD4.

Identification of Ubiquitylation Agents that Interact with Retroviral Ubiquitylation Modulator Proteins

In another embodiment, the assay methods involve identifying ubiquitylation cascade agents involved in facilitating or inhibiting the effects of a retroviral ubiquitylation modulator protein in ubiquitylation of a host cell substrate protein. This embodiment involves culturing a plurality of cells containing a retroviral ubiquitylation modulator protein, wherein the plurality of cells express a plurality of different ubiquitin agents, where the ubiquitin agent is a ubiquitin moiety, a ubiquitin activating agent, a ubiquitin conjugating agent, a ubiquitin ligating agent, or a de-ubiquitylation agent. Alternatively or in addition, the plurality of cells can contain (e.g., through retroviral infection or by expression of an exogenous polynucleotide) different retroviral ubiquitylation modulator proteins. The plurality of cells are then screened for an altered phenotype.

By “altered phenotype” herein is meant a detectable change in a phenotype of a cell as compared with control cells, e.g. cells not expressing or containing a retroviral ubiquitylation modulator protein or cells not expressing or containing the relevant ubiquitin agent. Detection of a cell having an altered phenotype in the presence of the different ubiquitin agents indicates the ubiquitin agent modulates the phenotype. The cellular phenotype altered can be, for example, permissiveness of the cell to retroviral replication, alteration in levels of ubiquitylation of a host cell substrate protein, particularly a host cell substrate protein that provides an antiviral activity to the cell, and the like. In an embodiment of particular interest, the retroviral ubiquitylation modulator protein is human immunodeficiency virus (HIV) virion infectivity factor (Vif).

In a related embodiment, where the retroviral ubiquitylation modulator protein is varied among the plurality of cells, detection of an altered phenotype indicates the retroviral ubiquitylation modulator protein affects ubiquitylation of a host cell substrate protein.

High-Throughput Assays

In one embodiment, multiple assays are performed simultaneously in a high throughput screening system. In this embodiment, multiple assays may be performed in multiple receptacles, such as the wells of a 96 well plate or other multi-well plate. As will be appreciated by one of skill in the art, such a system may be applied to the assay of multiple candidate agents and/or multiple combinations of ubiquitylation agents and retroviral ubiquitylation modulator protein combinations.

In one embodiment, the present invention is adapted for a high-throughput screening system to detect host cell substrate protein ubiquitylation (e.g., CEM15 ubiquitylation) in the presence of difference combinations of ubiquitin agents (e.g., different ubiquitin moieties, ubiquitin activating agents, ubiquitin ligating agents, ubiquitin conjugation agents, de-ubiquitylation agents) with different retroviral ubiquitylation modulator proteins or candidate retroviral ubiquitylation modulator proteins.

In another embodiment, the present invention is adapted for a high throughput screening system for simultaneously testing the effect of individual candidate agents.

It is understood by the skilled artisan that the steps of the assays provided herein can vary in order. It is also understood, however, that while various options (of compounds, properties selected or order of steps) are provided herein, the options are also each provided individually, and can each be individually segregated from the other options provided herein. Moreover, steps which are obvious and known in the art that will increase the sensitivity of the assay are intended to be within the scope of this invention. For example, there may be additionally washing steps, blocking steps, etc. it is understood that the exemplary embodiments provided herein in no way serve to limit the true scope of this invention, but rather are presented for illustrative purposes. All references cited herein are expressly incorporated by reference in their entirety.

Cell-Free Screening Assays

In general, the method involves combining at least the minimum number of required ubiquitin agents, a retroviral ubiquitylation modulator protein (e.g., Vif) and a substrate protein (e.g, CEM15) assessing either qualitatively or quantitatively a level of ubiquitylation activity. The ubiquitylation substrate protein is a host cell antiviral protein, preferably CEM15. Ubiquitylation can be assessed by detection of mono-ubiquitylation, poly-ubiquitylation, or both.

Assessing ubiquitylation activity can be accomplished in a variety of ways. In general, the assay methods involve combining ubiquitin agents and a retroviral ubiquitylation modulator protein with other components, such as a candidate agent. By “combining” is meant the addition of the various components into a receptacle under conditions in which ubiquitylation (or, in the case of de-ubiquitylation activity assays, de-ubiquitylation) may take place.

In one embodiment, the receptacle is a well of a 96 well plate or other commercially available multiwell plate. In another embodiment, the receptacle is the reaction vessel of a FACS machine. Other receptacles useful in the present invention include, but are not limited to 384 well plates and 1536 well plates. Still other receptacles useful in the present invention will be apparent to the skilled artisan.

The addition of the components may be sequential or in a predetermined order or grouping, as long as the conditions amenable to ubiquitin ligase activity are obtained. Such conditions are well known in the art, and optimization of such conditions is routine in the art.

The components of the present compositions may be combined in varying amounts. In one embodiment, ubiquitin is combined at a final concentration of from to 200 ng per 100 ill reaction solution, preferably at about 100 ng per 100 μl reaction solution. For example, a ubiquitin activating agent (e.g, E1) can be combined at a final concentration of from 1 to 50 ng per 100 μl reaction solution, more preferably from 1 ng to 20 ng per 100 μl reaction solution, most preferably from 5 ng to 10 ng per 100 μl reaction solution. In another example, a ubiquitin conjugating agent (e.g., E2) is combined at a final concentration of 10 to 100 ng per 100 μl reaction solution, more preferably 10-50 ng per 100 μl reaction solution. In another example, a ubit ligating agent (e.g., E3) is combined at a final concentration of from 1 ng to 500 ng per 100 μl reaction solution, more preferably from 50 to 400 ng per 100 μl reaction solution, still more preferably from 100 to 300 ng per 100 μl reaction solution, most preferably about 100 ng per 100 μl reaction solution.

The components of the invention are combined under reaction conditions that favor ubiquitylation activity (e.g., ubiquitin ligase activity and/or de-ubiquitylation activity). Generally, this will be physiological conditions. Incubations may be performed at any temperature which facilitates optimal activity, typically between 4 and 40° C. Incubation periods are selected for optimum activity, but may also be optimized to facilitate rapid high through put screening. Typically between 0.5 and 1.5 hours will be sufficient.

A variety of other reagents may be included in the compositions. These include reagents like salts, solvents, buffers, neutral proteins, e.g. albumin, detergents, etc. which may be used to facilitate optimal ubiquitylation enzyme activity and/or reduce non-specific or background interactions. Also reagents that otherwise improve the efficiency of the assay, such as protease inhibitors, nuclease inhibitors, anti-microbial agents, etc., may be used. The compositions can also include adenosine tri-phosphate (ATP).

The mixture of components may be added in any order that promotes ubiquitylation or de-ubiquitylation as appropriate, or optimizes identification of candidate modulator effects. In one embodiment, ubiquitin is provided in a reaction buffer solution, followed by addition of the ubiquitylation enzymes. In an alternate embodiment, ubiquitin is provided in a reaction buffer solution, a candidate modulator is then added, followed by addition of the ubiquitylation enzymes.

In one example, at least one of the components is immobilized on a substrate, e.g., the ubiquitin ligating agent (e.g., E3). Binding of assay components may be done directly or indirectly (e.g., via covalent or non-covalent binding to a component which is bound to the substrate). Binding of the component can be via a tag moiety, which may or may not provide a detectable signal. In exemplary methods, a ubiquitin ligating agent (e.g., E3) is bound to a surface substrate. In another embodiment, ubiquitin conjugating agent (e.g., E2) is bound to a surface substrate. In general, any substrate binding molecule can be used.

As will be appreciated by those of skill in the art, the surface substrate binding element and substrate to which the element binds can be selected according to the design of the assay and the desired characteristics, e.g., an element-substrate combination that will be effective for facilitating the separation of bound and unbound ubiquitin. The substrate used in embodiments involving immobilization of an assay component can be any suitable substrate, e.g., a well of a multi-well plate, a bead, and the like.

In another embodiment, the ubiquitin agents and other assay components are free in solution. In this embodiment, ubiquitylation activity can be monitored using a system that produces a signal which varies with the extent of ubiquitylation, such as the fluorescence resonance energy transfer (FRET) system described in detail below. In one embodiment, the ubiquitin is labeled, either directly or indirectly, as further described below, and the amount of label is measured. This allows for easy and rapid detection and measurement of ligated ubiquitin, making the assay useful for high-throughput screening applications. In one embodiment, the signal of the label varies with the extent of ubiquitylation, such as in the FRET system described below. One of ordinary skill in the art will recognize the applicability of the present invention to screening for agents which modulate ubiquitylation.

In a related embodiment, the assay composition comprises tag1-ubiquitin, tag2-ubiquitin, E1, E2 and E3. In one embodiment, tag1 and tag2 are labels, preferably fluorescent labels, most preferably tag1 and tag2 are a FRET pair. In this embodiment, ubiquitylation is measured by measuring the fluorescent emission spectrum. This measuring may be continuous or at one or more times following the combination of the components. Alteration in the fluorescent emission spectrum of the combination as compared with unligated ubiquitin indicates the amount of ubiquitylation. The skilled artisan will appreciate that in this embodiment, alteration in the fluorescent emission spectrum results from ubiquitin bearing different members of the FRET pair being brought into close proximity, either through the formation of poly-ubiquitin and/or by binding nearby locations on a protein, preferably a target protein

Detection of Ubiquitylation Activity

Once combined, the level of ubiquitylation activity can be assessed in a variety of ways. For example, the level of ubiquitylated substrate protein and/or the degree of ubiquitylation of the substrate protein can be assessed; the level of free ubiquitin can be assessed; the association of substrate protein with a ubiquitin conjugating agent; association of a substrate protein, ubiquitin conjugating agent, and ubiquitin ligating agent; and other variations that will be readily appreciated by the ordinarily skilled artisan. As will also be apparent to the skilled artisan, the detection of bound ubiquitin bound will encompass not only the particular ubiquitin bound directly to the corresponding protein (e.g., ubiquitin activating agent, ubiquitin conjugating agent, ubiquitin ligating agent, and/or substrate protein), but also the ubiquitin proteins bound in a polyubiquitin chain. In one embodiment, the assay is conducting by assessing ubiquitin ligase activity as described in PCT Publication No. WO 01/75145, which application is incorporated by reference herein in its entirety.

In one embodiment, ubiquitylation is measured, which can be accomplished by, for example, detection of a tag attached to the ubiquitin moiety, e.g., a fluorescent label. In another embodiment, the tag attached to the ubiquitin moiety is an enzyme label or a binding pair member which is indirectly labeled with an enzyme label. In this latter embodiment, the enzyme label substrate produces a fluorescent reaction product. In either of these embodiments, the amount of ubiquitin bound is measured by luminescence. As used herein, “luminescence” or “fluorescent emission” means photon emission from a fluorescent label. In an embodiment where FRET pairs are used, fluorescence measurements may be taken continuously or at time-points during the ligation reaction. Equipment for such measurement is commercially available and easily used by one of ordinary skill in the art to make such a measurement.

Other modes of measuring bound ubiquitin are well known in the art and easily identified by the skilled artisan for each of the labels described herein. For instance, radioisotope labeling may be measured by scintillation counting, or by densitometry after exposure to a photographic emulsion, or by using a device such as a PhosphorImager. Likewise, densitometry may be used to measure bound ubiquitin following a reaction with an enzyme label substrate that produces an opaque product when an enzyme label is used.

In one embodiment, the assay is conducted to detect ubiquitin ligase activity. In this embodiment, the assay can be performed by adapting the assays described in PCT Publication No. WO 01/75145, which describes assay for detecting ubiquitin ligase activity, including such assays conducted in a cell-free environment.

Cell-Based Assays

In one embodiment, the assay is conducted in a cell, preferably a mammalian cell, more preferably in a mammalian cell susceptible to retroviral infection and/or permissive to retroviral replication.

In this embodiment, the ubiquitin agents, retroviral ubiquitylation modulator protein, and substrate protein are provided in a host cell, e.g., by expression of an endogenous or exogenous nucleic acid encoding the polypeptides, or by introduction of the polypeptides by, e.g., viral delivery. The retroviral ubiquitylation modulator protein can be introduced by viral infection with a wild-type or modified virus so as to introduce a functional retroviral ubiquitylation modulator protein in the cell.

Where co-expression of assay components is desired, co-expression may be achieved by introducing into the cell a vector comprising nucleic acids encoding two or more of the assay components, or by introduction of separate vectors, each comprising a single component of the desired assay components. In one embodiment, the candidate agents are peptides, e.g., randomized peptides, which can also be expressed in the host cell.

Host Cells for Cell-Based Assays

In general, the host cells used in cell-based assays of the invention are cells expressing the host cell substrate of interest (e.g., CEM15) and containing a retroviral ubiquitylation modulator protein (e.g., Vif). The retroviral ubiquitylation modulator protein can be present in the cell as a result of retroviral infection or by, for example, introduction of and expression of an exogenous polynucleotide encoding the retroviral ubiquitylation modulator protein.

The host cell substrate of interest can be either an endogenous host cell protein, or can be present in the cell as a result of, for example, introduction of and expression of an exogenous polynucleotide encoding the substrate protein of interest.

Mammalian cells, particularly human cells, are of particular interest. Where mammalian cells are used, essentially any mammalian cells can be used, with mouse, rat, primate and human cells being particularly preferred.

Accordingly, suitable cell types include, but are not limited to, cells that are capable of supporting retroviral replication and, in some embodiments, capable of being infected by the relevant retrovirus. Where the assay is designed to identify agents that modulate ubiquitylation of a substrate protein which in turn affects viral replication (e.g., CEM15), the cell need not be one that is susceptible to infection, only one that supports retroviral replication. Where the cell used is susceptible to infection, the cell is one that expresses on its surface the requisite receptors or other cell surface protein(s) required for viral entry (e.g., CD4 and CCR5 (or CXCR5) for HIV).

The cell is also selected for expression of—or is modified to express—the substrate protein of interest. In one embodiment, the host cell substrate protein is CEM15. Where the CEM15 expressed is an endogenous CEM15, host cells expressing an endogenous CEM15 are suitable for use., with T cells, especially CD4+ T cells being of particular interest. In another embodiment, the substrate protein is CD4; where the CD4 is to be endogenously expressed, the host cell is, for example a T cell, a B cell, or other immune cell where the cell can be a primary cell or cell line. Alternatively, the cells may further comprise a nucleic acid, e.g., an exogenous nucleic acid, e.g., a recombinant nucleic acid, that encodes a target protein.

The mammalian cell for use as a host cell should be selected according to its permissiveness in supporting replication of the retrovirus of interest. In short, test cells should be permissive (i.e., support or allow retroviral replication) so that agents that decrease permissiveness can be identified. Non-permissive or semi-permissive cells can be used as controls as appropriate.

For example, where the retroviral ubiquitylation modulator protein is Vif, the mammalian host cell is one that requires Vif to be present in order to support replication of the immunodeficiency virus (e.g., HIV). Vif is not necessarily required for replication of HIV-1 in all cells, but is required for HIV-1 replication in primary T lymphocytes and monocytes/macrophages. The differences in requirement for of Vif for replication has led to categorization of primary cells or cell lines as either non-permissive (H9, HYT78, A3.0, primary CD4+ T-cells), semi-permissive (CEM-ss, monocyte derived macrophages (MDM)) or permissive (primary T lymphocytes and monocytes/macrophages, HeLa, 293T, Cos-7, SupT1) for replication of Vif-defective viruses (see, e.g., Sova et al. J Viol 67:6322-6366 (1993); Zhang et al. J virol 74:8252-8261 (2000)).

Assay Designs

The ordinarily skilled artisan will appreciate that various assay designs with respect to the assay component and to the methods of detection of ubiquitylation activity described above can be readily adapted for implementation in a cell-based assay.

In one embodiment, the assay is conducting by assessing ubiquitin ligase activity as described in PCT Publication No. WO 01/75145, which application is incorporated by reference herein in its entirety. Further methods for assessing ubiquitylation activity (e.g., using functional assays) are described in U.S. application serial no. 10/232,951, filed Aug. 30, 2002, and in PCT application serial no. PCT/US03/026843, filed Aug. 29, 2003, each of which applications is incorporated herein by reference in its entirety. Methods for detecting de-ubiquitylation activity are described in, for example, U.S. application Ser. No. 10/232,759, filed Aug. 30, 2002, which application is incorporated herein by reference in its entirety.

In general, cell-based assays involve contacting a cell containing the assay components with a candidate agent, and culturing the cell for a suitable period and under suitable conditions to allow for ubiquitylation/de-ubiquitylation activity to occur with respect to the substrate protein. The ordinarily skilled artisan will appreciate that precise culture methods will vary according to, for example, the host cell used, and is susceptible to ready optimization. Methods and means for detecting ubiquitylation activity can be adapted from those described above for cell-free assays.

For example, in an representative embodiment a cell based screen employs a mammalian cell in which an apobec3G-reporter fusion protein (e.g., an apobec3G-luciferase or GFP fusion protein) and vif are co-expressed. Ubiquitin-mediated degradation of the apobec3G-reporter fusion protein can be evaluated by assessing the reporter signal in the presence and absence of candidate agents, e.g., cyclic peptides, as discussed above. Agents can be screened for anti-vif activity in such as assay.

In one embodiment, the assay is designed so as to be readily amenable for use in high-throughput assays. Preferably, in this embodiment, ubiquitylation activity can be detected without the need for isolation of, for example, ubiquitylated substrate protein or lysis of the host cell. For example, the FRET embodiment can be employed so that a level of ubiquitylation activity can be readily associated with a detectable signal that can be extrapolated to a level of ubiquitylation activity. For example, the intensity of the detectable signal can be associated with a level of ubiquitylation activity in the cell.

The cells can be cultured in any suitable receptacle, preferably in a receptacle that is amenable for high throughput assays (e.g., a multi-well plate).

Combinatorial Assays

In certain embodiments, the assays are adapted to examine the combinatorial relationships between the different ubiquitin agents, different host cell substrate proteins, and/or different retroviral ubiquitylation modulator proteins. Accordingly, the present invention involves functional ubiquitylation screens. The methods include providing a cell culture, whose cells contain a library of nucleic acids comprising nucleic acids encoding variant ubiquitin agents such as ubiquitin activating, ubiquitin conjugating or ubiquitin ligating agents. The invention further provides screening the cell culture for altered phenotype as compared to control cells, isolating those with altered phenotypes and identifying the variant ubiquitin agent(s) that resulted in the altered phenotype.

In one embodiment, the invention provides culturing cells expressing different ubiquitin agents and assaying a functional readout for the activity of the ubiquitin agents. Modulation of the functional assay indicates involvement of the ubiquitin agent in that pathway.

In general, the methods involve expressing a ubiquitin moiety, a retroviral ubiquitylation modulator protein, and one or more ubiquitin agents in a cell system, and determining the effect of the ubiquitin moiety, ubiquitin agent or variant of the ubiquitin moiety or ubiquitin agent in a functional assay. The functional assay may involve a cellular readout, or may involve determining the amount of ubiquitin on a target protein. That is, the method involves measuring the amount of ubiquitin moiety attached to at least one of the following substrate molecules: a ubiquitin agent; a target protein; or a mono- or poly-ubiquitin moiety which is preferably attached to a ubiquitin agent or target protein.

Accordingly, the invention can involve assays using a plurality of cells having a plurality of different ubiquitin moieties, a plurality of cells having a plurality of different ubiquitin activating agents, a plurality of cells having a plurality of different ubiquitin conjugating agents, a plurality of cells having a plurality of different ubiquitin ligating agents, a plurality of cells having a plurality of different target substrate proteins, and/or a plurality of cells having a plurality of different retroviral ubiquitylation modulator proteins. These various libraries of pluralities of cells can be used in assays as described herein to determine, for example, the effect of the differing ubiquitin agent, different retroviral ubiquitylation modulator protein, and/or different candidate agent upon ubiquitylation activity in the host cell.

In Vivo Screening

Assays of the invention can be adapted so that they can be conducted in a non-human animal model. In addition, agents identified as having a desired activity in a cultured cell-based assay can be further tested in a non-human animal model. Non-human animals models for retroviral infection are known in the art. For example simian immunodeficiency virus infection of monkeys can serve as a model system for the study of AIDS pathogenesis, treatment, and prevention (see, e.g., Hirsch et al. Adv Pharmacol. 2000;49:437-77).

Hollow fiber-based assays, which involve use of retrovirally infected cells in a hollow fiber implanted in a non-human animal, are described in the art (see, e.g., Dursano et al. “Pharmacodynamics of abacavir in an in vitro hollow-fiber model system,” Antimicrob Agents Chemother. 2002 February;46(2):464-70; Drusano et al. “Hollow-fiber unit evaluation of a new human immunodeficiency virus type 1 protease inhibitor, BMS-232632, for determination of the linked pharmacodynamic variable.”, J Infect Dis. 2001 Apr. 1;183(7):1126-9. Epub 2001 Mar. 1; Rana et al. “Intracellular phosphorylation of zidovudine in an in vitro hollow fiber model.” Pharmacotherapy. 1999 August;19(8):979-83; Quenelle et al. “Evaluation of anti-AIDS drugs in conventional mice implanted with a permeable membrane device containing human T cells infected with HIV.” Antiviral Res. 1997 July;35(2):123-9; Hollingshead et al. “In vivo drug screening applications of HIV-infected cells cultivated within hollow fibers in two physiologic compartments of mice.” Antiviral Res. 1995 November;28(3):265-79; Bilello et al. “Effect of 2′,3′-didehydro-3′-deoxythymidine in an in vitro hollow-fiber pharmacodynamic model system correlates with results of dose-ranging clinical studies.” Antimicrob Agents Chemother. 1994 June;38(6):1386-91.) Hollow fiber assays can be used in vivo assays based on the assays described above. For example, the host cells described above can be placed in a hollow fiber, implanted into a non-human animal host (e.g., rat or mouse), the candidate agent administered, and the effects upon the cells in the hollow fiber and/or viral infection evaluated. Hollow fiber models can also be used to further screen agents identified by having a desired activity in the assays described above.

Kits

Also provided are reagents and kits thereof for practicing one or more of the above-described methods. The subject reagents and kits thereof may vary greatly. Typically, the kits at least include a the minimal ubiquitin agents, a ubiquitylation substrate protein, and a retroviral ubiquitylation modulator protein, wherein at least one of these components comprises a tag adapted to facilitate detection of ubiquitylation activity. The subject kits may also include one or more additional reagents, e.g., reagents employed in detecting the tag.

In addition to the above components, the subject kits can further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Methods of Inhibiting HIV Replication

In another aspect, the invention features methods of inhibiting retroviral replication in a cell by affecting a particular selected ubiquitin agent of the ubiquitylation cascade.

In one embodiment, replication of a retrovirus in a host cell is inhibited by contacting a mammalian cell infected with a retrovirus with an agent that inhibits ubiquitylation activity of E1 in the infected cell, where the agent is provided in amount effective to inhibit replication of the retrovirus in the cell. In related embodiments, the retrovirus is human immunodeficiency virus (HIV), e.g., HIV-1. In a related embodiment, the retrovirus delivers virion infectivity factor (Vif) into the host cell.

Exemplary ubiquitination-inhibitory agents employable in the subject methods include those described in: PCT/US03/36747, filed Nov. 13, 2003 “Rhodanine Derivatives and Pharmaceutical Compositions Containing Them” (atty. docket no. P.0131.03.WO); U.S. Ser. No. 10/858,537, filed Jun. 1, 2004 “Ubiquitin Ligase Inhibitors” (atty. docket no. P.0137.02.US); U.S. patent application Ser. No. ______ entitled “BENZOTHIAZOLE COMPOSITIONS AND THEIR USE AS UBIQUITIN LIGATION INHIBITORS”, filed Oct. 18, 2004 (atty. docket no. P.0152.02.US); U.S. patent application Ser. No. ______ entitled RHODANINE COMPOSITIONS FOR USE AS ANTIVIRAL AGENTS, filed Oct. 28, 2004 (atty. docket no. P.0154.02.US; and U.S. patent application 60/582,261, filed Jun. 22, 2004 “Ubiquitin Ligase TRAF6 Inhibitors” (atty. docket no. P.0163.00.US). Each of the aforementioned patent application is specifically incorporated by reference in their entirety for all purposes.

The invention also provides methods for inhibiting retroviral replication in a host cell by contacting the infected host cell with an agent that inhibits TRAC-1-mediated ubiquitylation in the infected cell., where the agent is provided in amount effective to inhibit replication of the retrovirus in the cell. In related embodiments, the retrovirus is human immunodeficiency virus (HIV), e.g., HIV-1. In a related embodiment, the retrovirus delivers virion infectivity factor (Vif) into the host cell.

The invention also provides a method for inhibiting retroviral replication in an infected host cell by contacting the cell with an agent that promotes USP-25-mediated de-ubiquitylation of CEM15 in the infected cell, where the agent is provided in an amount effective to enhance USP-25-mediated de-ubiquitylation and inhibit retroviral replication in the cell. In related embodiments, the retrovirus is human immunodeficiency virus (HIV), e.g., HIV-1. In a related embodiment, the retrovirus delivers virion infectivity factor (Vif) into the host cell.

In embodiments related to each of the above, the agent is one that, regardless of the actual ubiquitin agent targeted by the agent, ultimately inhibits ubiquitylation of CEM15 polypeptide in the cell. For example, the agent inhibits E1-mediated activation of a ubiquitin conjugating agent that, with a ubiquitin ligating agent (e.g., E3) facilitates ubiquitylation of CEM15 in the cell. In another example, the ubiquitin conjugating agent involved in the E1-meidated ubiquitylation cascade is TRAC-1. In yet another example, the agent promotes or enhances USP-25-mediated de-ubiquitylation of CEM15.

In general, in each of the methods described above for inhibiting retroviral replication, the agent is administered so that it comes into contact with retrovirally-infected cells.

Subjects to be Treated

Any subject having a retroviral infection may be treated according to the invention. Mammalian subjects, especially human subjects, are of particular interest. The terms “individual,” “host,” “subject,” and “patient,” used interchangeably herein, refer to a mammal, including, but not limited to, murines, simians, humans, mammalian farm animals, mammalian sport animals, and mammalian pets.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The subjects to be treated thus include those having or at risk of retroviral infection. The subjects may be symptomatic or asymptomatic. Diseases and symptoms associated with retroviral infection include, but are not limited to immunodeficiency, cytopathic responses, leukemogenesis, and other clinical pathologies and symptoms. The methods of the invention can be continued until a desired clinical endpoint is attained (e.g., symptoms diminish or are otherwise improved, viral clearance (e.g. as detected by a decrease in viral titer or undetectably viral titer, etc.).

Formulations and Routes of Administration

Antiviral agents suitable for use in the invention in the methods of inhibiting retroviral replication (referred to herein as “the agents” or “the active agents” for convenience) as described herein can be formulated in a variety of ways suitable for administration. In general, these compounds are provided in the same or separate formulations in combination with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the agents are formulated separately or in combination, e.g., in an aqueous or non-aqueous formulation, which may further include a buffer. Suitable aqueous buffers include, but are not limited to, acetate, succinate, citrate, and phosphate buffers varying in strength from 5 mM to 100 mM. In some embodiments, the aqueous buffer includes reagents that provide for an isotonic solution. Such reagents include, but are not limited to, sodium chloride, and sugars e.g., mannitol, dextrose, sucrose, and the like. In some embodiments, the aqueous buffer further includes a non-ionic surfactant such as polysorbate 20 or 80.

Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In the subject methods, the active agents may be administered to the host using any convenient means capable of resulting in the desired therapeutic effect. Thus, the agents can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, agents may be administered in the form of their pharmaceutically acceptable salts, or they may also be used alone or in appropriate association, as well as in combination, with other pharmaceutically active compounds. The following methods and excipients are merely exemplary and are in no way limiting.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature. Agents can also be provided in sustained release or controlled release formulations, e.g., to provide for release of agent over time and in a desired amount (e.g., in an amount effective to provide for a desired therapeutic or otherwise beneficial effect).

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of the agents calculated in an amount sufficient to produce the desired effect in association with a pharmaceutically acceptable diluent, carrier or vehicle. The specifications for the unit dosage forms for use in the present invention depend on the particular compound employed and the effect to be achieved, the pharmacodynamics associated with each compound in the host, and the like.

Dosage forms of particular interest include those suitable to accomplish intravenous or oral administration, as well as dosage forms to provide for delivery by a nasal or pulmonary route (e.g., inhalation), e.g., through use of a metered dose inhaler and the like.

In general, agents for use in the invention is formulated in either parenteral or enteral forms, usually enteral formulations, more particularly oral formulations. Agents for use in the invention are formulated for parenteral administration, e.g., by subcutaneous, intradermal, intraperitoneal, intravenous, or intramuscular injection. Administration may also be accomplished by, for example, enteral, oral, buccal, rectal, transdermal, intratracheal, inhalation (see, e.g., U.S. Pat. No. 5,354,934), etc.

The invention also contemplates administration of additional agents with the antiviral agents according to the invention, such as other antiviral agents that work through the same of different mechanism.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Cellular Assays for Evaluating Vif Activity

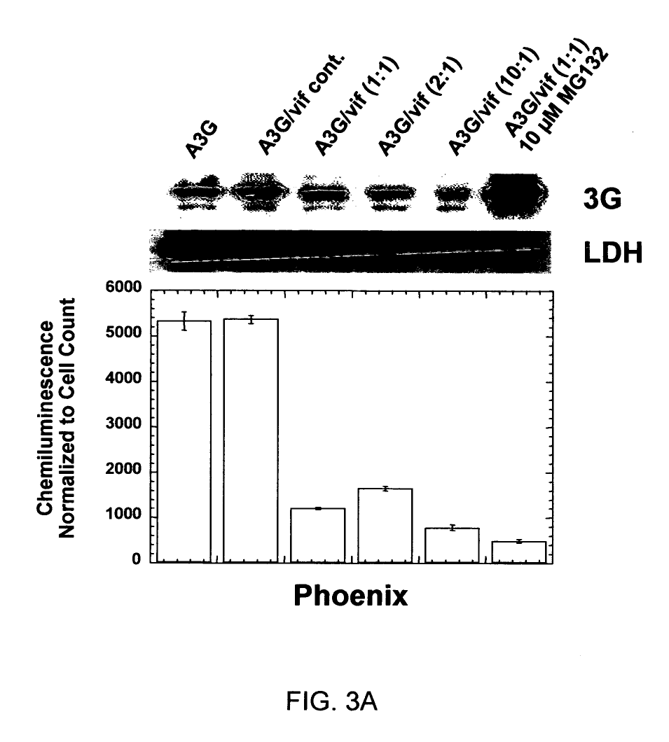

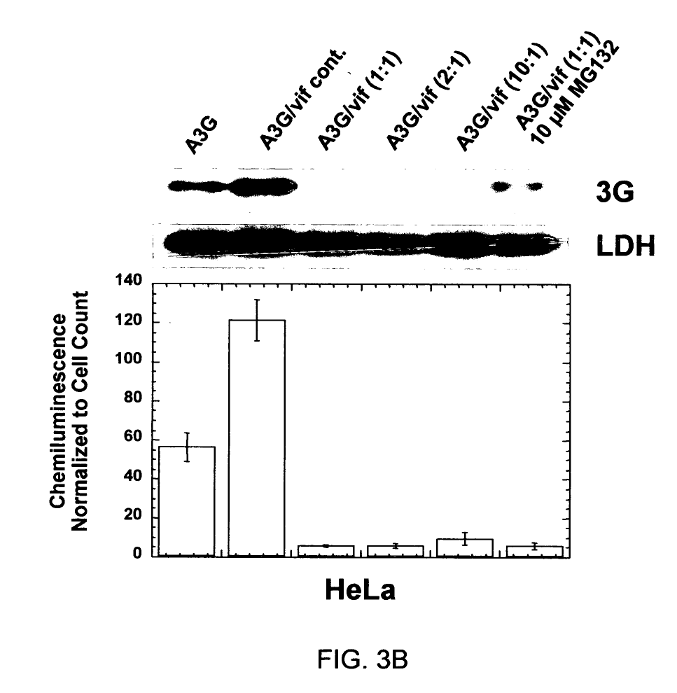

A construct encoding a luciferase/apobec3G fusion protein was introduced into Phoenix and HeLa cells and transient expression of the luciferase/apobec3G fusion protein was monitored by detecting chemiluminescence. Results were normalized to the number of cells assayed. FIGS. 3A and 3B show that the luciferase/apobec3G fusion protein (indicated by “A3G”) is readily expressed in mammalian cells. The gels shown at the top of both FIGS. 3A and 3B show results obtained from western blots of cell extracts using antibodies. Tesults obtained using anti-apobec3G and anti-lactate dehydrogenase antibodies are indicated as “3G” and “LDH”, respectively.

Co-introduction of a second construct encoding vif at relative ratios of 1:1, 2:1, 10:1 into luciferase/apobec3G-containing cells causes a dramatic decrease in luciferase activity, demonstrating that vif modulates ubiquitynation and subsequent degradation of the luciferase/apobec3G fusion protein. This phenomenon is inhibited by adding MG132, a known inhibitor of proteosome-mediated protein degradation (see the band observed in the final lane of each of the western blots). Cells containing both vif and a luciferase/apobec3G reporter can therefore be used to test agents for vif-inhibitory activity.

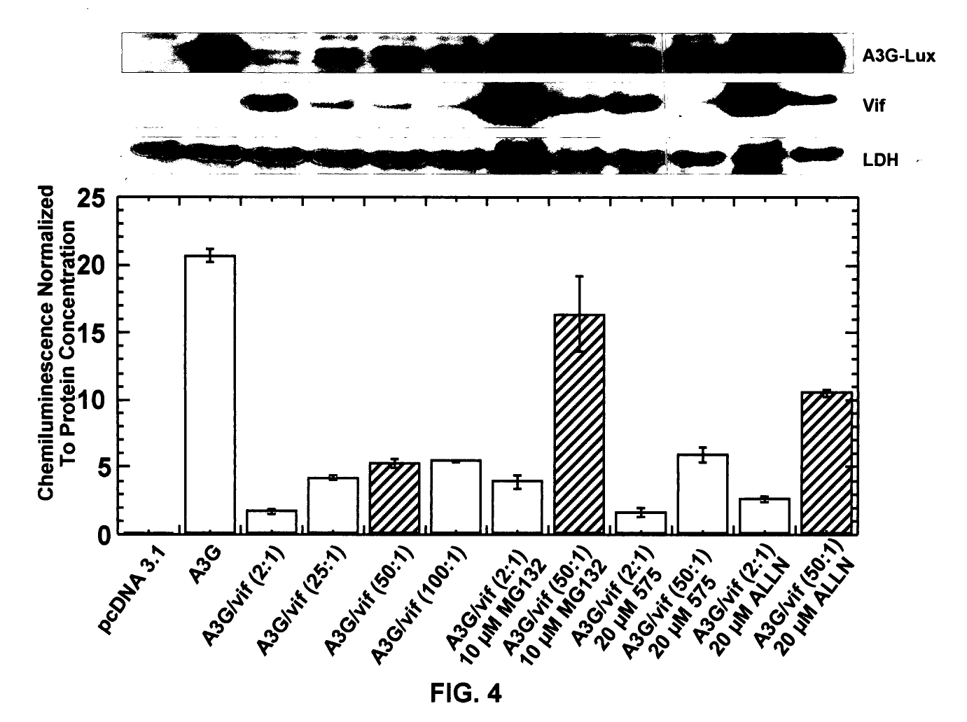

Cells were co-transfected with the luciferase/apobec3G and vif constructs at relative concentrations of 2:1, 25:1, 50:1 and 100:1 (luciferase/apobec3G:vif) in the presence or absence of 10 μM or 20 μM MG132 or ALLN, known inhibitors of proteosome-mediated protein degradation. As shown in FIG. 4, MG132 and ALLN maximally increased luciferase activity when the relative concentrations of the luciferase/apobec3G constructs were 50:1 (data indicated by the hatched bars).

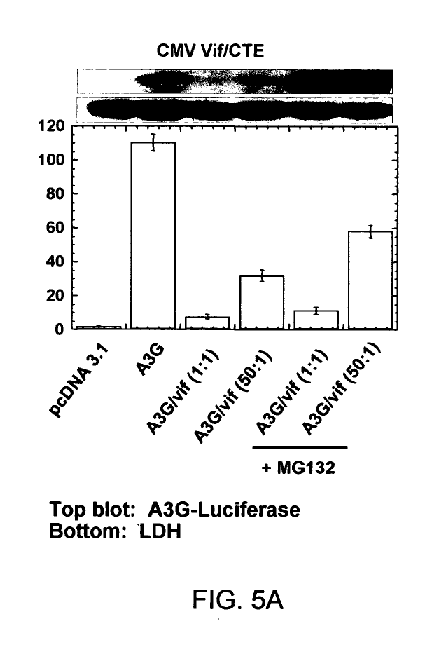

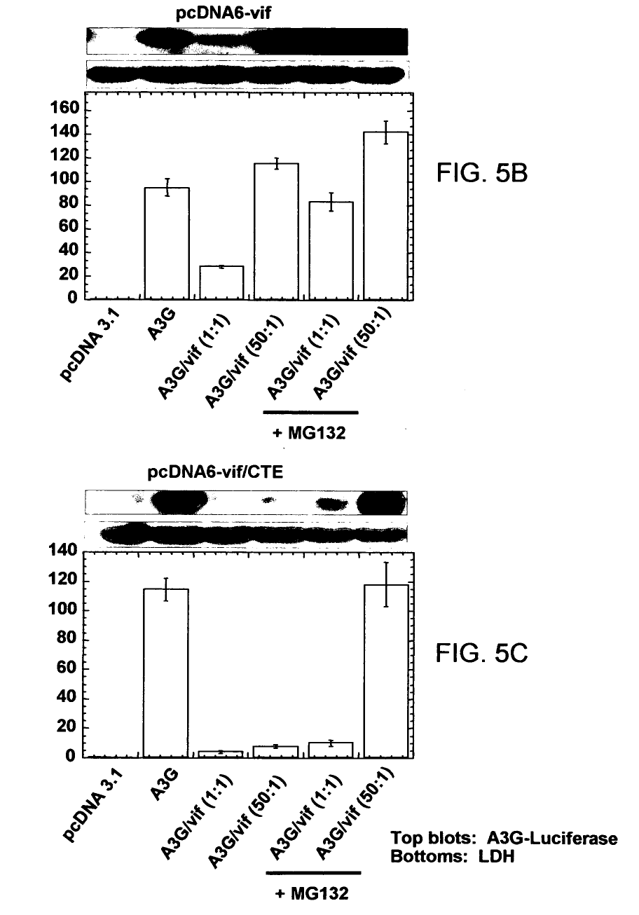

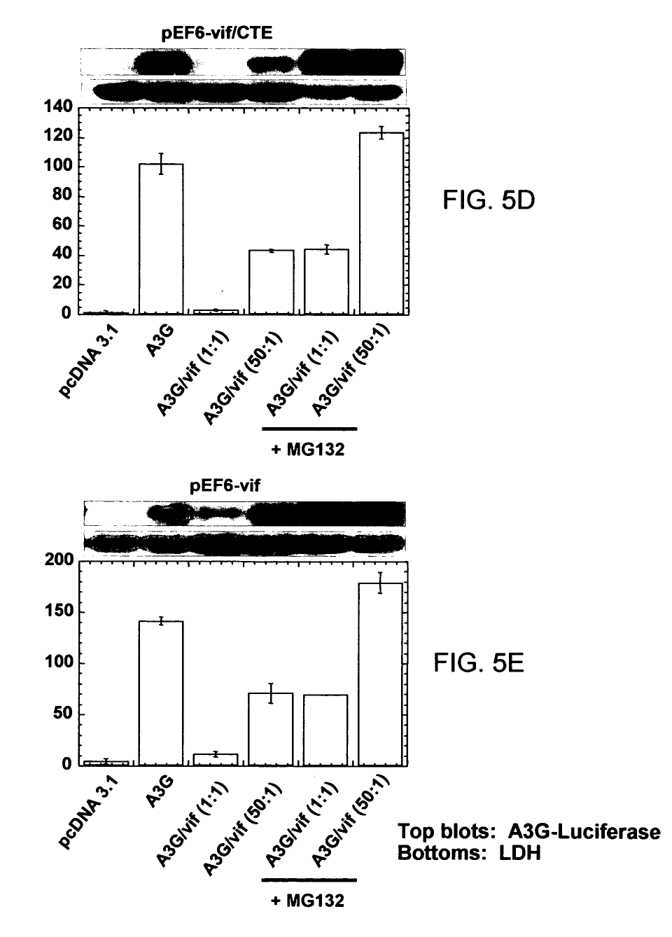

Several vectors, including pCMV, pcDNA6, pcDNA6 containing a constitutive transport element (CTE), pEF6 and pEF6 containing a CTE were tested to determine if they were suitable for co-expressing vif with luciferase/apobec3G in a cell, and whether the vif activity encoded by those constructs could be modulated by ubiquitinylation inhibitors. FIGS. 5A-5E show that all constructs were suitable for co-expressing vif with luciferase/apobec3G in a cell, and vif activity in those cells could be modulated by MG132.

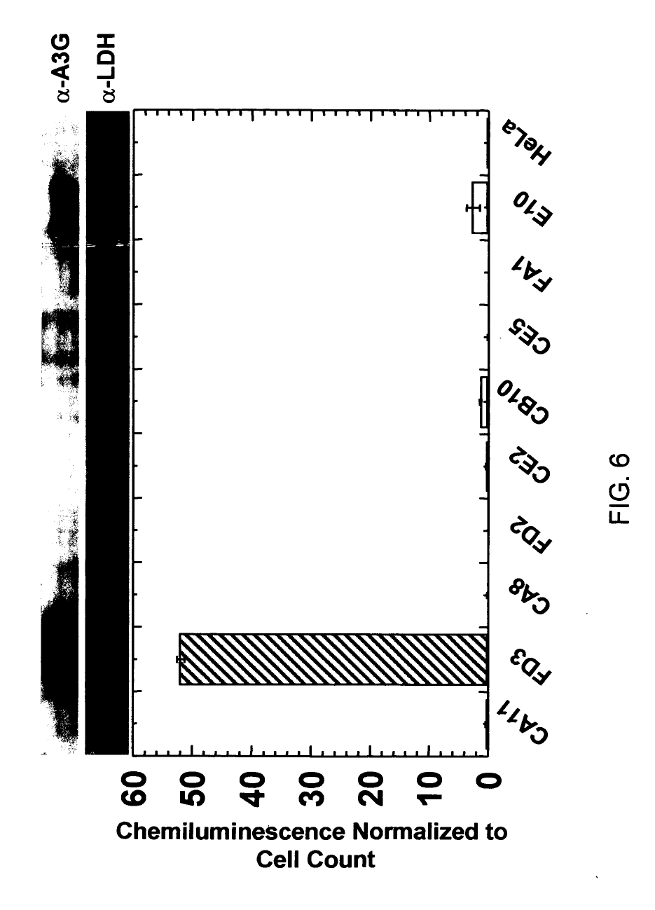

HeLa cells were stably transfected with a retroviral construct encoding the luciferase/apobec3G fusion protein, and a luminescent cell line (line FD3) was selected for vif activity assays. The graph of FIG. 6 shows that FD3 produces detectable levels of the luciferase/apobec3G protein.

### Example 2

### Cell-Free Assays for Evaluating Vif Activity

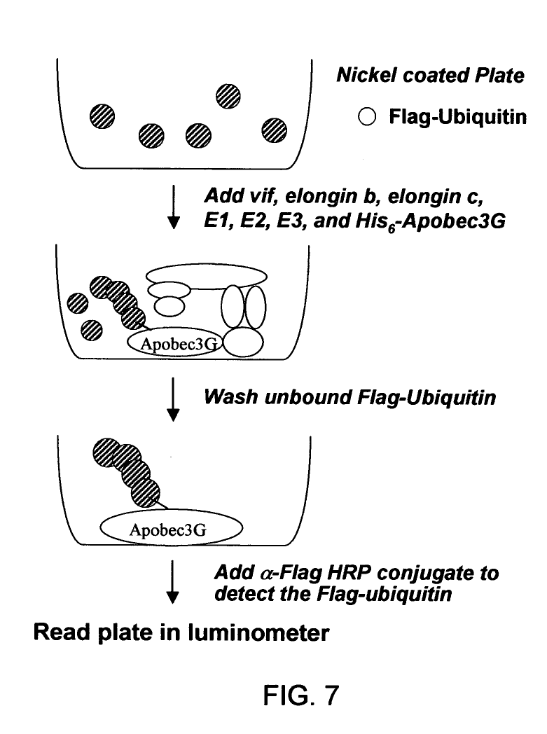

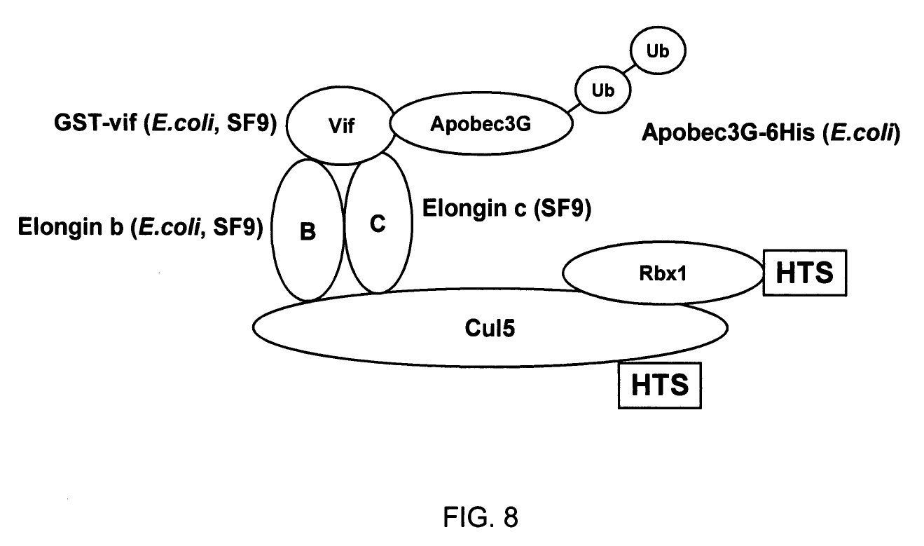

FIG. 7 shows a schematic representation of a cell-free method for measuring vif activity that employs a Flag-tagged ubiquitin, vif, his-tagged apobec3G and other assay components. The expression system used to produce those components is shown in FIG. 8.

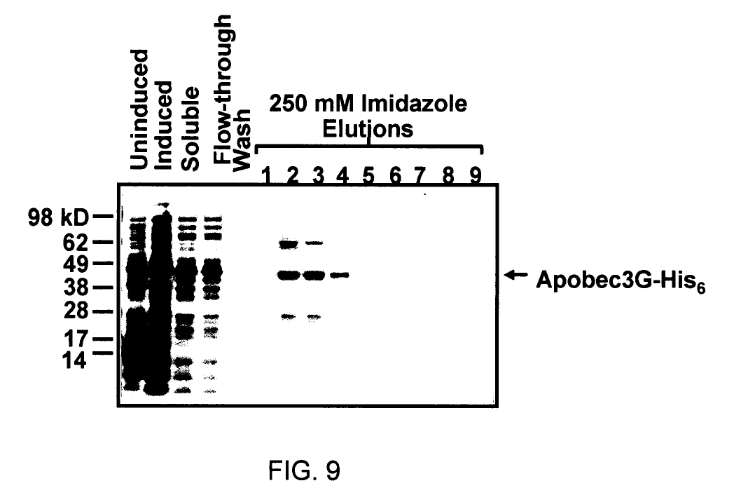

A gel showing production and purification of his-tagged apobec3G is shown in FIG. 9. BL21(DE3) cells were transformed by pET27b:Apobec3G. Bacteria were grown at 37° C. until OD600 reached ˜0.7, induced using 1 mM IPTG, and then transferred to 18° C. shaker and grown overnight.

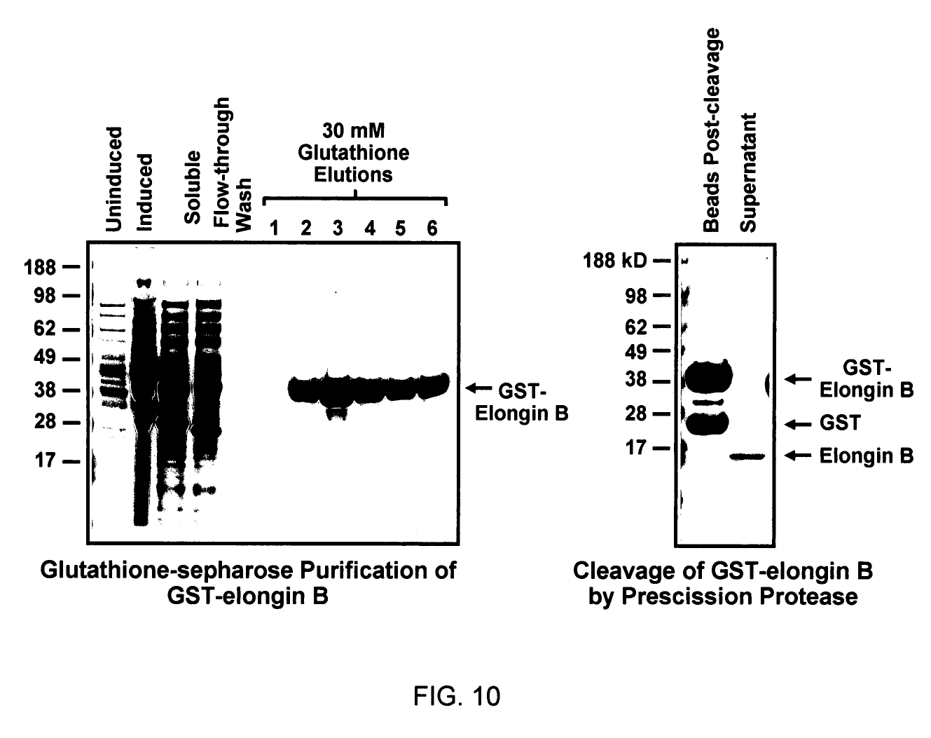

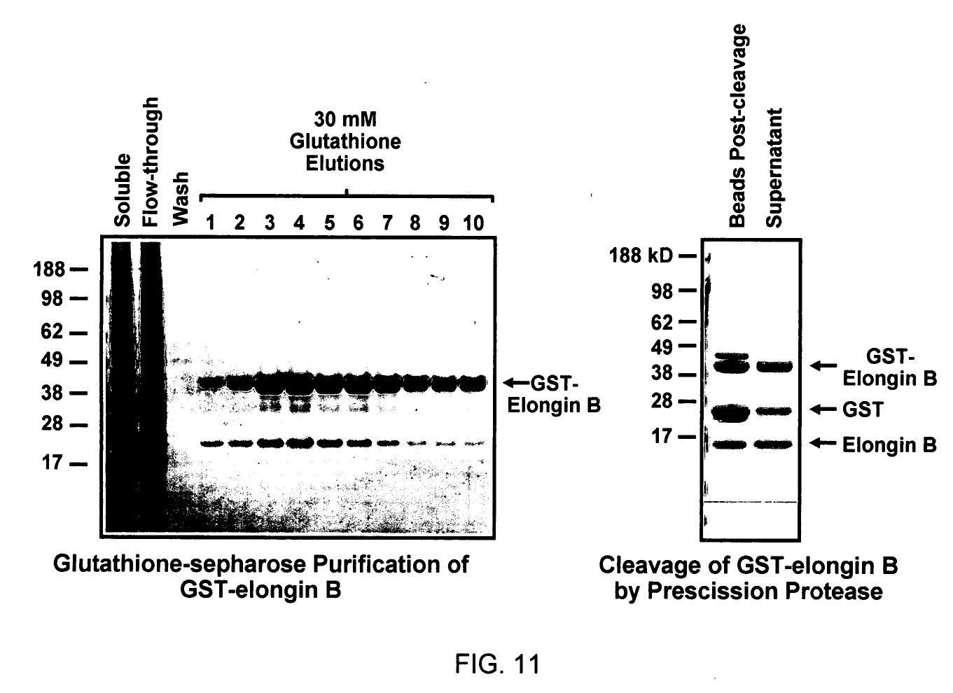

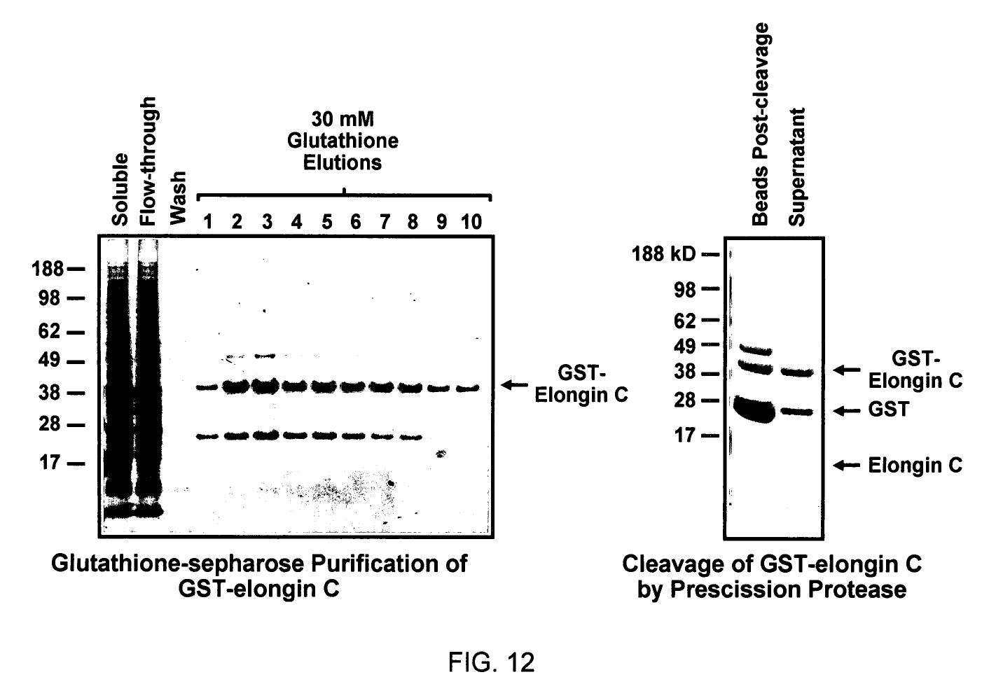

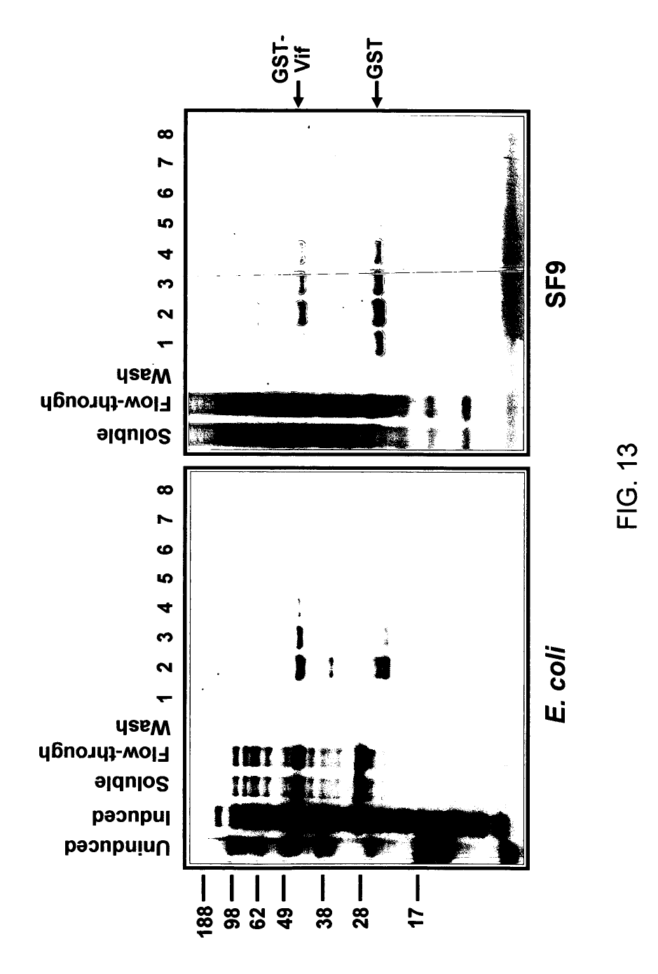

FIG. 10-13 show the production and purification of other assay components. Together, these purified assay components may be employed in the methods schematically shown in FIG. 7.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of inhibiting replication of a retrovirus in a host cell, the method comprising:
contacting a mammalian cell infected with a retrovirus with an agent that inhibits ubiquitylation activity of E1 in the infected cell, said contacting being effective to inhibit replication of the retrovirus in the cell.

2. The method of claim 1, wherein the retrovirus is human immunodeficiency virus (HIV).

3. The method of claim 2, wherein the HIV is HIV-1.

4. The method of claim 1, wherein the retrovirus delivers virion infectivity factor (Vif) into the host cell.

5. The method of claim 1, wherein the mammalian cell is a CD4⁺ cell.

6. The method of claim 1, wherein the agent inhibits E1-mediated ubiquitylation of CEM15 polypeptide in the cell.

7. The method of claim 6, wherein the agent inhibits E1-mediated activation of a ubiquitin conjugating agent that, with a ubiquitin ligating agent, facilitates ubiquitylation of CEM15 in the cell.

8. The method of claim 7, wherein the ubiquitin ligating agent is an E3.

9. The method of claim 7, wherein the ubiquitin conjugating agent is TRAC-1.

10. A method of inhibiting replication of a retrovirus in a host cell, the method comprising:
contacting a mammalian cell infected with a retrovirus with an agent that inhibits TRAC-1-mediated ubiquitylation in the infected cell, said contacting being effective to inhibit replication of the retrovirus in the cell.

11. The method of claim 10, wherein the retrovirus is human immunodeficiency virus (HIV).

12. The method of claim 11, wherein the HIV is HIV-1.

13. The method of claim 10, wherein the retrovirus delivers virion infectivity factor (Vif) into the host cell.

14. The method of claim 10, wherein the mammalian cell is a CD4⁺ cell.

15. A method of inhibiting replication of a retrovirus in a host cell, the method comprising:
contacting a mammalian cell infected with a retrovirus with an agent that promotes USP-25-mediated de-ubiquitylation of CEM15 in the infected cell, said contacting being effective to enhance USP-25-mediated de-ubiquitylation and inhibit retroviral replication in the cell.

16. The method of claim 15, wherein the retrovirus is human immunodeficiency virus (HIV).

17. The method of claim 16, wherein the HIV is HIV-1.

18. The method of claim 15, wherein the retrovirus delivers virion infectivity factor (Vif) into the host cell.

19. The method of claim 15, wherein the mammalian cell is a CD4⁺ cell.

20. A method of screening for an antiviral agent, the method comprising:
combining in a test sample
a candidate agent,
a ubiquitylation substrate polypeptide (SP),
a ubiquitin activating agent,
a ubiquitin conjugating agent,
a ubiquitin ligating agent,
a tagged ubiquitin (tag-Ub), and
a retroviral ubiquitylation-modulating protein,
said combining being under conditions suitable for ubiquitylation of the SP to produce tag-Ub-SP;
detecting a level of tag-Ub-SP;
wherein a level of tag-Ub-SP that is decreased in the presence of the candidate agent relative to a level of tag-Ub-SP in the absence of the agent indicates the agent is an antiviral agent for a retrovirus having the retro viral ubiquitylation modulator protein.

21. The method of claim 20, wherein the ubiquitylation substrate polypeptide is CEM15.

22. The method of claim 21, wherein the viral ubiquitylation modulator protein is Vif of human immunodeficiency virus (HIV).

23. The method of claim 20, wherein the ubiquitylation substrate polypeptide is CD4.

24. The method of claim 21, wherein the viral ubiquitylation modulator protein is Vpu of human immunodeficiency virus (HIV).

25. The method of claim 20, wherein the ubiquitin activating agent is E1.

26. The method of claim 20, wherein the ubiquitin conjugating agent is an E2.

27. The method of claim 20, wherein the ubiquitin ligating agent is an E3.

28. The method of claim 20, wherein the ubiquitin ligating agent is TRAC-1.

29. The method of claim 20, wherein the test sample further comprises a de-ubiquitylating agent.

30. The method of claim 20, wherein the de-ubiquitylating agent is a USP-25.

31. A method of screening for an antiviral agent, the method comprising:
combining in a test sample
a candidate agent,
a ubiquitylated complex comprising a ubiquitylation substrate polypeptide (SP) conjugated to a detectably labeled ubiquitin (tag-Ub),
a de-ubiquitylating agent, and
a retroviral ubiquitylation modulator protein,
said combining being under conditions suitable for de-ubiquitylation of the ubiquitylated complex to release tag;
detecting de-ubiquitylation of the ubiquitylated complex;
wherein an increase in de-ubiquitylation of the ubiquitylated complex in the presence of the candidate agent relative to de-ubiquitylation of the ubiquitylated complex in the absence of the agent indicates the agent is an antiviral agent for a retrovirus having the retroviral ubiquitylation modulator protein.

32. The method of claim 31, wherein the ubiquitylation substrate polypeptide is CEM15.

33. The method of claim 32, wherein the viral ubiquitylation modulator protein is Vif of human immunodeficiency virus (HIV).

34. The method of claim 31, wherein the ubiquitylation substrate polypeptide is CD4.

35. The method of claim 34, wherein the viral ubiquitylation modulator protein is Vpu of human immunodeficiency virus (HIV).

36. The method of claim 31, wherein the de-ubiquitylating agent is a USP-25 polypeptide.

37. The method of claim 31, wherein de-ubiquitylation of the ubiquitylated complex is detected by detecting tag-Ub released from the complex.

38. A method of screening for an agent that inhibits retroviral-mediated modulation of ubiquitylation in a mammalian cell, the method comprising:
contacting a candidate agent with a mammalian cell comprising a TRAC-1 polypeptide and a retroviral ubiquitylation modulator protein, said contacting being under conditions suitable for TRAC-1-mediated ubiquitylation activity; and
determining an effect of the candidate agent upon ubiquitylation activity of TRAC-1;
wherein a decrease in TRAC-1-mediated ubiquitylation in the presence of the candidate agent relative to in the absence of the agent indicates the candidate agent inhibits retroviral-mediated modulation of ubiquitylation.

39. The method of claim 38, wherein retroviral-mediated ubiquitylation is mediated by human immunodeficiency virus (HIV) virion infectivity factor (Vif).

40. The method of claim 38, wherein retroviral-mediated ubiquitylation is mediated by human immunodeficiency virus (HIV) Vpu.

41. The method of claim 38, wherein said determining is by detecting ubiquitylation of a ubiquitylation substrate protein.

42. The method of claim 41, wherein the ubiquitylation substrate protein is CEM15.

43. The method of claim 41, wherein the ubiquitylation substrate protein is CD4.

44. The method of claim 38, wherein said determining is by detecting association of TRAC-1 with a ubiquitylated E2.

45. The method of claim 44, wherein the ubiquitylated E2 is ubiquitylated with a detectably labeled ubiquitin.

46. A method of screening for an agent that inhibits retroviral-mediated modulation of ubiquitylation in a mammalian cell, the method comprising:
contacting a candidate agent with a mammalian cell comprising a de-ubiquitylation (DUB) polypeptide and a retroviral ubiquitylation modulator protein, said contacting being under conditions suitable for de-ubiquitylation activity; and
determining an effect of the candidate agent upon de-ubiquitylation activity of the DUB polypeptide;
wherein an increase in de-ubiquitylation in the presence of the candidate agent relative to in the absence of the agent indicates the candidate agent inhibits retroviral-mediated modulation of ubiquitylation.

47. The method of claim 46, wherein said determining is by detecting de-ubiquitylation of a ubiquitylated substrate protein.

48. The method of claim 47, wherein the ubiquitylatied substrate protein is CEM15.

49. The method of claim 48, wherein the retroviral ubiquitylation modulator protein is Vif of human immunodeficiency virus (HIV).

50. The method of claim 47, wherein the ubiquitylatied substrate protein is CD4.

51. The method of claim 50, wherein the retroviral ubiquitylation modulator protein is Vpu of human immunodeficiency virus (HIV).

52. The method of claim 46, wherein the DUB is USP-25.

53. The method of claim 46, wherein said determining is by detected release of a detectably labeled ubiquitin polypeptide from a cellular substrate.

54. A method of screening for ubiquitin agents that have their activity in the ubiquitylation cascade modified by a retroviral ubiquitylation modulator protein, the method comprising:
culturing a plurality of cells containing a retroviral ubiquitylation modulator protein, wherein the plurality of cells express a plurality of different ubiquitin agents, wherein the ubiquitin agent is a ubiquitin moiety, a ubiquitin activating agent, a ubiquitin conjugating agent, a ubiquitin ligating agent, or a de-ubiquitylation agent; and
screening the plurality of cells for an altered phenotype, wherein the ubiquitin agent is identified as a modulator of the phenotype.

55. The method of claim 54, wherein the retroviral ubiquitylation modulator protein is human immunodeficiency virus (HIV) virion infectivity factor (Vif).


SEQUENCE LISTING (20 sequences)

SEQ ID NO: 1 - protein, 192 aa, HIV
EEKRWIVRPTWRVPEMRIERWHSLVKYLMYRTKKLQKWFYRHHYQITWAWTCSRVIIPLGKGKLEVQGYWHLTPERGWLSTYAVGIQWYSRKYRTEVDPDTADSLIHGHYFSCFTERAIRRAIRGEKLLSCCQFPEGHKGQVGSLQYLALLAVLSNRRSRGETPTTKKLRRDNGRGLRMAKRHRRRRQQGGS

SEQ ID NO: 2 - protein, 232 aa, hiv
MSQEKHWVMRLTWKVQEEVITKWQGIVRYWMNKRNLKWEYKMHYQITWAWYTMSRYVIPLPGSGEIHVDIYWHLAPKQGWLSTYAVGIQYVSLVNDKYRTELDPNTADSMIHCHYFTCFTDRAIQQALRGNRFIFCQFPGGHKLTGQVPSLQYLALLAHQNGLRKRSQRGETRRTRNLGSQQGAVGRMAQRYGRRNQQRSQTAFWPRTPIPSMELLSGGRGETGKTHSGKGI

SEQ ID NO: 3 - protein, 214 aa, hiv
MEEEKRWIAVPTWRIPERLERWHSLIKYLKYKTKDLQKVCYVPHFKVGWAWWTCSRVIFPLQEGSHLEVQGYWHLTPERGWPSTYAVRITWYSRDLLDRCNTRLCRHFSCIALISLFTAGEVRRAIRGEQLLSCCKFPRAHRYQVPSLQYLALKVVSDVRSQGENPTWKQWRRDNRRGLRMAKQNSRGDKQRGGKPPTKGADFPGLAKVLGILA

SEQ ID NO: 4 - protein, 216 aa, hiv
MEEEKDWIVVPTWRIPGRLERWHSLIKYLKYRTGELQQVSYVPHHKVGWAWWTCSRIIFPLNKGAWLEVQGYWNLTPERGFLSSYAVRLTWYERNFYTDVTPDVADQLLHGSYFSCFSANEVRRAIRGEKILSYCNYPSAHEGQVPSLQFLALRVVQEGKNGSQGESATRKQRRRNSRRSIRLARKNNNRAQQGSGQPFAPRTYFPGLAEVLGILA

SEQ ID NO: 5 - protein, 215 aa, hiv
MEEGKNWIVVPTWRVPGRMERWHSLVKHLKYRTKDLEEVRYVPHHKVGWAWWTCSRVIFPLEGESHLEIQAYWNLTPEKGWLSSHSVRLTWYTEKFWTDVTPDCADSLIHSTYFSCFTAGEVRRAIRGEKLLSCCNYPQAHKAQVPSLQYLALVVVQQNGRPQRKGAARKQWRRDHWRGLRVARQDYRSLKQGGSEPSAPRAHFPGVAKVLGILA

SEQ ID NO: 6 - protein, 235 aa, hiv
MNPNKEWVMRVTWKVPGDLITKWQGIVRYWMRQRNLKWNYYMHYQITWAWYTMSRYVIPIGKHGEICVDLYWHLTPEQGWLSTYAVGIQYVSNLESKYRTELDPATADSIIHGHYFNCFKERAIQQALRGHRFVFCQFPEGHKSTGQVPSLQYLALLAHQNGLRERSKRGKTRRSRNLGSKQGAVGQMAKRYVTRSQPGGEAAFWERTPVPSMELLSGGRRKTWYSHDGKGLQIL

SEQ ID NO: 7 - protein, 232 aa, hiv
MNQEKEWVMRVTWKVPEELITKWQGIVRYWMRTRKLDWKYRMHYQITWAWYTMSRYEIPLGQHGSIHVDLYWHLTPEKGWLSTYAEGIQYLSNRDPWYRTELDPATADSLIHTHYFTCFTERAIRKALLGQRFTFCQFPEGHKKTGQVPSLQYLALLAHQNGLRQRSQRSKTGGTRNMGFEQGAVGRMAKRHARRYQSGSQDAFWARAPVPSMELLSGGGRKESHSHARKGL

SEQ ID NO: 8 - protein, 219 aa, hiv
MEREKQWIVRVVWRVSERQISRWRGIVTYKIRNKQLPWEYRHHWQVQWQFWTYSQFIIPLSKDDYIEVNIYHNLTPERGWLSSHGVGLSYYHQKGYKTEVDPGTADRMIHLYYFNCFTDRAIQQAIRGEKYTWCTFKEGHKGQVQSLQLLALVAYTNGIRKRSKRTFTRMAGNLGSRQGAMGRMATRHAQGSKRRSQKALWNEHANPSMELLCRGGKET

SEQ ID NO: 9 - protein, 193 aa, hiv
MENRWQVMIVWQVDRMRIKTWNSLVKYHIYRSKKARGWFYRHHYDHPNPKVASEIHIPFRDYSKLIVTTYWALSPGERAWHLGHGVSIQWRLGSYVTQVDPFTADRLIHSQYFDCFAETAIRRAILGQLVAPRCEYKEGHRQVGSLQFLALKALISERRHRPPLPSVAKLTEDRWNKHQRTKVHQENLTRNGH

SEQ ID NO: 10 - protein, 192 aa, hiv
MENRWQVMIVWQVDRMRIRTWKSLVKHHMYISKKAKGWFYRHHYESTHPRVSSEVHIPLGDAKLVITTYWGLHTGEREWHLGQGVAIEWRKKKYSTQVDPGLADQLIHLHYFDCFSESAIKNAILGYRVSPRCEYQAGHNKVGSLQYLALAALITPKKTKPPLPSVKKLTEDRWNKPQKTKGHRGSHTMNGH

SEQ ID NO: 11 - protein, 80 aa, hiv
QPLLILAIVALVVALIIAIVVWTIVYIEYRKLKRQRKIDRLIDRIRERAEDSGNESEGDTEELSTLVEMGNHDLGDANNL

SEQ ID NO: 12 - protein, 88 aa, hiv
MTLLVGLVLILVGLIAWNICIWGYIIKWGYRRYKRHRLETEIERLNLILRERAEDSGNESNGEEEERLEQLIHNYNHNNHFANPMFDL

SEQ ID NO: 13 - protein, 85 aa, hiv
MQHKDLLIVIISSALLFINVILWTYNLKTYLEQRKQDRREREILERLERIRKIRDDSDYESNGEEEQEVMDLVHSYGFANPMFEL

SEQ ID NO: 14 - protein, 81 aa, hiv
MSNLLAIGIAALIVALIITIVVWTIAYIEYKKLVRQRKINRLYKRISERAEDSGNESEGDAEELAALGEVGPFIPGDINNL

SEQ ID NO: 15 - protein, 81 aa, hiv
MLPLATLSIVGLIVALILAIVVWTIVFIEYKKIKKQKKIDWLIKRISERAEDSGNESEGDTEELATMVDMGHLRLLDVNDL

SEQ ID NO: 16 - protein, 81 aa, hiv
MTSLEIYAIVALIVALIIVIVVWTLAGIEYKKLLKQRKIDRLIKKIRERAEDSGNESDGDIDELSKLVGVGNYDLGDVNNL

SEQ ID NO: 17 - protein, 80 aa, hiv
MYILGLGIGALVVTFIIAVIVWTIVYIEYKKLVRQKKIDRLIERIGERAEDSGNESDGDTEELSKLMEMGHLNLGYVADL

SEQ ID NO: 18 - protein, 81 aa, hiv
MQSLEISAIVGLIVAFIAAIIVWTIVLVQYREIRKQRKVERLIDRIRERAEDSGNESEGDREKLTTLMKMGDFDPWVGDNL

SEQ ID NO: 19 - protein, 81 aa, hiv
MQALEIAAIVGLVVAFLAAIVVWTIVFIQYREIRKQKKIEKLLDRIRERAEDSGNESEGDTDELATLMEMGDFDPWVGDNL

SEQ ID NO: 20 - protein, 81 aa, hiv
MQSLVILAIVAVVAALIIAIVVWTIVFIECRRLSRQRQIDWLIDRIRERAEDSGNESEGDKEELSALVEMGHHAPWNIDDM